(19) United States
(12) Patent Application Publication
(10) Pub. No.: US 2004/0053388 A1
(43) Pub. Date: Mar. 18, 2004

(54) Detection of protein conformation using a split ubiquitin reporter system

(76) Inventors: Jorg H. Eckert, Witten (DE); Nils Johnsson, Eggenstein-Leopoldshafen (DE); Xavier Raquet, Xhendelesse (BE)

Correspondence Address: Ropes & Gray, One International Place, Boston, MA, 02110-2624, US

(21) Appl. No.: 10/250,614

(22) PCT Filed: Jan. 3, 2002
(86) PCT No.: PCT/US02/00325

(51) Int. Cl. C12P 21/04; C12N 9/12; C07K 14/72; C07K 14/47

(52) U.S. Cl. 435/194; 530/350; 530/399; 435/69.7; 435/320.1; 435/325

(57) ABSTRACT

The invention provides methods and reagents for monitoring protein structure by an intrapolypeptide split-ubiquitin assay.

### REFERENCE TO RELATED APPLICATIONS

This application claims priority to U.S. Provisional Application 60/259827, filed on Jan. 4, 2001, the specifications of which is incorporated by reference herein.

### BACKGROUND OF THE INVENTION

Protein interactions facilitate most biological processes including signal transduction and homeostasis. The elucidation of particular interacting protein partners facilitating these biological processes has been advanced by the development of in vivo “two-hybrid” or “interaction trap” methods for detecting and selecting interacting protein partners (see Fields & Song, Nature 340: 245-6, 1989; Gyuris et al., Cell 75: 791-803, 1993). These methods rely upon the reconstitution of a nuclear transcriptional activator via the interaction of two binding partner polypeptides—i.e. a first polypeptide fused to a DNA binding domain and a second polypeptide fused to a transcriptional activation domain. When the first and the second polypeptides interact, the interaction can be detected by the activation of a reporter gene containing binding sites for the DNA binding domain. For this method to work, both proteins need to be localized to the nucleus. Accordingly, the interaction of polypeptides which are normally localized to other compartments may not be detected because of the absence of other non-nuclear polypeptide components which facilitate the interaction or particular non-nuclear post-translational modifications which fail to occur in the nucleus or because the interacting proteins fail to fold properly when localized to the nuclear compartment. In particular, the nuclear two-hybrid assay is ill suited to the detection of protein interactions occurring within or at the surface of cellular membranes.

The Split Ubiquitin Protein Sensor is described in U.S. Pat. Nos. 5,585,245 and 5,503,977. In brief, the “split ubiquitin” method is a means of detecting protein protein interactions that relies in part upon the fact that isolated amino- and carboxyl fragments of ubiquitin (e.g. comprising amino acids 1 to 37 and 38 to 76 respectively) are able to spontaneously associate to reconstitute a bimolecular ubiquitin polypeptide complex that is recognized by ubiquitin specific proteases (UBPs). These proteases can then actively cleave the polypeptide bond between amino acid residue 76 of the carboxyl fragment of ubiquitin and any linked polypeptide. If this linked polypeptide is a reporter which can be detected from the carboxyl-terminal ubiquitin protein fragment, then the association of amino and carboxyl ubiquitin fragments can be monitored by the release of the reporter activity. This “reassociation” of ubiquitin amino and carboxyl fragments can be made dependent upon the association of two heterologous polypeptides by mutating one or both of the ubiquitin fragments (e.g. by a conservative amino acid substitution of a neutral amino acid residue) so that they fail to “reassociate” without the aid of linked heterologous binding partners. The two heterologous polypeptides (i.e. a first polypeptide and a second polypeptide) are provided as fusions to the mutant amino and/or carboxyl ubiquitin fragments. In addition, the carboxyl ubiquitin fragment is fused at its C-terminus to a reporter gene. The resulting two fusions have the structures 1^{st} polypeptide-N-Ub*₍₁₋₃₇₎ and 2^{nd} polypeptide-C-Ub₍₃₈₋₇₆₎-reporter. In the absence of the interaction of the first and second polypeptides, the altered ubiquitin amino and carboxyl fragments fail to associate. In contrast, association of the first and second polypeptides results in reassembly of the amino Ub* and carboxyl Ub fragments and cleavage of the carboxyl Ub-reporter bond, thereby releasing free reporter. If the reporter is active upon its release, but inactive while fused to the carboxyl fragment of ubiquitin, its activity can be monitored in a screen for polypeptide binding partners (see U.S. Pat. Nos. 5,585,245 and 5,503,977). The split-Ub assay has been shown to detect stable interactions between soluble proteins, between membrane proteins, and a transient interaction between substrate and transporter during protein translocation in vivo (Dünnwald et al., Mol. Biol. Cell. 10: 329-44, 1999; Stagljar et al. Proc. Natl. Acad. Sci USA 95:5187-92, 1998; Wellhausen et al., FEBS Lett. 453: 299-304, 1999; Wittke et al., Mol. Biol. Cell. 10: 2519-30, 1999).

The split ubiquitin method has been applied to measurements of protein/protein interpolypeptide interactions, however not to measuring intramolecular or intrapolypeptide interactions, such as occur during polypeptide folding. The proper folding of a protein to its mature conformation is a particularly important step in the expression of any biologically active protein. For example, proper folding is essential to the activity of proteins encoding enzymatic activities as well as to those serving structural roles in the cell. Indeed two interacting proteins, must each first fold appropriately so that a proper conformation for each to interact is first adopted. Protein folding and polypeptide conformation have many important biological consequences.

Mutations that cause an abnormal phenotype in genetically accessible organisms or a disease in humans often alter the conformation or metabolic stability of the corresponding protein (Booth et al., Nature 385: 787-93, 1997; Radford et al., Cell 97: 291-98, 1999; Wong et al., Proc. Natl. Acad. Sci. USA 96: 8438-42, 1999). For example, Alzheimer's disease is thought to be caused by an alternatively-folded conformation of the beta-amyloid protein (see e.g. Roher et al., Biochim Biophys Acta 1502: 31-43, 2000), which causes an oligomerization of the protein that is initiated intracellularly (Walsh et al., Biochemistry 39: 10831-9, 2000). Accordingly, the ability to monitor protein conformation in vivo would be useful in assays for therapeutic agents which prevent inappropriate amyloid protein folding and/or oligomerization. An increasing number of proteins are known to adopt their active conformation only upon binding to a ligand, a cofactor or a binding partner (Daughdrill et al., Nat. Struct. Biol. 4: 285-91, 1997; Kriwacki et al., Proc. Natl. Acad. Sci. USA 93: 11504-09, 1996; Mateu et al., Nat. Sruct. Biol. 6: 191-98, 1999; Shakhnovich, Nat. Struct. Biol. 6: 99-102, 1999; Zurdo et al., Biochemistry 36: 9625-35, 1997). To follow these transitions or to understand the effect of a particular mutation therefore requires the ability to monitor the conformation of the affected protein. Since it is now generally accepted that the cellular environment can influence the efficiency of folding and the conformational stability of a protein, it should be of great interest to have a method that allows the detection and measurement of these conformational alterations (Ellis et al., Curr. Opin. Struct. Biol. 9: 102-10, 1999).

Conformational transitions are usually accompanied by changes in chemical and physical parameters of the protein. Existing biophysical techniques, such as circular dichroism or x-ray crystallograph, that measure these parameters generally relay on purified samples and cannot monitor conformation alterations in living cells. To be able to study these processes in live cells, one alternative to the established methods is to attach conformation-specific probes to the protein of interest. The instant invention provides methods and reagents for measuring polypeptide conformation using a unique intrapolypeptide split-ubiquitin method.

### SUMMARY OF THE INVENTION

The invention provides certain methods and reagents useful for detecting and measuring polypeptide conformational changes. In preferred embodiments the method of the invention allows for the detection of a conformational change in a polypeptide resulting from a mutational alteration in the polypeptide sequence or from contact of the polypeptide with a test compound. In certain preferred embodiments the method of the invention utilizes a fusion protein having the general structure N_{ub}—X—C_{ub}, where N_{ub} is an amino-terminal ubiquitin domain or mutant amino-terminal ubiquitin domain, C_{ub} is a carboxy-terminal ubiquitin domain and X is a polypeptide of interest—preferably a nonubiquitin polypeptide. In certain preferred embodiments, the fusion further comprises a reporter polypeptide fused to the carboxy-terminus of the C_{ub} domain and the fusion protein comprises a fusion protein reporter moiety having the general structure: N_{ub}—X—C_{ub}-Reporter. In preferred embodiments, the reporter is URA3, thymidine kinase or Green Fluorescent Protein (GFP). In particularly preferred embodiments the N_{ub} domain is a wild-type or mutant amino-terminal ubiquitin domain having mutated amino acid replacements at positions three and thirteen of ubiquitin such as: Nᵥᵢ, Nᵢₐ, Nᵥₐ, N_{ig}, N_{vg}, Nₐᵢ, Nₐₐ, N_{ag}, N_{gi}, N_{ga}, or N_{gg} respectively. In particularly preferred embodiments, the protein of interest is Guk1, Fpr1, Sec62p, beta-amyloid, G-proteins or p53.

In a preferred method of the invention, a polypeptide conformational alteration resulting from a mutational alteration the polypeptide sequence or from contact of the polypeptide with a test compound is detected by first measuring the reporter activity from the N_{ub}—X—C_{ub}-reporter and then comparing it to the reporter activity from the N_{ub}—X—C_{ub}-reporter following mutational alteration of the polypeptide of interest or contact of the polypeptide of interest with a test compound. A changes in the level of the fusion protein reporter activity following mutation or contact with the test compound indicate that the mutational alteration or test compound causes a conformational change in the protein. Preferably, the method of the invention is performed in vivo, with the polypeptide reporter fusion expressed in an intact cell in situ or in a cultured cell existing in vitro. In particularly preferred embodiments, the test compound is a polypeptide or a small molecule and is supplied from a library of test polypeptides or library of test small molecules.

One aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide selected from the group consisting of: GuK1, Fpr1, Sec62p, beta-amyloid, p53, calmodulin, estrogen receptor alpha (ERα), FKBP, G-protein, VHL, tyrosine kinases, Src, Abl, Epidermal Growth Factor receptor (EGFR), Protein Kinase A (PKA), Protein Kinase C (PKC), Cyclophillins, Cyclin Dependent Kinases (CDKs), Cyclins, a protein of therapeutic, physiological or biological interest or variants/fragments thereof.

A related aspect of the invention provides a fusion protein comprising the structure N_{ub}—X—C_{ub}—RM, wherein C_{ub} is a carboxy-terminal ubiquitin domain, RM is a reporter moiety fused to the carboxy-terminus of the C_{ub} domain, X is a nonubiquitin polypeptide, and N_{ub} is a mutant amino-terminal ubiquitin domain selected from the group consisting of: N_{,vi}, Nᵥₐ, N_{vg}, Nₐᵢ, Nₐₐ, N_{ag}, N_{gi}, N_{ga}, and N_{gg}.

A related aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide, wherein RM is a selectable marker.

A related aspect of the invention provides a fusion protein comprising the structure N_{ub}X—CC_{ub}RM, wherein C_{ub} is a carboxy-terminal ubiquitin domain, RM is a reporter moiety fused to the carboxy-terminus of the C_{ub} domain, X is a nonubiquitin polypeptide, and N_{ub} is a mutant amino-terminal ubiquitin domain which has altered affinity for C_{ub} chosen such that for a given X polypeptide it just inhibits or just allows the reconstitution of a quasi-native ubiquitin and hence cleavage of RM from the fusion protein.

A related aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a non-yeast nonubiquitin polypeptide.

A related aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is a reporter moiety fused to the carboxy-terminus of the C_{ub} domain, wherein upon cleavage of the C_{ub}—RM junction, the first amino acid of the released RM is an amino acid other than methionine.

In one embodiment, the first amino acid of the cleaved RM is Arginine, Lysine, Histidine, Phenylalanine, Tryptophan, Tyrosine, Leucine, Aspartate, Glutamate, Cysteine, Asparagine, Glutamine or Isoleucine.

Another aspect of the invention provides a polynucleotide sequence encoding any one of the fusion proteins of the instant invention.

Another aspect of the invention provides a host cell harboring a polynucleotide sequence encoding any one of the fusion proteins of the instant invention.

Another aspect of the invention provides a method of detecting a conformational change in a polypeptide resulting from a mutational alteration in the polypeptide sequence comprising: (a) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety, wherein upon cleavage of the C_{ub}—RM junction, the first amino acid of the released RM is an amino acid other than methionine; and, (b) measuring a second fusion protein reporter moiety activity from a N_{ub}—X′—C_{ub}—RM, wherein X′ is a mutationally altered form of polypeptide X; wherein a change in the level of the second fusion protein RM activity relative to the first fusion protein RM activity indicates that the polypeptide has undergone a conformation change resulting from the mutational alteration.

A related aspect of the invention provides a method of detecting a conformational change in a polypeptide resulting from a point mutation or an insertion/deletion of no more than 3 amino acids in the polypeptide sequence comprising: (a) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety; and, (b) measuring a second fusion protein reporter moiety activity from a N_{ub}—X′—C_{ub}—RM, wherein X′ is a point mutation or a deletion/insertion of no more than three amino acids form of polypeptide X; wherein a change in the level of the second fusion protein RM activity relative to the first fusion protein RM activity indicates that the polypeptide has undergone a conformation change resulting from the mutational alteration.

A related aspect of the invention provides a method of detecting a conformational change in a polypeptide resulting from a stimulus comprising: (a) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety; and, (b) measuring a second fusion protein reporter moiety activity from a N_{ub}—X′—C_{ub}—RM, wherein X′ is the X polypeptide which has been altered by the stimulus; wherein a change in the level of the second fusion protein RM activity relative to the first fusion protein RM activity indicates that the polypeptide has undergone a conformation change resulting from the stimulus.

In a preferred embodiment, the stimulus is an alteration in environmental factor, which can be pH change, temperature change, pressure change, redox-state change or ionic strength change.

In another preferred embodiment, the stimulus is a post-translational modification of the X protein, which can be phosphorylation, methylation, prenylation, acetylation, palmitoylation, myristoylation, reduction, oxidation, glycosylation, proteolytic cleavage, sulfation, hydroxylation, carboxylation, or the covalent linkage of ubiquitin-like proteins (Ubl) to X such as ubiquitination or sumoylation.

In another preferred embodiment, the stimulus is contacting the X protein with a test compound in trans, which test compound is selected from the group consisting of: a polypeptide, a hormone, a steroid, an ion, a polynucleotide, an oligosaccharide, a lipid, an enzyme substrate, a gas molecule, a small molecule, a co-factor, a vitamin, a metal ion, and a nucleotide phosphate.

In another preferred embodiment, the nonubiquitin polypeptide of interest is selected from the group consisting of: Guk1, Fpr1, Sec62p, beta-amyloid, p53, calmodulin, estrogen receptor alpha (ERα), FKBP, and G-protein, VHL, tyrosine kinases, Src, Abl, Epidermal Growth Factor (EGF) receptor, Protein Kinase A (PKA) Protein Kinase C (PKC), Cyclophillins, Cyclin Dependent Kinases (Cdk), Cyclins, a protein of therapeutic, physiological or biological interest or variants/fragments thereof.

In another preferred embodiment, the N_{ub} domain is an amino-terminal ubiquitin domain or a mutant amino-terminal ubiquitin domain selected from the group consisting of: Nᵢₐ, N_{ig}, Nᵥᵢ, Nᵥₐ, N_{vg}, Nₐᵢ, Nₐₐ, N_{ag}, N_{gi}, N_{ga}, and N_{gg}.

In another preferred embodiment, the reporter moiety (RM) is a selectable marker.

In another preferred embodiment, the first amino acid of the RM is a non-methionine residue when the RM is released by cleavage of the C_{ub}—RM junction by a ubiquitin-specific protease (UBP).

In another preferred embodiment, N_{ub} is a mutant amino-terminal ubiquitin domain which has altered affinity for C_{ub} chosen such that for a given X polypeptide it just inhibits or just allows the reconstitution of a quasi-native ubiquitin and hence cleavage of RM from the fusion protein.

In other preferred embodiment, X is a non-yeast nonubiquitin polypeptide.

In another preferred embodiment, at least one step is performed in a host cell expressing a ubiquitin-specific protease.

Another aspect of the invention provides a method to identify a compound which can change the conformation of a protein upon contacting the protein, comprising: (a) providing a plurality of test compounds which are not known to be able to cause the conformation change of the protein; (b) testing each compound by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin protein of interest, and RM is a reporter moiety; and, measuring a second fusion protein reporter moiety activity from a N_{ub}—X′—C_{ub}—RM, wherein X′ is the X protein which has been altered by the test compound; wherein a change in the level of the second fusion protein RM activity relative to the first fusion protein RM activity indicates that the X protein has undergone a conformation change resulting from contacting the test compound, thereby identifying a compound which can change the conformation of the protein.

In a preferred embodiment, the method further comprises formulating the identified compound into a pharmaceutical composition.

In another preferred embodiment, the plurality of test compounds is a library of compounds which comprises 2 to 10 test compounds, or greater than 10 test compounds. Preferably 10 to 500, 500 to 10,000 or greater than 10,000 test compounds.

Another aspect of the invention provides a method to identify a mutation in a protein which leads to the conformation change of the protein, comprising: (a) generating a plurality of candidate mutations of the protein; (b) testing each candidate mutation by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin protein of interest and RM is a reporter moiety; and, measuring a second fusion protein reporter moiety activity from a N_{ub}—X′—C_{ub}—RM, wherein X′ is a mutation altered form of the X protein harboring the candidate mutation; wherein a change in the level of the second fusion protein RM activity relative to the first fusion protein RM activity indicates that the X protein has undergone a conformation change resulting from the candidate mutation, thereby identifying a mutation which can change the conformation of the protein.

Another aspect of the invention provides a method to identify a protein which changes conformation upon contacting a given compound or encountering an alteration in environmental factor, comprising: (a) providing a plurality of test proteins; (b) testing each protein X by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is the nonubiquitin test protein, and RM is a reporter moiety; and, measuring a second fusion protein reporter moiety activity from a N_{ub}—X′—CC_{ub}—RM, wherein X′ is the X protein which has been altered by contacting the given compound or by the given alteration in environmental factor; wherein a change in the level of the second fusion protein RM activity relative to the first fusion protein RM activity indicates that the X protein has undergone a conformation change resulting from the given alteration in environmental factor or contacting the given compound, thereby identifying a protein which changes conformation upon contacting a given compound or encountering an alteration in environmental factors.

Another aspect of the invention provides a method to conduct a business, comprising: (a) by a suitable method of the invention, identifying one or more compounds which change the conformation of a polypeptide; (b) conducting therapeutic profiling of said identified compounds, or other derivatives thereof, for using the compounds in therapy for a condition; and, (c) formulating a pharmaceutical preparation including one or more compounds identified in (b) as a product having an acceptable therapeutic profile.

In a preferred embodiment, the business method further comprises establishing a distribution system for distributing said product for sale.

In another preferred embodiment, the business method further includes establishing a sales group for marketing the product

Another aspect of the invention provides a method to conduct a business, comprising: (a) by a suitable method of the invention, identifying one or more compounds which change the conformation of a polypeptide; (b) conducting therapeutic profiling of said identified compounds, or other derivatives thereof, for using the compounds in therapy for a condition; and, (e) licensing to a third party, the rights for further development of compounds and/or formulating a pharmaceutical preparation including one or more compounds identified in (b) to affect conformation change of the polypeptide for treatment of the condition.

Another aspect of the invention provides a method to conduct a business, comprising: (a) by one or more suitable methods of the invention, generating information or data, or identifying compounds, proteins or mutations/variants/derivatives thereof, (b) licensing, selling, providing for consideration or access to said information, said data, said identified compounds, proteins or mutations/variants/derivatives thereof.

Another aspect of the invention provides a kit for detecting or identifying alterations in the conformation of an X protein, comprising a panel of at least two vector constructs for expressing fusion proteins of the general structure N_{ub}—X—C_{ub}—RM, wherein each vector construct comprises a coding sequence for N_{ub}, an amino-terminal ubiquitin domain or a mutant amino-terminal ubiquitin domain selected from Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, or N_{gg}; a coding sequence for C_{ub}, a carboxy-terminal ubiquitin domain; a coding sequences for RM, a reporter moiety fused to the carboxy-terminus of the C_{ub} domain; and at least one cloning site or multicloning site for subcloning the X-protein in-frame with both the N-terminal N_{ub} and the C-terminal C_{ub}—RM moieties; and wherein at least one vector construct expresses a mutant N_{ub} fusion protein.

In one embodiment, the kit further comprises a host cell for expressing said fusion proteins from said vector constructs.

In one embodiment, the kit farther comprises instructions for detecting or identifying alterations in protein conformation by using the vector constructs.

Another aspect of the invention provides a method for detecting a conformation change of a protein resulting from a stimulus, comprising: (a) measuring a first spectrum of fusion protein reporter moiety activity from a first panel of at least two fusion proteins, each different from the other, comprising the general structure N_{ub}—X—C_{ub}—RM, wherein N_{ub} is an amino-terminal ubiquitin domain or mutant amino-terminal ubiquitin domain selected from at least one of Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, and N_{gg}, C_{ub} is a carboxy-terminal ubiquitin domain, X is the protein, and RM is a reporter moiety; (b) measuring a second spectrum of fusion protein reporter moiety activity from a second panel of fusion proteins comprising the general structure N_{ub}—X′—C_{ub}—RM, wherein the second panel of N_{ub} and C_{ub} fusion proteins are the same as the first panel of N_{ub} and C_{ub} fusion proteins, X′ is the X protein resulting from treating the X protein with the stimulus, and RM is a reporter moiety; (c) comparing the first and second spectra of fusion protein reporter moiety activity; wherein a shift in the spectrum of reporter moiety activity indicates that the protein has undergone a conformation change resulting from the stimulus.

In a preferred embodiment, the stimulus is a mutational alteration of the X protein, an alteration in environmental factor, a post-translational modification of the X protein, or contacting the X protein with a test compound in trans.

Another aspect of the invention provides a composition comprising: (a) a fusion protein comprising the structure N_{ub}—X—C_{ub}—RM, wherein N_{ub} is an amino-terminal ubiquitin domain or a mutant an amino-terminal ubiquitin domain, C_{ub} is a carboxy-terminal ubiquitin domain, RM is a reporter moiety fused to the carboxy-terminus of the C_{ub} domain and X is a nonubiquitin polypeptide; and, (b) a compound that when brought in to contact with, causes conformational change in polypeptide X; and/or, (c) a fusion protein comprising the structure N_{ub}—X′—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain and X′ is the X nonubiquitin polypeptide which has been altered by a stimulus.

Another aspect of the invention provides a method of controlling activity of a target gene, comprising: (a) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is a reporter moiety fused to the carboxy-terminus of the C_{ub} domain, wherein the reporter moiety is a gene activating moiety; (b) treating the X polypeptide with a stimulus, thereby causing the cleavage of the RM as a result of a conformational change of the X polypeptide; wherein the released RM controls activity of the target gene.

Another aspect of the invention provides a method of controlling activity of a protein, comprising: (a) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is the protein fused to the carboxy-terminus of the C_{ub} domain, wherein upon cleavage of the C_{ub}—RM junction, the first amino acid of the released RM is an amino acid other than methionine; (b) treating the X polypeptide with a stimulus, thereby causing the cleavage of the RM as a result of a conformational change of the X polypeptide; wherein the released RM is degraded by N-end rule components, thereby controlling activity of the protein.

In a preferred embodiment, the stimulus is an alteration in environmental factor, a post-translational modification of the X protein, or contacting the X protein with a test compound in trans.

Another aspect of the invention provides a kit for measuring or detecting protein conformation change caused by a stimulus, comprising: (a) one or more vector constructs for expressing fusion proteins of the general stricture N_{ub}—X—C_{ub}—RM, wherein each vector construct comprises a coding sequence for N_{ub}, an amino-terminal ubiquitin domain or a mutant amino-terminal ubiquitin domain selected from Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, or N_{gg}; a coding sequence for C_{ub}, a carboxy-terminal ubiquitin domain; a coding sequences for RM, a reporter moiety fused to the carboxy-terminus of the C_{ub} domain; and at least one cloning site or multicloning site for subcloning the X-protein in-frame with both the N-terminal N_{ub} and the C-terminal C_{ub}—RM moieties; and wherein at least one vector construct expresses a mutant N_{ub} fusion protein; (b) an instruction for using the vector construct of (a) to measure/detect protein conformation change caused by the stimulus.

In a preferred embodiment, the instruction is not physically associated with the vector constructs of (a). For example, the instruction can be posted on a website, or updated periodically, or accessible as a published document.

In another preferred embodiment, the stimulus is an alteration in environmental factor, a post-translational modification of the X protein, or contacting the X protein with a test compound in trans.

Another aspect of the invention provides a method to detect or measure an alteration of an environmental factor or the presence of a compound in a sample comprising the steps: (a) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide which changes confirmation from said alteration in environmental factor or presence of said compound; (b) contacting the fusion protein with the environment or the sample containing the compound; and, (c) measuring the degree of cleavage of the reporter moiety (RM) from the fusion protein; wherein a change in the degree of RM activity compared to a standard or control indicates an alternation in said environmental factor or the presence of said compound in the sample.

### DESCRIPTION OF THE FIGURES

FIG. 1 Application of Split-Ub to detect conformational alterations in proteins. (a) A protein (green circle) whose structure brings N- and C-terminus into close proximity stimulates the reassembly of the coupled N_{ub} and C_{ub} (yellow) The reassembled Ub is recognized by the UBPs and the Dha reporter (red) is cleaved off from the C-terminus of C_{ub}. (b) A protein (blue circle) whose structure spatially separates the N- from the C-terminus interferes with the reassociation of the coupled N_{ub} and C_{ub}. The C_{ub}-moiety alone is not recognized by the UBPs and no release of the Dha occurs. (c) The unfolded chain (orange line) of the protein in either (a) or (b) neither interferes nor stimulates the reassociation of the coupled Ub peptides. Cleaved and uncleaved fusion protein coexist.

FIG. 2 Characterization of new N_{ub}-mutants and cleavage pattern of N_{ub}-Guk1-F—C_{ub}-Dha. (a) Cells expressing Ub-Dha which contained the indicated residues at positions three and thirteen of N_{ub} were incubated in medium containing 100 μM CuSO₄ to induce the expression of the constructs. Proteins were extracted after one hour of induction, separated by a 12.5% SDS-PAGE and probed with anti-HA antibody after transfer onto nitrocellulose. Uncleaved Ub-Dha is seen for the Ub_{ga}-, and the Ub_{gg}-, but not for the Ub_{gi}-mutant. (b) As in (a) but cells were expressing Guk1p extended with the FLAG epitope at its C-terminus and sandwiched between the different N_{ub} and C_{ub}-Dha. Extracts were loaded onto the gel in an order according to the expected increase in the fraction of cleaved fusion protein except for the N_{gi}-construct to allow for the alignment with the blot in (a). (c) Quantitative chemiluminescence analysis of the experiment shown in (b). Bars represent the percentage of cleaved Dha compared to the sum of cleaved and uncleaved fusion protein. Note that in contrast to (b), the order of the N_{ub} strictly accords to the decreasing efficiency of the N_{ub}—C_{ub} reassociation.

FIG. 3 Intrapolypeptide split-ubiquitin assay applied to visualizing different spatial arrangements of N- and C-termini. (a) Cells expressing N_{ub}-Guk1-C_{ub}-Dha containing a subset of the different N_{ub} mutants were extracted after one hour of induction by CuSO₄ and proteins detected with anti-HA antibody after 12.5% SDS-PAGE separation and transfer onto nitrocellulose. The analysis of the constructs carrying the N_{ub} with a higher affinity for C_{ub} than N_{vg} and thus inducing complete cleavage is not shown. (b) Same analysis as in (a) except for cells expressing N_{ub}-Fpr1-C_{ub}-Dha constructs: (c) Same analysis as in (a) except for cells expressing N_{ub}—C_{ub}Dha constructs containing a mutated version of Fpr1p (Fpr1MC). (d) Quantitative chemiluminescence analysis of the experiments shown in (a)-(c). White bars indicate the GUK1-, gray bars the FPR1-, and black bars the FPR1MC-constructs. Cartoons illustrating the arrangements of the N and C-termini in Guk1p and Fpr1p are depicted left of the immunoblots in (a) and (b). The wavy line next to the immunoblot in (c) indicates the changing distance between the N- and the C-terminus in an unfolded structure.

FIG. 4 Intra-versus inter-molecular reassociation of the coupled N_{ub} and C_{ub}. (a) Cells coexpressing Fpr1-C_{ub}-Dha with Nᵢᵢ-, Nᵢₐ-, N_{ig}-Fpr1-ha or an empty plasmid or coexpressing Guk1-C_{ub}-Dha with Nᵢᵢ-, Nᵢₐ-, N_{ig}-Guk1-ha or an empty plasmid were extracted and processed for immunoblotting with anti-ha antibody, (b) Cells expressing N_{vg}-Fpr1-C_{ub}-Dha were labeled with ³⁵S-methionine for 5 minutes and then chased with cold methionine in the presence of cycloheximide. Cells were extracted after 0, 30 and 60 minutes and subjected to immunoprecipitation with anti-ha antibody.

FIG. 5 Application of the intrapolypeptide split-ubiquitin method to monitoring destablizing mutations in three proteins with different N- and C-terminal topologies. (a) Fpr1p carries the N- and C-terminus on opposite faces of the molecule. An immunoblot of the extracts of cells expressing the N_{vg}-Fpr1p-C_{ub}-Dha and the corresponding constructs of the different mutants of Fpr1p is shown together with the quantification of four independent experiments by chemiluminescence. (b) Guk1p carries the N- and C-terminus in spatial proximity. The analysis of N_{vg}-Guk1-C_{ub}-Dha and mutants thereof was performed as in (a). (c) Sumo1p contains a flexible linker connecting the N-terminus to the compact domain of the protein. The analysis of N_{vg}-Sumo1-C_{ub}-Dha and mutants thereof was performed as in (b). In (b) and (c), only the quantification of the depicted experiments are shown.

FIG. 6 Demonstration that an easily observable phenotype reveals mutations which influence the stability or the conformation of proteins. (a) (1) Proteins (green circle) that stimulate the reassociation of the coupled N_{ub} and C_{ub} induce the efficient release of the reporter RUra3p (red) by the UBPs. The exposed N-terminal arginine of the RUra3p channels the reporter into the N-end rule pathway of protein degradation. The cells are uracil auxotroph. (2) Proteins (blue circle) interfering with the reassembly of the coupled Ub-peptides keep the active RUra3p reporter C-terminally attached. The cells are uracil prototroph. (b) 10⁵, 10⁴, 10³, 10² and 10¹ yeast cells expressing the different N_{ub}—X—C_{ub}—RUra3p fusion proteins as indicated were spotted onto medium lacking uracil and tryptophan to select for the presence of the plasmid. Cells were incubated for three days at 30° C.

FIG. 7 A single mutation causes the unfolding of the N-terminal domain of Sec62p. (a) Cells expressing the N-terminal domain of the wild type Sec62p, of sec62-1p and of sec62-141p were extracted after one hour of induction with CuSO₄ and the extracts were analyzed by immunoblotting with anti-HA antibody recognizing the Dha module of the cleaved and uncleaved fusion protein. The amino acid exchanges in the mutants are indicated on top of the gel. (b) Intermolecular reassociation of N_{ub} and C_{ub}, each coupled to a different ΔC125 Dha molecule. Cells containing ΔC125-C_{ub}-Dha or an empty plasmid and coexpressing different N_{ub}-ΔC125-Dha were extracted after one hour of induction with CuSO₄ and extracts probed after 12.5% SDS-PAGE with anti-HA antibody.

FIG. 8 The three potential outcomes of the split-Ub experiment. A) Ste18p is bound to Ste4p. The structures of Ste18p keeps N_{ub} and C_{ub} at a distance that inhibits their efficient reassociation. The RUra3p reporter is not cleaved off and the cells grow on SD-ura. B) Ste18p maintains its conformation in the absence of Ste4p. The cells can grow on SD-ura. C) Ste18p unfolds or adopts a very different conformation in the absence of Ste4p. The coupled N_{ub} and C_{ub} can reassociate, the RUra3p reporter is cleaved off and degraded. The cells cannot grow on SD-ura.

FIG. 9 A) Ste18₉₁p, an N-terminally truncated form of Ste18p that was used for the experiments, is functional. The halo of non-growing cells around a filter disk soaked with α-factor documents the functionality of the protein. The cells containing an empty vector instead are unaffected by the mating hormone. B) Cells lacking a functional N end rule and expressing Nᵢₐ-Ste18₉₁-C_{ub}—RUra3 or expressing N_{vg}-Ste18₉₁-C_{ub}—RUra3p grow on plates lacking uracil. In contrast wild-type cells that express Nᵢₐ-Ste18₁₉-C_{ub}—RUra3 do not grow on SD-ura-trp whereas the cells expressing N_{vg}-Ste18₉₁-C_{ub}—RUra3p still grow. Two different transformants are shown. Cells were grown for 2.5 days at 30° C. (C) Cells that contain N_{vg}-Ste18₉₁-C_{ub}—ruRA3 together with an empty vector (lane 1) or Nᵢₐ-Ste18₉₁-Dha (lane 2) were spotted (2×10³, 2×10² and 2×10¹ cells) on plates lacking uracil and tryptophan.

FIG. 10 Ste4p induces an altered conformation in Ste18p. A) Cells containing Nᵢₐ-STE18₉₁-C_{ub}—RURA3 and an empty vector or P_{GAL1}HA-STE4 (shown are two independent transformants) and cells containing N-terminally truncated derivatives of Nᵢₐ-STE18₉₁-C_{ub}—RURA3 and P_{GAL1}HA-STE4 were spotted (10⁵, 10³, 10², 10¹) on plates lacking uracil, tryptophan, and containing galactose to induce or glucose to repress the expression of HA-Ste4p. Plates contained 20 μM copper ions to moderately express the STE18 constructs. Cells were grown for 3 days at 30° C. B) Cells expressing the different N_{vg}-Ste18_{XY}-C_{ub}-Dha fusions were grown in glucose medium and extracted for immunoblot analysis. Proteins were detected with anti-HA antibody after SDS 12.5% PAGE. C) Schematic drawing of the STE18 constructs, Boxes indicate the position of the α-helices according to Sondek et al. (Sondek et al., Nature 379: 369-374, 1996). Numbers indicate the lengths of the constructs in amino acids. The numbers include the last three C-terminal residues of Ste18p, which are removed proteolytically during the isoprenylation of the protein.

FIG. 11 The ratio of uncleaved to cleaved N_{vg}-Ste18₉₁-C_{ub}-Dha is influenced by coexpression of HA-Ste4p. A) Cells containing N_{vg}STE18₉₁-C_{ub}Dha (lanes 1-4) or N_{vg}-STE18₇₄-C_{ub}-Dha (lanes 5-8) and HA-STE4 (lanes 2, 4, 6, 8) or an empty vector (lanes 1, 3, 5, 7) were grown in galactose to express HA-Ste4p. One hour prior to protein extraction 100 μM copper sulfate was added to one set of samples to increase the expression of the STE18 constructs (lanes 1, 2, 5, 6). Samples without addition of copper are shown in lanes 3, 4, 7, and 8. Proteins were detected by HA antibody after SDS-12.5% PAGE and transfer onto nitrocellulose. B) The ratio of uncleaved to cleaved fusion protein was calculated by quantitative chemiluminescence. Shown are the average of the values from five experiments with cells expressing N_{vg}-Ste18₉₁-C_{ub}-Dha from the uninduced P_{CUP1} promoter (lanes 3, 4), and the average of the values from three experiments with cells expressing N_{vg}-Ste18₇₄-C_{ub}-Dha from the uninduced P_{CUP1} promoter (lanes 7, 8).

FIG. 12 Measuring the effects of cancer causing mutations on the stability of the p53core in vivo. (A) N_{vg}-p53_{core}-C_{ub}-Dha and different p53_{core} mutants were expressed in yeast at 37° C. and the cleaved and uncleaved fraction of the fusion proteins were probed with the anti-HA anti-body on a nitrocellulose blot after cell extraction and SDS-PAGE. The amount of cleaved and uncleaned fusion protein were quantified by chemiluminescence. The amino acid exchanges of the different p53_{core} mutants are indicated at the top of the blot. A stretch of amino acids of the first beta-strand of the p53core is missing in Δ. (B) Representation of the effect of different mutations on the stability of the p53_{core}. For each experiment the ratio of uncleaved N_{vg}-p53_{core}-C_{ub}-Dha was calculated. This value was subtracted from the ratio of uncleaved N_{vg}-p53_{core}-C_{ub}-Dha of the different mutants of p53_{core}. A positive value, indicating a higher fraction of uncleaved fusion protein indicates destabilization of the mutant protein whereas a negative value indicating a higher fraction of cleaved Dha indicates stabilization of the p53_{core}. Experiments were performed at 30° C. (black bars) and 37° C. (grey bars). Five independent experiments at each temperature are shown except for C242S (four experiments at 37° C.) and R175H (six experiments at 37° C).

FIG. 13 Monitoring the conformational stability of the p53_{core} and the V143A mutant of the p53_{core} by a simple growth assay. (A) Western bot of protein extracts of yeast expressing N_{ub}p53_{core}-C_{ub}-Dha and N_{ub}—V143A-C_{ub}-Dha containing the N_{ub}mutants Nᵢₐ and N_{ig}. The quantification of the experiment is shown in (B). Bars indicate the percentage of cleaved Dha. (C) Growth assay of yeast cells expressing the corresponding Nᵢᵢ, Nᵢₐ, and N_{ig} N_{ub}-p53_{core}-C_{ub}—RUra3p fusion proteins on media containing 5-FOA. The Nᵢₐ fusion protein allows to distinguish between the cells expressing the wild type p53_{core} (growth) and the cells expressing the V143A mutant of the core (non-growth).

### BEST MODE FOR CARRYING OUT THE INVENTION DETAILED DESCRIPTION OF THE INVENTION

1. General

In general the invention provides methods and reagents for monitoring protein conformation by attaching N_{ub} and C_{ub} to the N- and C-terminus of the same polypeptide, thereby allowing the measurement of intramolecular N_{ub} and C_{ub} reassociation by quantifying the ratio of cleaved to uncleaved fusion protein. The resulting ratio is defined by the affinity of N_{ub} to C_{ub}, and by the nature of the polypeptide separating N_{ub} from C_{ub}. The invention further provides a variety of mutant N_{ub} sequences for varying the intrinsic affinity of the N_{ub} and C_{ub} moieties. By introducing mutations into N_{ub} and thereby altering its affinity for C_{ub}, a cleavage spectrum of different N_{ub}—X—C_{ub} fusion proteins is obtained which is characteristic for the inserted polypeptide X. Two features of the inserted protein dominate this spectrum: the position of the N- and C-termini relative to each other in the folded conformation, and the rigidity of the structure. Mutations or conditions that favor the unfolded state, or that induce a conformation which will alter the time-averaged distance between the N- and the C-terminus of tie protein, are detected by this technique. This difference will influence the rates of reassociation between the N_{ub} and C_{ub}, and as a consequence will result in different amounts of cleaved reporter moiety. Calculating the ratio of cleaved to uncleaved protein is in this assay less sensitive to varying expression levels or changes in the absolute amount of protein. However, changes in the structure that do not significantly alter the time-averaged distance between the N- and the C-terminus remain concealed.

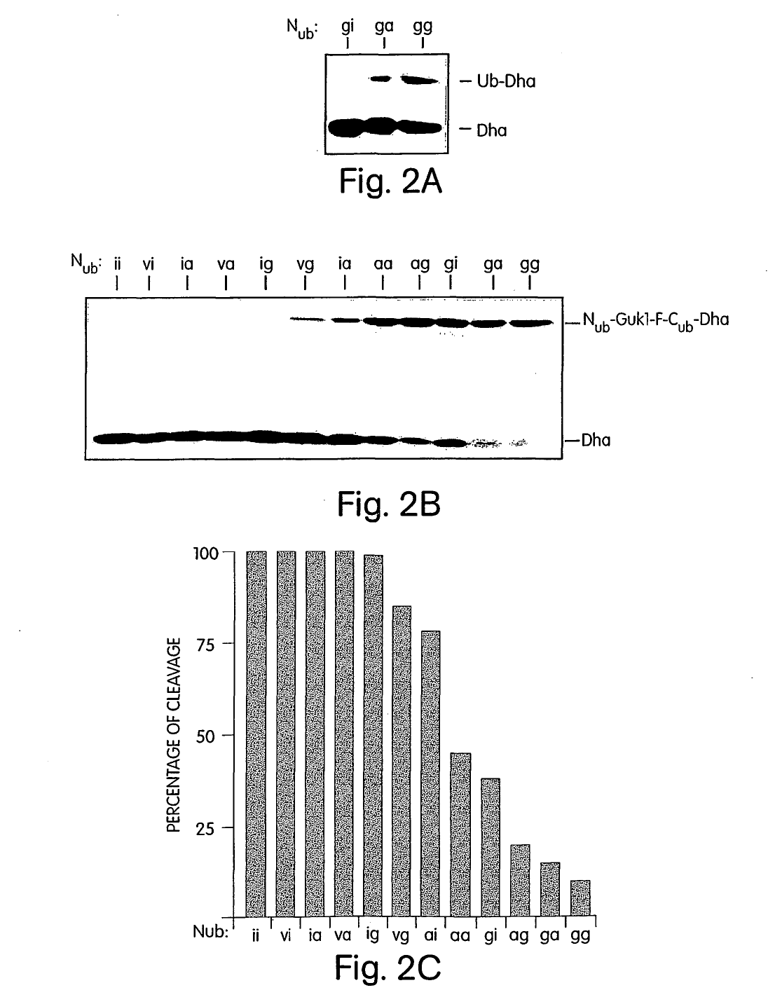

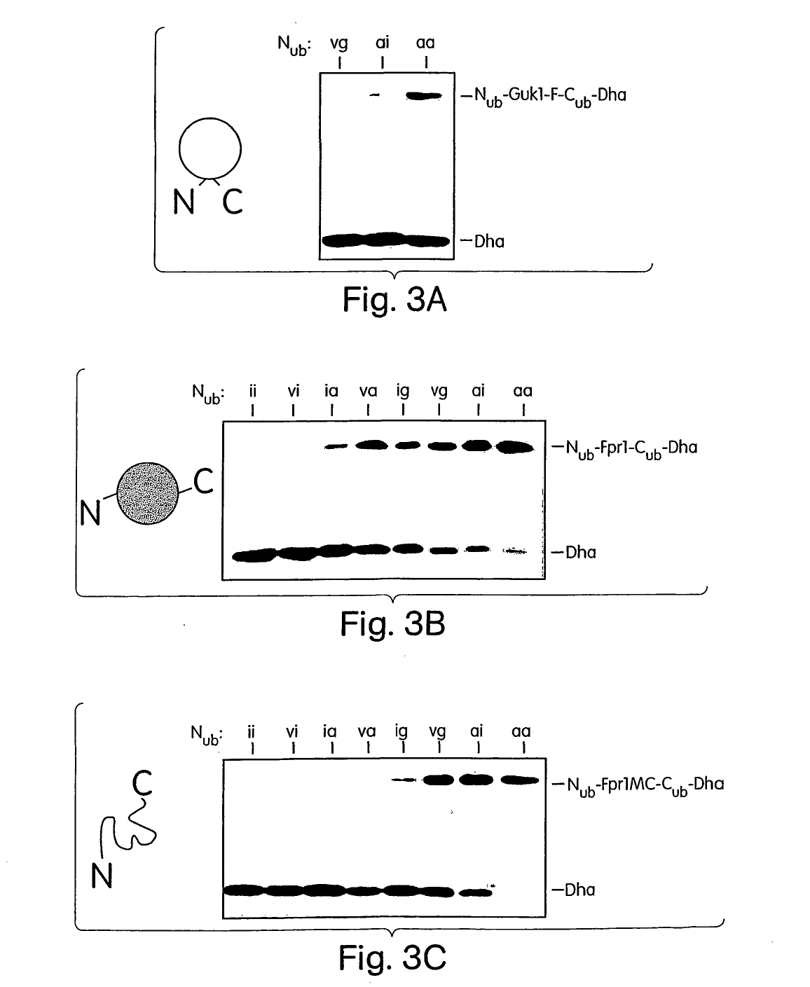

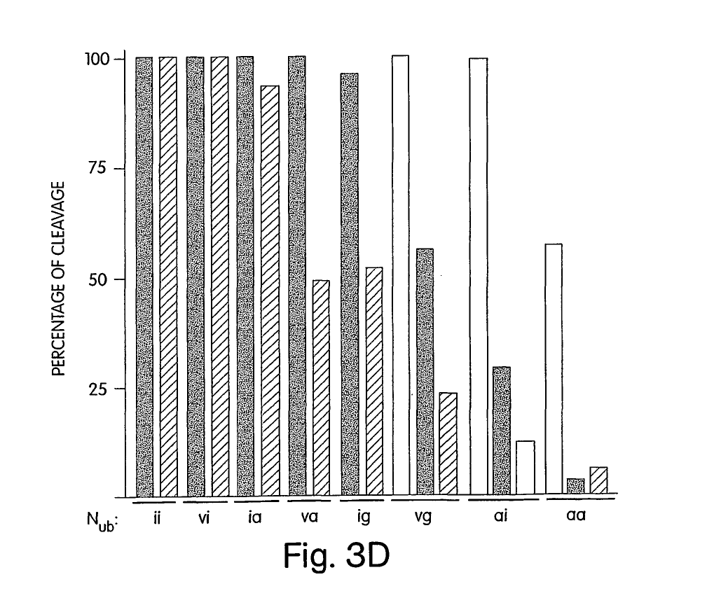

The invention allows the balance between the folded and the unfolded state of a protein to be sensitively adjusted by appropriate selection of the N_{ub} moiety. Attaching N_{ub} and C_{ub} to the termini of a protein will disturb this balance. This effect is clearly seen for the Nᵢᵢ-labeled fusion proteins where cleavage is always complete irrespective of the position of tie termini and the stability of the structure (FIGS. 2, 3). To monitor alterations in the conformation of a protein, preferably a N_{ub}—C_{ub}-pair has to be selected that does not disturb the balance between the folded and the unfolded state too much. That is, it may be advantageous that a N_{ub} is chosen such that for a given protein it just inhibits or just allows the reconstitution of a quasi-native ubiquity and hence cleavage of the reporter moiety from the fusion protein. For example, in one favoured embodiment, a N_{ub} is closen that just inhibits the growth of yeast cells expressing a N_{ub}—X—C_{ub}—RUra3p fusion protein when plated on appropriate selective medium. The detection of uncleaved fusion protein in a steady state analysis is a good first indication. However, the optimal extent of cleavage depends on the known or expected shift upon destabilizing the structure. The optimal cleavage is greater than 50% for proteins like Guk1p and less than 50% for proteins like Fpr1p.

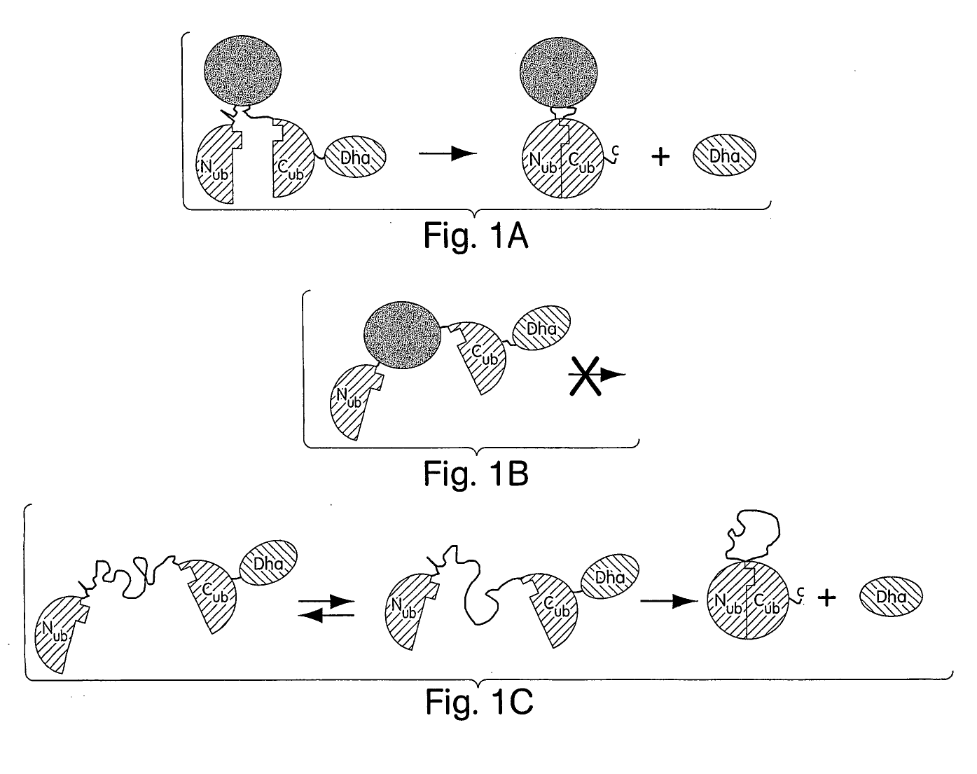

The invention provides additional functional ubiquitin mutant having particular desirable reassociation properties. The cleavage spectrum of N_{ub}-Guk1-F—C_{ub}-Dha revealed an interesting feature about the influence of the two isoleucines at position three and thirteen of Ub on the stability of the protein (FIG. 2c). The amount of cleaved Dha is always higher for the N_{ub}-fusions carrying the residue with the smaller side chain at position three of N_{ub} (compare Nᵢₐ with Nₐᵢ, N_{ig} with N_{gi} or N_{ga} with N_{ag}; FIG. 1c). We therefore conclude that the isoleucine at position three contributes more to the stability of Ub than the same residue at position 13 does. This confirms that the effect of deleting methyl groups from the hydrophobic core on the stability of a protein depends very much on the context of the affected side chain (Main et al., Biochemistry 37: 6145-53, 1998).

Therefore, one aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide selected from the group consisting of: Guk1, Fpr1, Sec62p, beta-amyloid, p53, calmodulin, estrogen receptor alpha (ERα), FKBP, G-protein, VHL, tyrosine kinase, Src, Abl, Epidermal Growth Factor Receptor (EGFR), Protein Kinase A (PKA), Protein Kinase C (PKC), Cyclophillins, Cyclin Dependent Kinases (CDKs), Cyclins, a protein of therapeutic, physiological or biological interest or variants/fragments thereof.

In another aspect of the invention, the nonubiquitin polypeptide may be a protein of therapeutic, physiological or biological interest. For example, such proteins include VHL, tyrosine kinases, Src, Abl, Epidermal Growth Factor (EGF) receptor, Protein Kinase A (PKA) Protein Kinase C (PKC), Cyclophillins, Cyclin Dependent Kinases (Cdk), Cyclins or variants/fragments thereof.

In one aspect of the invention, the nonubiquitin polypeptide comprises a protein from a non-yeast species. Such polypeptides offer particularly advantageous features and may be obtained from species that include mammalian, insect, plant, vertebrate, animal or prokaryotic species. As will be appreciated by the skilled artisan, fusion proteins of the invention comprising such polypeptides may be obtained by first isolating or synthesizing nucleic acids that encode a non-yeast polypeptide of interest and then expressing it as a fusion protein of the invention in a suitable host cell.

In preferable embodiments of the invention, cleavage of the reporter moiety can be detected. This may be conduced, for example, by using a Western blot using an antibody specific for the reporter moiety or an epitope attached to the reporter moiety. Many suitable epitopes will be known to a person skilled in the art, and include HLAD, HA, HIS etc. In other embodiments, the reporter moiety has anactvity. In yet other embodiments, the report moiety is a selectable marker, a transcription factor or a fluorescent marker. In a preferred embodiment, the selectable marker is selected from the group consisting of: URA3, HIS3, LYS2, HygTk, Tkneo, TkBSD, PACTk, HygCoda, Codaneo, CodaBSD, PACCoda, Tk, codA, HPRT, and GPT2. In a related preferred embodiment, the selectable marker is selected from the group consisting of TRP1, CYH2, and CAN1. In other embodiments, the reporter moiety is a negative selectable marker such as URA3 or CYH2. In a related embodiment, the reporter moiety is a positive selectable marker such as HIS3, TRP2, ADE2, Zeocin, Bla, β-galactosidase.

In a related aspect of the invention, there is provided a fusion protein comprising the structure N_{ub}—X—C_{ub}—RM, wherein C_{ub} is a carboxy-terminal ubiquitin domain, RM is a reporter moiety fused to the carboxy-terminus of the C_{ub} domain, X is a nonubiquitin polypeptide, and N_{ub} is a mutant amino-terminal ubiquitin domain selected from the group consisting of: Nᵥᵢ, Nᵥₐ, N_{vg}, Nₐᵢ, Nₐₐ, N_{ag}, N_{gi}, N_{ga}, and N_{gg}.

A related aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide, wherein RM is a selectable marker.

In another related aspect, there is provided a fusion protein comprising the structure N_{ub}—X—C_{ub}—RM, wherein C_{ub} is a carboxy-terminal ubiquitin domain, RM is a reporter moiety fused to the carboxy-terminus of the C_{ub} domain, X is a nonubiquitin polypeptide, and N_{ub} is a mutant amino-terminal ubiquitin domain which has altered affinity for C_{ub} in such a way that cleavage of the RM from C_{ub} as a result of reconstituting a quasi-native ubiquitin moiety leads to a growth advantage on a selective media for a cell harboring the N_{ub}—X—C_{ub}—RM fusion protein.

A related aspect of the invention provides a fusion protein comprising the structure N_{ub}—X—C_{ub}—RM, wherein C_{ub} is a carboxy-terminal ubiquitin domain, RM, is a reporter moiety fused to the carboxy-terminus of the C_{ub} domain, X is a nonubiquitin polypeptide, and N_{ub} is a mutant amino-terminal ubiquitin domain which has altered affinity for C_{ub} chosen such that for a given X polypeptide it just inhibits or just allows the reconstitution of a quasi-native ubiquitin and hence cleavage of RM from the fusion protein.

In this case, N_{ub} can be generated by any art-recognized mutagenesis procedure (such as random mutagenesis or combiniatory mutagenesis). For a predetermined X protein, the candidate plurality (or library) of N_{ub}—X—C_{ub}—RM constructs can be introduced into a plurality of target cells. The ability of any of these cells to cleaves the RM from the fusion protein depends on tie reconstitution of a quasi-native ubiquitin moiety (see-below). When a conformation change of the X protein induces a favorable contact between C_{ub} and one of the mutant N_{ub}'s, a quasi-native ubiquitin moiety may be reconstituted, resulting in the cleavage of the C_{ub}—RM juncture. In a cell that has a functional N-end rule pathway (see below), the RM may (or may not be) degraded based on the identity of its nascent N-terminal amino acid, and the survival of the host cell on a selective media can be determined by this event. For example, if RM is a negative selectable marker (R-ura3, see example below), only cells that have lost the RM will survive on the selective media (e.g. 5-FOA). Such cells can be selected and their N_{ub}—X—C_{ub}—RM constructs recovered to obtain the N_{ub} mutant. Alternatively, if RM is a transcription factor that is stable after cleavage, it may enter nucleus to initiate the transcription of a gene essential for host cell survival in a selective media—a function that a tethered RM is unable to perform because of its cytosolic or non-nucleus localization or its unfavorable conformation as a N_{ub}—X—C_{ub}—RM fusion protein. Alternatively, it may be desirable to chose a mutant Nub that just allows association of the amino and carboxy ubiquity domains of a N_{ub}—X—C_{ub}—RM fusion protein. Such mutations would be useful to detect conformational changes in the X polypeptide that cause disassociation of the quasi-native ubiquitin moiety, for example through the use of a RM that is a positive selectable marker with a non-methionine first amino acid after cleavage.

Thus, the invention also provides a method to identify a fusion protein comprising the structure N_{ub}—X—C_{ub}—RM, wherein C_{ub} is a carboxy-terminal ubiquitin domain, RM is a reporter moiety fused to the carboxy-terminus of the C_{ub} domain, X is a nonubiquitin protein, and N_{ub} is a mutant amino-terminal ubiquitin domain which has altered affinity for C_{ub} in such a way that the cleavage of the RM from C_{ub} as a result of reconstituting a quasi-native ubiquitin moiety leads to a growth advantage of a cell harboring the N_{ub}—X—C_{ub}—RM fusion protein to grow on a selective media, comprising: (i) generating a plurality of mutant N_{ub} of the N_{ub}—X—C_{ub}—RM fusion construct; (ii) introducing the plurality of mutant N_{ub}—X—C_{ub}—RM fusion constructs into a plurality of host cells; (iii) causing the conformation change of the X protein; (iv) selecting for at least one surviving cell on the selective media; and, (v) identifying the fusion protein encoded by the N_{ub}—X—C_{ub}RM fusion construct in the survival cell.

A related aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a non-yeast nonubiquitin polypeptide.

A related aspect of the invention provides a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is a reporter moiety fused to the carboxy-terminus of the C_{ub} domain, wherein upon cleavage of the C_{ub}—RM junction, the first amino acid of the released RM is an amino acid other than methionine.

In one embodiment, the first amino acid of the cleaved RM is Arginine, Lysine, Histidine, Phenylalanine, Tryptophan, Tyrosine, Leucine, Aspartate, Glutamate, Cysteine, Asparagine, Glutamine or Isoleucine.

Another aspect of the invention provides a polynucleotide sequence encoding any one of the fusion proteins of the instant invention.

In another aspect of the invention, there is provided a vector that encompasses a fusion polynucleotide encoding any of the fusion proteins of the instant invention.

In another aspect of the invention, there is provided a host cell that harbors a vector or a fusion polynucleotide or a fusion protein of the instant invention. Various types of host cells will be suitable to practice this aspect of the invention and include a mammalian cell, or a plant cell, or an insect cell. In certain embodiments, the cell is selected from the group consisting of: a human cell, a mouse cell, a rat cell, a hamster cell, a zebrafish cell, a Drosophila cell, or a nematode cell. In another embodiment, the cell is selected from the group consisting of: an A. thaliana cell and an N. tabacum cell.

The invention further provides methods for measuring the effects of a number of stimuli on polypeptide conformation.

One aspect of the invention provides a method of detecting a conformational change in a polypeptide resulting from a mutational alternation polypeptide sequence comprising: (a) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety, wherein upon cleavage of the C_{ub}—RM junction, the first amino acid of the release RM is an amino acid other than methionine; and, (b) measuring a second fusion protein reporter moiety activity from a N_{ub}—X′—C_{ub}—RM, wherein X′ is a Mutationally altered form of polypeptide X; wherein a change in the level of the second fusion protein RM activity relative to the first fusion protein RM activity indicates that the polypeptide has undergone a conformation change resulting from the mutational alteration.

In a related aspect of the invention, there is provided a method of detecting a conformational change in a polypeptide resulting from a point mutation or a small insertion or deletion (such as an insertion/deletion of no more than 3 amino aicds, preferably no more than 5, 10, 15, 20, 30, or 50 amino acids) in the polypeptide sequence comprising: (a) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety; and, (b) measuring a second fusion protein reporter moiety activity from a N_{ub}—X′-C_{ub}-RM, wherein X′ is a point mutation or a deletion/insertion of no more than three amino acids form of polypeptide X; wherein a change in the level of the second fusion protein RM activity relative to the first fusion protein RM activity indicates that the polypeptide has undergone a conformation change resulting from the mutational alteration.

For example, as shown in Example 3, there are a number of p53 point mutations that will cause a conformation change of the p53 core domain. Point mutation includes one or more point mutations in the same protein, either adjacent to one another or apart from one another in the primary protein sequence.

In one embodiment, the mutation is selected from deletion, insertion/addition, substitution, reversion, missense or nonsense point mutation.

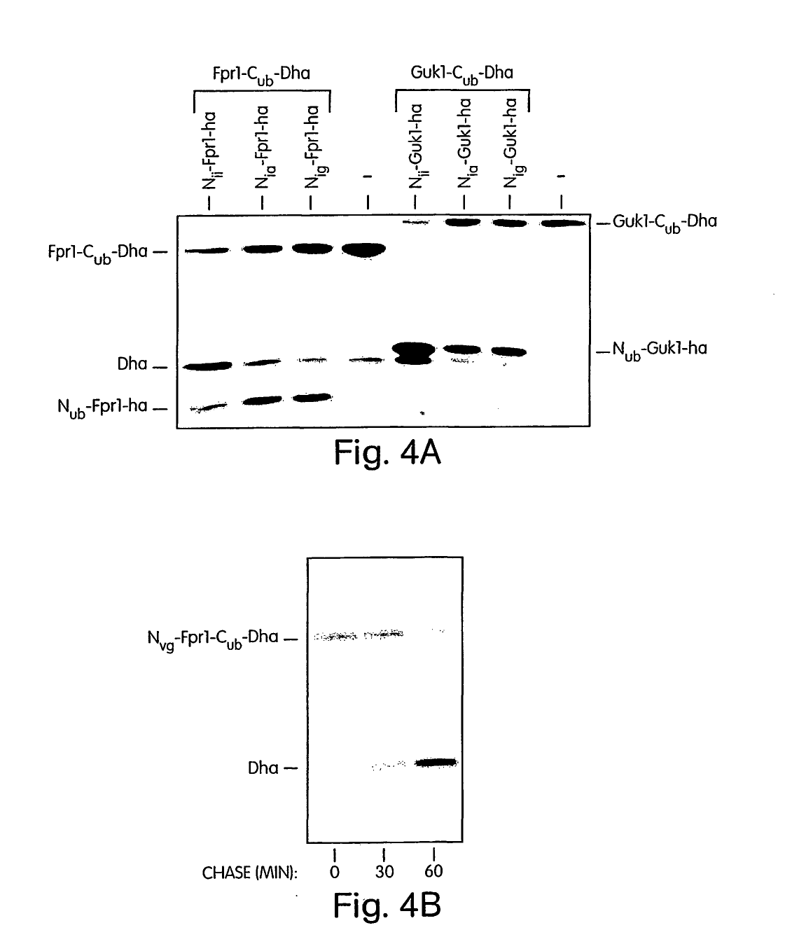

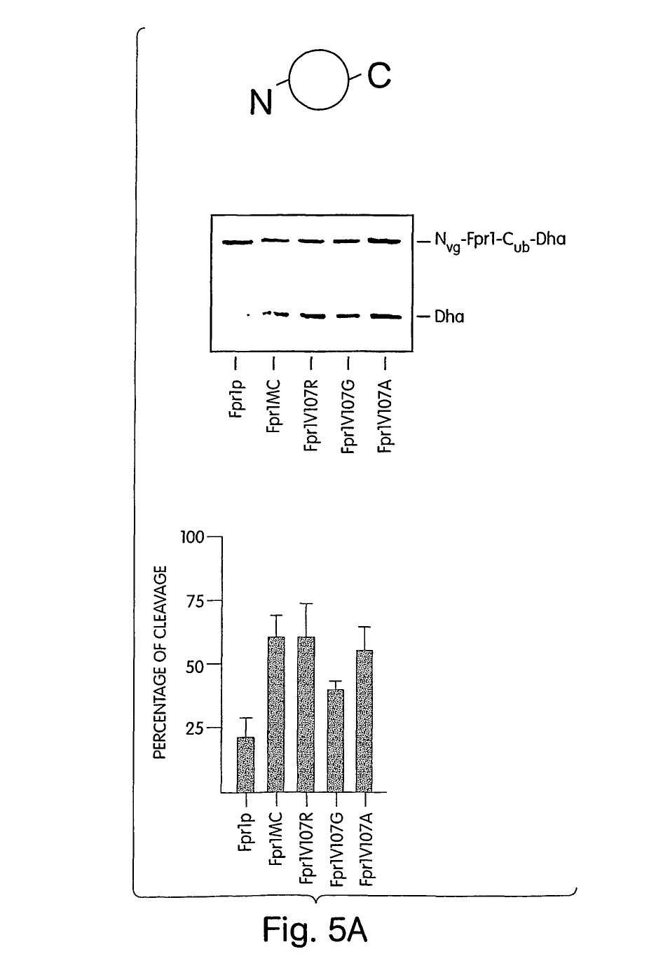

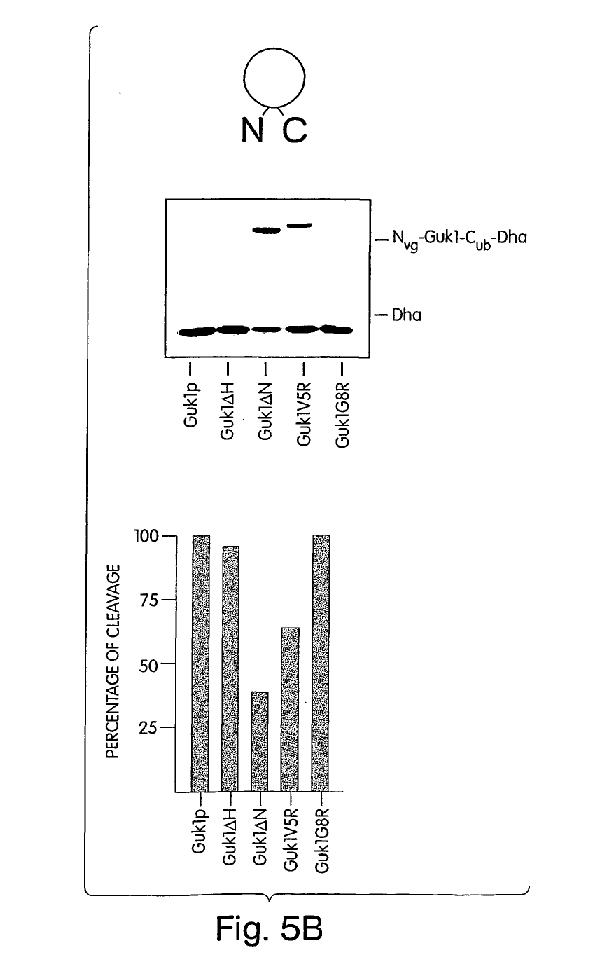

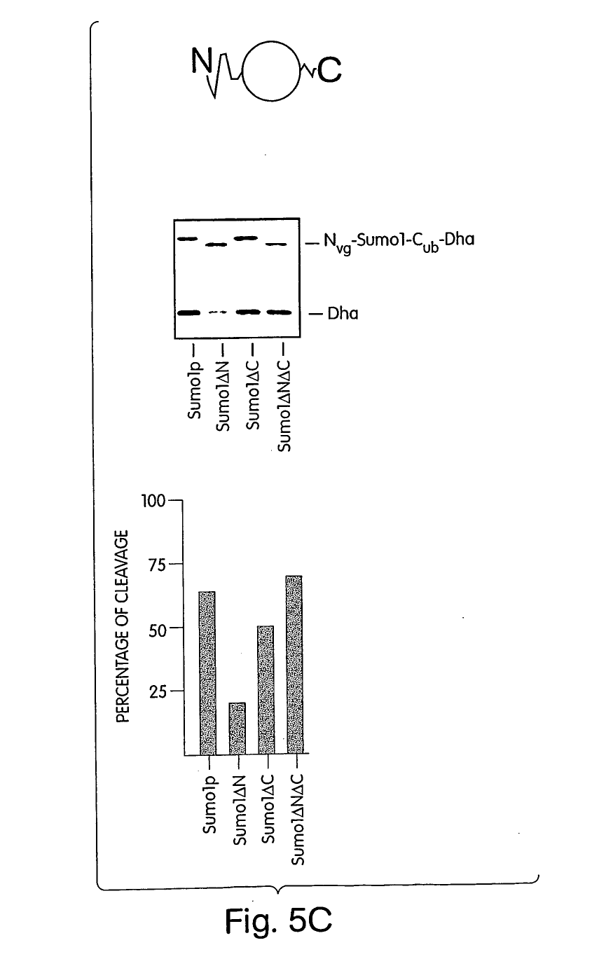

For example, the technique was used to measure the effect of mutations in Fpr1p which we assumed would most probably alter the general stability of the protein. This was achieved by drastically changing residues in the C-terminal-strand of the protein. Consequently, the amount of the cleaved N_{vg}—C_{ub}-fusion protein rose from 22% for the wild type to approximately 61% for the mutant. By replacing the valine at position 107 that is part of the C-terminal-strand with alanine or glycine, and comparing the efficiency of N_{ub}—C_{ub} reassembly in the corresponding constructs with the native and the probably unfolded protein (Fpr1MC), we could measure the contributions of the deleted methyl groups to the stability of the protein. Compared to the native protein, the alanine mutations shift the ratio of cleaved to uncleaved N_{vg}-Fpr1-C_{ub}-Dha from 22% to 55% (FIG. 4a). The corresponding mutation in the human FKBP12 has been previously analyzed in vitro. The structure of FKBP12 is superimposable onto the structure of the yeast homologue Fpr1p (Rotonda et al., J. Biol. Chem. 268: 7607-09, 1993). Replacing the equivalent valine 101 with an alanine reduces the stability of the human protein by 2.75 kcalmol⁻¹ (Main et al., Biochemistry 37: 6145-53, 1998). In these experiments, FKBP12 was denatured by urea and the unfolding was followed by changes in the spectroscopic parameters of the protein ensemble. The split-Ub assisted analysis was performed under cellular conditions at 30° C. We inferred that the increase in cleavage, which is detected for the mutant protein, is caused by a higher proportion of unfolded chains which permits the N_{ub}—C_{ub} reassociation. We also assumed that the extent of unfolding correlates with the extent of cleavage. A glycine at this position of the protein should therefore destabilize the structure of Fpr1p even more than the corresponding alanine exchange (Kellis et al., Biochemistry 28: 4914-22, 1989). Instead of observing a further increase, we detected a decrease in the efficiency of cleavage compared to the alanine mutant, although the fraction of cleaved reporter is still twofold higher than for the wild type protein (FIG. 5a). This unexpected outcome might reveal the oversimplification of our assumption that conformations of different unfolded proteins or mutants are exchangeable. Especially under physiological conditions, unfolding might lead to misfolding or even aggregation. We therefore propose that the glycine mutation not only shifts the equilibrium towards more unfolded chains, but might also favor an ensemble of unfolded conformations that impedes the N_{ub}—C_{ub} reassociation more than the protein carrying the alanine in this position. How much of this effect can be attributed to the cellular environment in which these measurements were performed is an interesting question that can only be answered once a more detailed biochemical analysis or even the solution structures of the two mutants are achieved (Logan et al., J. Mol. Biol. 236: 637-48, 1994).

A related aspect of the invention provides a method of detecting a conformational change in a polypeptide resulting from a stimulus comprising: (a) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety; and, (b) measuring a second fusion protein reporter moiety activity from a N_{ub}—X′—C_{ub}—RM, wherein X′ is the X polypeptide which has been altered by the stimulus; wherein a change in the level of the second fusion protein RM activity relative to the first fusion protein RM activity indicates that the polypeptide has undergone a conformation change resulting from the stimulus.

In a preferred embodiment, the stimulus is an alteration in environmental factor, which can be pH change, temperature change, pressure change, redox-state change or ionic strength change.

In another preferred embodiment, the stimulus ia a post-translational modification of the X protein, which can be phosphorylation, methylation, prenylation, acetylation, palmitoylation, myristoylation, reduction, oxidation, glycosylation, proteolytic cleavage, sulfation, hydroxylation, carboxylation, or the covalent linkage of ubiquitin-like proteins (Ubl) to X such as ubiquitination or sumoylation. In a related embodiment, the post-translational modification may be brought about through the use of a test compound or an alteration in an environmental factor. For example, phophorylation through the provision of a protein kinase, or the reduction of a protein through a change in redox state or through the provision of a reducing agent.

In another preferred embodiment, the stimulus is contacting the X protein with a test compound in trans, which test compound is selected from the group consisting of: a polypeptide, a hormone, a steroid, an ion, a polynucleotide, an oligosaccharide, a lipid, an enzyme substrate, gas molecules such as CO, CO₂ or O₂, a small molecule, a co-factor, a vitamin, a metal ion, and a nucleotide phosphate. In certain embodiments, for example where the test compound is a polypeptide, the test compound may be provided by the expression of a nucleotide sequence. A test polypeptide, for example, may be provided by any of numerous methods known to the skilled artisan including recombinant DNA technology, expression cloning, from a cDNA or genomic library. Alternatively, test compounds may be provided by the expression of activity of endogenous or recombinant genes, such as through the use of recombinant or natural organisms that produce proteins or secondary metabolites suitable for testing.

In another preferred embodiment, the nonubiquitin polypeptide of interest is selected from the group consisting of: Guk1, Fpr1, Sec62p, beta-amyloid, p53, calmodulin, estrogen receptor alpha (ERα), FKBP, and G-protein, VHL, tyrosine kinases, Src, Abl, Epidermal Growth Factor (EGF) receptor, Protein Kinase A (PKA) Protein Kinase C (PKC), Cyclophillins, Cyclin Dependent Kinases (Cdk), Cyclins, a protein of therapeutic, physiological or biological interest or variants/fragments thereof.

In another preferred embodiment, the N_{ub} domain is an amino-terminal ubiquitin domain or a mutant amino-terminal ubiquitin domain selected from the group consisting of: Nᵢₐ, N_{ig}, Nᵥᵢ, Nᵥₐ, N_{vg}, Nₐᵢ, Nₐₐ, N_{ag}, N_{gi}, N_{ga}, and N_{gg}.

In another preferred embodiment, the reporter moiety (RM) is a selectable marker.

In another preferred embodiment, the first amino acid of the RM is a non-methionine residue when the RM is released by cleavage of the C_{ub}—RM junction by a ubiquitin-specific protease (UBP).

In another preferred embodiment, N_{ub} is a mutant amino-terminal ubiquitin domain winch has altered affinity for C_{ub} chosen such that for a given X polypeptide it just inhibits or just allows the reconstitution of a quasi-native ubiquitin and hence cleavage of RM from the fusion protein.

In another preferred embodiment, X is a non-yeast nonubiquitin polypeptide.

In certain preferred embodiment, at least one step is performed in a host cell expressing a ubiquitin specific protease. In such embodiments, it may be sufficient that at least step of a method be practiced within a host cell. A skilled artisan will be able to easily determine, from a large number of appropriate host cell types, which type will be appropriate for practicing a given step of a method. In preferred embodiments, the host cell will express a ubiquity-specific protease. In an alternative-embodiment, a cell-free environment may be utilised to practice the methods of the invention. For example, a cell-extract, a transcription-translation mixture or an appropriate set of purified proteins in an appropriate buffer. In certain embodiments, it is preferred that the cell-free environment contains a ubiquitin-specific protease and/or components of the N-end rule protein degregation system.

Another aspect of the invention provides a method for detecting a conformation change of a protein resulting from a stimulus, comprising: (a) measuring a first spectrum of fusion protein reporter moiety activity from a first panel of at least two fusion proteins, each different from the other, comprising the general structure N_{ub}—X—C_{ub}—RM, wherein N_{ub} is an amino-terminal ubiquitin domain or mutant amino-terminal ubiquitin domain selected from at least one of Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, and N_{gg}, C_{ub} is a carboxy-terminal ubiquitin domain, X is the protein, and RM is a reporter moiety; (b) measuring a second spectrum of fusion protein reporter moiety activity from a second panel of fusion proteins comprising the general structure N_{ub}—X′—C_{ub}—RM, wherein the second panel of N_{ub} and C_{ub} fusion proteins are the same as the first panel of N_{ub} and C_{ub} fusion proteins, X′ is the X protein resulting from treating the X protein with the stimulus, and RM is a reporter moiety; (c) comparing the first and second spectra of fusion protein reporter moiety activity; wherein a shift in the spectrum of reporter moiety activity indicates that the protein has undergone a conformation change resulting from the stimulus.

In a preferred embodiment, the stimulus is a mutational alteration of the X protein, an alteration in environmental factor, a post-translational modification of the X protein, or contacting the X protein with a test compound in trans.

In related embodiments, other stimuli may be detected by the selection of an appropriate X protein, or alternatively, a given X protein may change conformation with another stimulus or in fact more than one stimulus.

The invention further provides a number of methods to identify or screen for conditions that may cause a conformation change of a protein.

One aspect of the invention provides a method to identify a compound which can change the conformation of a protein upon contacting the protein, comprising: (a) providing a plurality of test compounds which are not known to be able to cause the conformation change of the protein; (b) testing each compound by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin protein of interest, and RM is a reporter moiety; and, measuring a second fusion protein reporter moiety activity from a N_{ub}—X′—C_{ub}—RM, wherein X′ is the X protein which has been altered by the test compound; wherein a change in the level of the second fusion protein RM activity relative to the first fusion protein RM activity indicates that the X protein has undergone a conformation change resulting from contacting the test compound, thereby identifying a compound which can change the conformation of the protein.

In a preferred embodiment, the method further comprises formulating the identified compound into a pharmaceutical composition.

In another preferred embodiment, the plurality of test compounds is a library of compounds which comprises 2 to 10 test compounds, or greater than 10 test compounds. Preferably 10 to 500, 500 to 10,000 or greater than 10,000 test compounds.

Another aspect of the invention provides a method to identify a mutation in a protein which leads to the conformation change of the protein, comprising: (a) generating a plurality of candidate mutations of the protein; (b) testing each candidate mutation by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin protein of interest and RM is a reporter moiety; and, measuring a second fusion protein reporter moiety activity from a N_{ub}—X′—C_{ub}—RM, wherein X′ is a mutational altered form of the X protein harboring the candidate mutation; wherein a change in the level of the second fusion protein RM activity relative to the first fusion protein RM activity indicates that the X protein has undergone a conformation change resulting from the candidate mutation, thereby identifying a mutation which can change the conformation of the protein.

Another aspect of the invention provides a method to identify a protein which changes conformation upon contacting a given compound or encountering an alteration in environmental factor, comprising: (a) providing a plurality of test proteins; (b) testing each protein X by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is the nonubiquitin test protein, and RM is a reporter moiety; and, measuring a second fusion protein reporter moiety activity from a N_{ub}—X′—C_{ub}—RM, wherein X′ is the X protein which has been altered by contacting the given compound or by the given alteration in environmental factor; wherein a change in the level of the second fusion protein RM activity relative to the first fusion protein RM activity indicates that the X protein has undergone a conformation change resulting from the given alteration in environmental factor or contacting the given compound, thereby identifying a protein which changes conformation upon contacting a given compound or encountering an alteration in environmental factors.

In certain preferred embodiments, test compounds, mutations of X proteins may be provided as a plurality of test compounds, mutations of X proteins wherein the plurality is a library. As will be obvious to a person skilled in the art, such libraries may be provided by a variety of chemical, biochemical, natural or genetic means (see below). Such libraries may comprise 2 to 10, 10 to 500, 500 to 10,000 or greater than 10,000 members.

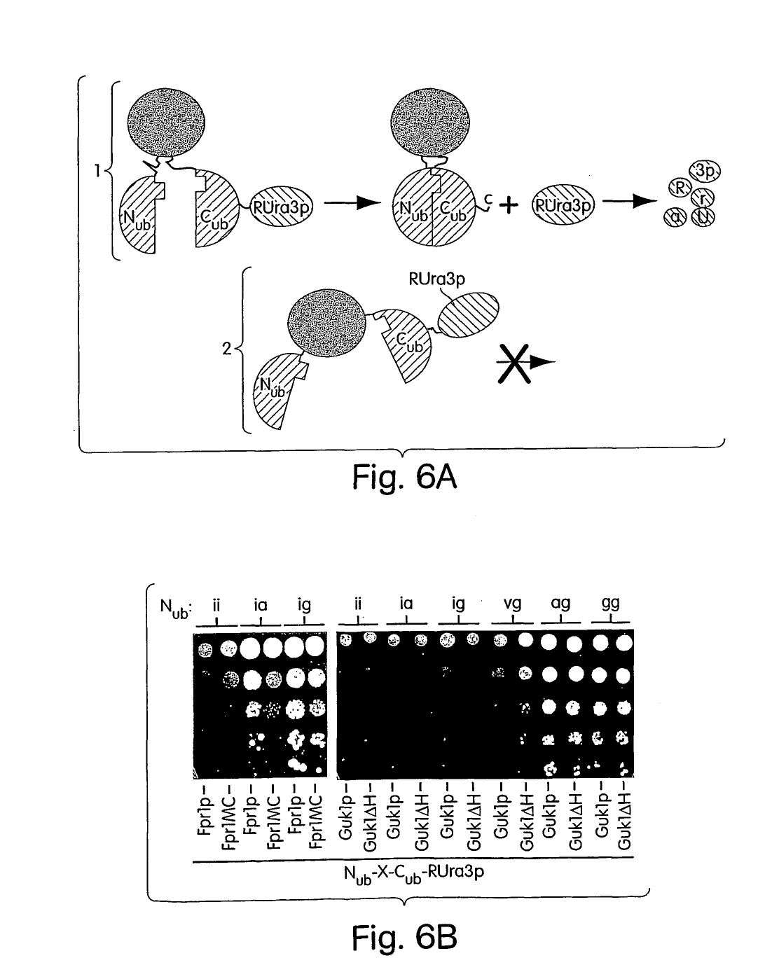

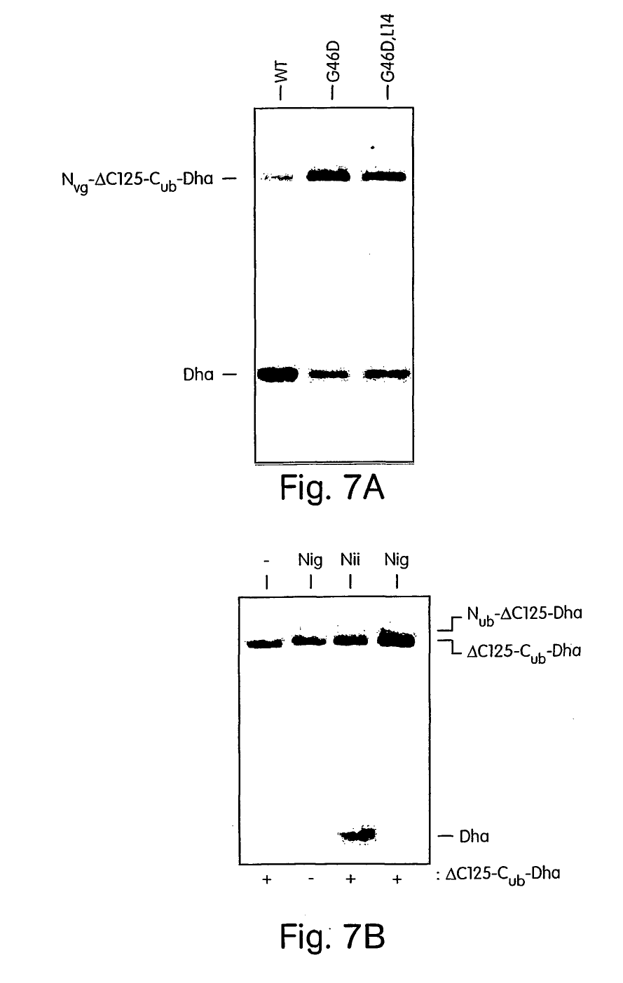

The invention further provides a useful assay for monitoring the effect of specific amino acid alterations on protein conformation. Using this assay, we learned that the mutation of an intensively studied allele of SEC62 most likely induces a conformational alteration in the N-terminal cytosolic domain of this protein (FIG. 7). Since the exchange of the glycine for an aspartate at position 46 increases the proportion of uncleaved N_{vg}-ΔC125-C_{ub}-Dha, the N-terminal domain of Sec62p reacts to the destabilization of its structure as Guk1p does (FIG. 5b). We therefore interpret the increase in mean distance between its N- and C-terminus as a reflection of a higher proportion of unfolded molecules. As a consequence, the known impaired interaction of sec62-1p with the Sec-complex will be an indirect result of the substantial increase in the proportion of molecules that are unfolded and therefore can not bind. However other interpretations might also apply. We can not exclude that the glycine exchange at position 46 is both a contact and a structural mutation, or that this mutation decreases the flexibility between the N- and the C-terminus of the N-terminal domain of Sec62p (Otzen et al., Protein Eng. 12: 41-45, 1999). Independent of the exact nature of the conformational change, the elucidation of the effect of this mutation highlights one of the potential applications of this technique. The cell-viability based variation of this assay (FIG. 6) might allow not only to discover the effect of a certain mutation in a protein, but also to search for compounds that stabilize the structure of the mutated protein.

The invention also provides various methods to conduct a pharmaceutical business.

One aspect of the invention provides a method to conduct a business, comprising: (a) by a suitable method of the invention, identifying one or more compounds which change the conformation of a polypeptide; (b) conducting therapeutic profiling of said identified compounds, or other derivatives thereof, for using the compounds in therapy for a condition; and, (c) formulating a pharmaceutical preparation including one or more compounds identified in (b) as a product having an acceptable therapeutic profile.

In a preferred embodiment, the business method further comprises establishing a distribution system for distributing said product for sale.

In another preferred embodiment, the business method further includes establishing a sales group for marketing the product.

Another aspect of the invention provides a method to conduct a business, comprising: (a) by a suitable method of the invention, identifying one or more compounds which change the conformation of a polypeptide; (b) conducting therapeutic profiling of said identified compounds, or other derivatives thereof, for using the compounds in therapy for a condition; and, (c) licensing, to a third party, the rights for further development of compounds and/or formulating a pharmaceutical preparation including one or more compounds identified in (b) to affect conformation change of the polypeptide for treatment of the condition.

Another aspect of the invention provides a method to conduct a business, comprising: (a) by one or more suitable methods of the invention, generating information or data, or identifying compounds, proteins or mutations/variants/derivatives thereof; (b) licensing, selling, providing for consideration or access to said information, said data, said identified compounds, proteins or mutations/variants/derivatives thereof.

Another aspect of the invention provides a kit for detecting or identifying alterations in the conformation of an X protein, comprising a panel of at least two vector constructs for expressing fusion proteins of the general structure N_{ub}—X—C_{ub}—RM, wherein each vector construct comprises a coding sequence for N_{ub}, an amino-terminal ubiquitin domain or a mutant amino-terminal ubiquitin domain selected from Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, or N_{gg}; a coding sequence for C_{ub}, a carboxy-terminal ubiquitin domain; a coding sequences for RM, a reporter moiety fused to the carboxy-terminus of the C_{ub} domain; and at least one cloning site or multicloning site for subcloning the X-protein in frame with both the N-terminal N_{ub} and the C-terminal C_{ub}—RM moieties; and wherein at least one vector construct expresses a mutant N_{ub} fusion protein.

In one embodiment, the kit further comprises a host cell for expressing said fusion proteins from said vector constructs.

In one embodiment, the kit further comprises instructions for detecting or identifying alterations in protein conformation by using the vector constructs.

In a related embodiment, the kit further comprises a control protein that can be subcloned into the multicloning site so that a user can positively control for a working experimental condition.

Another aspect of the invention provides a kit for measuring or detecting protein conformation change caused by a stimulus, comprising: (a) one or more vector constructs for expressing fusion proteins of the general structure N_{ub}—X—C_{ub}—RM, wherein each vector construct comprises a coding sequence for N_{ub}, an amino-terminal ubiquitin domain or a mutant amino-terminal ubiquitin domain selected from Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, or N_{gg}; a coding sequence for C_{ub}, a carboxy-terminal ubiquitin domain; a coding sequences for RM, a reporter moiety fused to the carboxy-terminus of the C_{ub} domain, and at least one cloning site or multicloning site for subcloning the X-protein in-frame with both the N-terminal N_{ub} and the C-terminal C_{ub}—RM moieties; and wherein at least one vector construct expresses a mutant N_{ub} fusion protein; (b) an instruction for using the vector construct of (a) to measure/detect protein conformation change caused by the stimulus.

In a preferred embodiment, the instruction is not physically associated with the vector constructs of (a). For example, the instruction can be posted on a website, or updated periodically, or accessible as a published document.

In another preferred embodiment, the stimulus is an alteration in environmental factor, a post-translational modification of the X protein, or contacting the X protein with a test compound in trans.

Another aspect of the invention provides a composition comprising: (a) a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain and X is a nonubiquitin polypeptide; and, (b) a compound that when brought in to contact with, causes conformational change in polypeptide X; and/or, (c) a fusion protein comprising the structure N_{ub}—X′—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain and X′ is the X nonubiquitin polypeptide which has been altered by a stimulus.

Another aspect of the invention provides a method of controlling activity of a target gene, comprising: (a) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is a reporter moiety fused to the carboxy terminus of the C_{ub} domain, wherein the reporter moiety is a gene activating moiety; (b) treating the X polypeptide with a stimulus, thereby causing the cleavage of the RM as a result of a conformational change of the X polypeptide; wherein the released RM controls activity of the target gene.

Another aspect of the invention provides a method of controlling activity of a protein, comprising: (a) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is the protein fused to the carboxy-terminus of the C_{ub} domain, wherein upon cleavage of the C_{ub}—RM junction, the first amino acid of the released RM is an amino acid other than methionine; (b) treating the X polypeptide with a stimulus, thereby causing the cleavage of the RM as a result of a conformational change of the X polypeptide; wherein the released RM is degraded by N-end rule components, thereby controlling activity of the protein.

In both of these two situations where a gene or a protein activity is to be controlled, the cleavage of the reporter moiety serves as a “switch” that can be controlled by a stimulus.

In a preferred embodiment, the stimulus is an alteration in environmental factor, a post-translational modification of the X protein, or contacting the X protein with a test compound in trans.

Another aspect of the invention provides a method to detect or measure an alteration of an environmental factor or the presence of a compound in a sample comprising the steps: (a) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide which changes confirmation from said alteration in environmental factor or presence of said compound; (b) contacting the fusion protein with the environment or the sample containing the compound; and, (c) measuring the degree of cleavage of the reporter moiety (RM) from the fusion protein; wherein a change in the degree of RM activity compared to a standard or control indicates an alternation in said environmental factor or the presence of said compound in the sample.

2. Definitions

The term “agonist”, as used herein, is meant to refer to an agent that mimics or upregulates (e.g. potentiates or supplements) bioactivity of a protein of interest, or an agent that facilitates or promotes (e.g. potentiates or supplements) an interaction among polypeptides or between a polypeptide and another molecule (e.g. a steroid, hormone, nucleic acids, small molecule etc.). An agonist can be a wild-type protein or derivative thereof having at least one bioactivity of the wild-type protein. An agonist can also be a small molecule that upregulates expression of a gene or which increases at least one bioactivity of a protein. An agonist can also be a protein or small molecule which increases the interaction of a polypeptide of interest with another molecule, e.g., a target peptide or nucleic acid.

“Antagonist” as used herein is meant to refer to an agent that downregulates (e.g. suppresses or inhibits) bioactivity of the protein of interest, or an agent that inhibits/suppresses or reduces (e.g. destabilizes or decreases) interaction among polypeptides or other molecules (e.g. steroids, hormones, nucleic acids, etc.). An antagonist can be a compound which inhibits or decreases the interaction between a protein and another molecule, e.g., a target peptide, such as interaction between ubiquitin and its substrate. An antagonist can also be a compound that downregulates expression of a gene of interest or which reduces the amount of the wild type protein present. An agonist can also be a protein or small molecule which decreases or inhibits the interaction of a polypeptide of interest with another molecule, e.g., a target peptide or nucleic acid.

“Alter” as used in “altered by a test composition” or “altered by an environmental alteration” means a number of situations in its broadest sense. It should be understood to encompass reversibly and irreversibly change caused by a test composition. In the irreversible change situation, a test compound may contact an X protein and reacts with it (for example, transferring at least part of the compound to the X protein), causing an irreversible conformation change of the X protein. Certain suicide substrate of an enzyme may react with the catalytic site of the enzyme and lead to the inactivation and irreversible conformation change of the enzyme. On the other hand, reversible change may be caused by binding of a test compound to the X protein. The test compound may or may not need to remain bound by the X protein for the X protein to assume the changed conformation for at least a certain period of time.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide, or several nucleotides, and can include substitutions, deletions, and/or insertions of nucleotides. An allele of a gene can also be a form of a gene containing mutations.

As used herein the term “bioactive fragment of a polypeptide” refers to a fragment of a full-length polypeptide, wherein the fragment specifically agonizes (mimics) or antagonizes (inhibits) the activity of a wild-type polypeptide. The bioactive fragment preferably is a fragment capable of interacting with at least one other molecule, protein or DNA, with which a full length protein can bind.

“Biological activity” or “bioactivity” or “activity” or “biological function”, which are used interchangeably, for the purposes herein means a catalytic, effector, antigenic, molecular tagging or molecular interaction function that is directly or indirectly performed by the polypeptides of this invention (whether in its native or denatured conformation), or by any subsequence thereof.

“Activity” as used iii “reporter moiety activity” means a number of things in its broadest sense. It generally means a detectable event. For example, it means an enzymatic activity if the RM is an enzyme; it means fluorescent signal if the RM is a fluorescent protein; it means a cleavage between the C_{ub} and RM if the RM is to be detected by Western blot using an antibody specific for the RM or an epitope attached to the RM (FLAG tag or HA tag, etc.); it means a cleaved (rather than a C_{ub} fusion tethered outside the nucleus) thus activated transcription factor for initiating downstream reporter gene transcription in the nucleus if the RM is a transcription factor, etc.

“Reporter moiety cleavage” means cleavage by ubiquitin-specific proteases (UBPs) of the C_{ub}-RM juncture. There could be a number of consequences resulting from this cleavage. It generally creates a detectable event. For example, it can result in a change of enzymatic activity if the RM is an enzyme, since the cleaved RM may not be stable at the presence of the N-end rule components (or it mat be stable and activated upon cleavage, due to the removal of the inhibitory N-terminal domain); it can result in a change in fluorescent signal if the RM is a fluorescent protein (for example, fluorescent RM may be degraded by N-end rule components); it can result in a detectable size change of the RM on Western blot using an antibody specific for the RM or an epitope attached to the RM (FLAG tag or HA tag, etc.); it can release a cleaved (rather than a C_{ub} fusion tethered outside the nucleus) thus activated transcription factor for initiating downstream reporter gene transcription in the nucleus if the RM is a transcription factor, etc.

“Cells,” “host cells” or “recombinant host cells” are terms used interchangeably herein. It is understood that such terms refer not only to a particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “cell death” or “necrosis”, is a phenomenon when cells die as a result of being killed by a toxic material, or other extrinsically imposed loss of function of a particular essential gene function.

“Characterize” as used herein means a detailed study of a polypeptide or a nucleic acid (polynucleotide) encoding a polypeptide to reveal relevant chemical and biological information. This information generally includes one or more, but is not limited to, the following: sequence information for protein and nucleic acid, secondary, tertiary, and quarternary structure information, molecular weight, enzymatic or other activity, isoelectric focusing point, binding affinity to other molecules, binding partners, stability, expression pattern, tissue distribution, subcellular localization, expression regulation, developmental roles, phenotypes of transgenic animals overexpressing or devoid of the polypeptide or nucleic acid, size of nucleic acid, and hybridization property of nucleic acid. A variety of standard cell and molecular biology protocols and methodologies can be used, such as gel electrophoresis, capillary electrophoresis, cloning, restriction enzyme digestion, expression profiling by hybridization, affinity chromatography, HPLC, isoelectric focusing, mass spectrometry, automated sequencing, and the generation of transgenic animals, the details of which can be found in many standard molecular biology laboratory manuals (see below). Techniques employing the hybridization of nucleic acids may, for example, utilize arrayed libraries of nucleic acids, such as oligonucleotides, cDNA or others (See, for example, U.S. Pat. No. 5,837,832)

A “chimeric polypeptide” or “fusion polypeptide” is a fusion of a first amino acid sequence encoding a first polypeptide with a second amino acid sequence defining a domain (e.g. polypeptide portion) foreign to and not substantially homologous with any domain of the first polypeptide. Such second amino acid sequence may present a domain which is found (albeit in a different polypeptide) in an organism which also expresses the first polypeptide, or it may be an “interspecies”, “intergenic”, etc. fusion of polypeptide structures expressed by different kinds of organisms. At least one of the first and the second polypeptides may also be partially or completely synthetic or random, i.e. not previously identified in any organism.

“To clone” as used herein, as will be apparent to skilled artisan, may be meant as obtaining exact copies of a given polynucleotide molecule using recombinant DNA technology. Details of molecular cloning can be found in a number of commonly used laboratory protocol books such as Molecular Cloning: A Laboratory Manual, 2nd Ed., ed. by Sambrook, Fritsch and Maniatis (Cold Spring Harbor Laboratory Press: 1989).

“To clone” as used herein, as will be apparent to skilled artisan, may be also meant as obtaining identical or nearly identical population of cells possessing a common given property, such as the presence or absence of a fluorescent marker, or a positive or negative selectable marker. The population of identical or nearly identical cells obtained by cloning is also called a “clone.” Cell cloning methods are well known in the art as described ill many commonly available laboratory manuls (see Current Protocols in Cell Biology, CD-ROM Edition, ed. by Juan S. Bonifacino, Jennifer Lippincott-Schwartz, Joe B. Harford, and Kenneth M. Yamada, John Wiley & Sons, 1999).

“Complementation screen” as used herein means genetic screening for genes or source DNA that can conferred certain specified phenotype which will not exist without the presence of said genes or source DNA. It is usually done in vivo, by introducing into cells lacking certain phenotype a library of source DNA to be screened for, and identifying cells that have obtained a source DNA and now exhibit the specified phenotype. Alternatively, it could be done in vivo by randomly inactivating genes in the genome of the cell lacking certain phenotype and identify cells that have lost the function of certain genes and exhibit the specified phenotype. However, complementation screen can also be done in vitro in cell-free systems, either by testing each candidate individually or as pools of individuals.

In certain occasions, there is a need to recover a clone of a cell under conditions wherein a cell is selectable. That can mean selecting from a population of cells, a subpopulation or a single cell possessing a common given property such as the presence or absence of fluorescent markers, or the presence or absence of positive or negative selectable markers, and obtaining a clone of each selected cell. The cells can be selected under conditions that will completely or nearly completely eliminate any cell that does not have the desired property of the cells to be selected. For example, by growing cells in selective media, only cells possessing a certain desired property will survive. The surviving cells can be cloned using standard cell and molecular biology protocols (see Current Protocols in Cell Biology, CD-ROM Edition, ed. by Juan S. Bonifacnio, Jennifer Lippincott-Schwartz, Joe B. Harford, and Kenneth M. Yamada, John Wiley & Sons, 1999). Alternatively, cells possessing a desired property can be selected from a population based on the observation of a certain discernable phenotype, such as the presence or absence of fluorescent markers. The selected cells can then be cloned using standard cell and molecular biology protocols (see Current Protocols in Cell Biology, CD-ROM Edition, ed. by Juan S. Bonifacino, Jennifer Lippincott-Schwartz, Joe B. Harford, and Kenneth M. Yamada, John Wiley & Sons, 1999).

“Compound” in its broadest sense shall include (but is not limited to) chemical compounds (organic or inorganic), macromolecules such as polynucleotides, polypeptides, polysaccharides, lipids, or derivatives thereof.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

The terms “epitope” and “epitope tag”, as used herein, are meant to refer to any of various convenient molecular markers known in the art, such as hemaglutinin or FLAG, so that the level of a polypeptide can be confirmed in a Western blot using, for example, a suitable anti-flu or anti-FLAG antibody.

The term “equivalent” is understood to include polypeptides or nucleotide sequences that are functionally equivalent or possess an equivalent activity as compared to a given polypeptide or nucleotide sequence. Equivalent nucleotide sequences will include sequences that differ by one or more nucleotide substitutions, additions or deletions, such as allelic variants; and will, therefore, include sequences that differ from the nucleotide sequence of a particular gene, due to the degeneracy of the genetic code. Equivalent polypeptides will include polypeptides that differ by one or more amino acid substitutions, additions or deletions, which amino acid substitutions, additions or deletions leave the function and/or activity of the polypeptide substantially unaltered. A polypeptide equivalent to a given polypeptide could e.g. be the polypeptide that performs the same function in another species. For example, murine ubiquitin herein is considered an equivalent of human ubiquitin.

“G-protein” as used herein means heterotrimeric G proteins, including the α, β and γ subunits, as well as the Ras superfamily of small G-proteins, and fragments thereof. The Ras super family of small G proteins shall include, but are not limited to, Ras, Ran, Rho, Arf, Rab subfamily of small G-proteins. In addition, G-protein includes proteins that are structurally and/or functionally similar to the high eukaryotic G-proteins, but are found in other species, such as Ste4p, Ste18p of yeast, etc.

As used herein, the terms “gene”, “recombinant gene” and “gene construct” refer to a nucleic acid comprising an open reading frame encoding a polypeptide, including both exon and (optionally) intron sequences. The term “intron” refers to a DNA sequence present in a given gene which is not translated into protein and is generally found between exons.

A “recombinant gene” refers to nucleic acid encoding a polypeptide and comprising—encoding exon sequences, though it may optionally include intron sequences which are derived from, for example, a chromosomal gene or from an unrelated chromosomal gene.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity with another sequence.

The term “interact” as used herein is meant to include detectable interactions (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject polypeptides preferably includes no more than 10 kilobases (kb) of nucleic acid sequence which naturally immediately flanks the gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Kit” as used herein means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. The individual components of the kit may or may not be from the same supplier, or manufacturer. A component can either be purchased as a part of the kit, or generated by user “in-house” according to the instruction of the kit.

“Instruction(s)” as used herein means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can contain one or multiple documents or future updates. Instruction also includes patents, published patent applications, published scientific literatures or references, etc.

“Identify” or “identification” as used herein means selecting, screening, or finding at least one candidate possessing a desired property, from a pool of more than 2 candidates. The desired property can be, for example, a mutation or a compound that can cause a conformational change in a given protein, a condition or stimulus that can cause a conformational change in a given protein, etc. In certain preferred embodiments, identification may also include further characterization (see above) of the identified candidate.

“Library” as used herein generally means a multiplicity of member components constituting the library which member components individually differ with respect to at least one property, for example, a chemical compound library. Particularly, as will be apparent to skilled artisan, “library” means a plurality of nucleic acids/polynucleotides, preferrably in the form of vectors comprising functional elements (promoter, transcription factor binding sites, enhancer, etc.) necessary for expression of polypeptides, either in vitro or in vivo, which are functionally linked to coding sequences for polypeptides. The vector can be a plasmid or a viral-based vector suitable for expression in prokaryotes or eukaryotes or both, preferably for expression in mammalian cells. There should also be at least one, preferably multiple pairs of cloning sites for insertion of coding sequences into the library, and for subsequent recovery or cloning of those coding sequences. The cloning sites can be restriction endonuclease recognition sequences, or other recombination based recognition sequences such as loxP sequences for Cre recombinase, or the Gateway system (Life Technologies, Inc.) as described in U.S. Pat. No. 5,888,732, the contents of which is incorporated by reference herein. Coding sequences for polypeptides can be cDNA, genomic DNA fragments, or random/semi-random polynucleotides. The methods for cDNA or genomic DNA library construction are well-known in the art, which can be found in a number of commonly used laboratory molecular biology manuls (see below).

The term “modulation” as used herein refers to both upregulation (i.e., activation or stimulation, e.g., by agonizing or potentiating) and downregulation (i.e. inhibition or suppression e.g., by antagonizing, decreasing or inhibiting) of an activity.

The term “mutation” or “mutated” as it refers to a gene or nucleic acid means an allelic or modified form of a gene or nucleic acid, which exhibits a different nucleotide sequence and/or an altered physical or chemical property as compared to the wild-type gene or nucleic acid. Generally, the mutation could alter the regulatory sequence of a gene without affecting the polypeptide sequence encoded by the wildtype gene. But more commonly, a mutated gene or nucleic acid will either completely lose the ability to encode a polypeptide (null mutation) or encode a polypeptide with an altered property, including a polypeptide with reduced or enhanced biological activity, a polypeptide with novel biological activity, or a polypeptide that interferes with the function of the corresponding wild-type polypeptide. Alternatively, a mutation may take advantage of the degeneracy of the genetic code, by replacing a triplett codon by a different triplett codon that nevertheless encodes the same amino acid as the wild-type triplett codon. Such replacement may, for example, lead to increased stability of the gene or nucleic acid under certain conditions. Furthermore, a mutation may comprise a nucleotide change in a single position of the gene or nucleic acid, or in several positions, or deletions or additions of nucleotides in one or several positions.

The term “reduced-associating mutant” as used herein means a mutant polypeptide that exhibits reduced affinity for its normal binding partner. For example, a reduced-associating mutant of the ubiquitin N-terminus (also known as “Nᵤₓ”) is a polypeptide that exhibits reduced affinity for its normal binding partner—the C-terminal half of ubiquitin (C_{ub}), to the point that it will show reduced association or not associate with a wild-type C_{ub} and form a “quasi-wild-type ubiquitin” without the supplemented binding affinity between two polypeptides fused to Nᵤₓ and C_{ub}, respectively. In a preferred embodiment of the invention, such mutations in Nux are certain missense mutations introduced to either the 3^{rd} or the 13^{th} amino acid residue of the wild-type ubiquitin. Different missense mutations at these positions may differentially affect the affinity/association between Nᵤₓ and C_{ub}, thereby providing different sensitivity of the assay as disclosed by the instant invention. These missense point mutations can be routinely introduced into cloned genes using standard molecular biology protocols, such as site-directed mutagenesis using PCR.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant gene is present and/or expressed or disrupted in some tissues but not others.

As used herein, the term “nucleic acid,” in its broadest sense, refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense and double-stranded polynucleotides.

Specifically, “nucleic acid(s)” may refer to polynucleotides that contain information required for transcription and/or translation of polypeptides encoded by the polynucleotides. These include, but are not limited to, plasmids comprising transcription signals (e.g. transcription factor binding sites, promoters and/or enhancers) functionally linked to downstream coding sequences for polypeptides, genomic DNA fragments comprising transcription signals (e.g. transcription factor binding sites, promoters and/or enhancers) functionally linked to downstream coding sequences for polypeptides, cDNA fragments (linear or circular) comprising transcription signals (e.g. transcription factor binding sites, promoters and/or enhancers) functionally linked to downstream coding sequences for polypeptides, or RNA molecules comprising functional elements for translation either in vitro or in vivo or both, which are functionally linked to sequences encoding polypeptides. These polynecleotides should also be understood to include, as equivalents, analogs either RNA or DNA made from nucleotide analogs, and, as applicable to the embodiment being described, single (sense or antisense) and double-stranded polynucleotides. These polynucleotides can be in an isolated form, e.g. an isolated vector, or included into the episome or the genome of a cell.

The term “nucleotide sequence complementary to the nucleotide sequence set forth in SEQ ID NO. x” refers to the nucleotide sequence of the complementary strand of a nucleic acid strand having SEQ ID NO. x. The term “complementary strand” is used herein interchangeably with the term “complement”. The complement of a nucleic acid strand can be the complement of a coding strand or the complement of a non-coding strand.

“Nucleotide phosphate” referrs to any one or more of the following and derivatives or variants: AMP, ADP, ATP, TMP, TDP, TTP, CMP, CDP, CTP, GMP, GDP, GTP, cAMP, cTMP, cGMP, cCMP, dAMP, dADP, dATP, dTMP, dTDP, dTTP, dCMP, dCDP, dCTP, dGMP, dGDP, dGTP, ddAMP, ddADP, ddATP, ddTMP, ddTDP, ddTTP, ddCMP, ddCDP, ddCTP, ddGMP, ddGDP, ddGTP. In dNTP, either 3′- or 2′- can be —OH. Modifications (including replacement of O, N, or P atoms by S or others) on sugar rings, bases, and/or phosphate groups are all considered derivatives or variants.

As is well known, genes or a particular polypeptide may exist in single or multiple copies within the genome of an individual. Such duplicate genes may be identical or may have certain modifications, including nucleotide substitutions, additions or deletions, which all still code for polypeptides having substantially the same activity. Moreover, certain differences in nucleotide sequences may exist between individual organisms, which are called alleles. Such allelic differences may or may not result in differences in amino acid sequence of the encoded polypeptide yet still encode a polypeptide with the same biological activity.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co.; San Diego, Calif. USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

Preferred nucleic acids have a sequence at least 70%, and more preferably 80% identical and more preferably 90% and even more preferably at least 95% identical to an nucleic acid sequence encoding any one of the polypeptides of the instant application. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987) 25: 351-360. Another method, GAP, uses the alignment method of Needleman et al., J. Mol. Biol. (1970) 48: 443-453. GAP is best suited for global alignment of sequences. A third method, BestFit, functions by inserting gaps to maximize the number of matches using the local homology algorithm of Smith and Waterman, Adv. Appl. Math. (1981) 2: 482-489.

“Pharmaceutical composition” of the present invention comprise any one or more of the described compounds, or compositions of the present invention, or a pharmaceutically acceptable salt thereof, together with a pharmaceutically acceptable carrier in accordance with the properties and expected performance of such carriers which are well-known in the pertinent art.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNTA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a natural or recombinant gene product or fragment thereof which is not a nucleic acid.

A “protein of therapeutic, physiological or biological interest” shall mean a polypeptide for which exists at least one publicly available document, for example, without limitation, a published patent document or an article in the scientific literature, in which document a causal relationship is shown or proposed between said polypeptide and a state of a biological system, or a particular change of state of a biological system, which state or change of state may be desirable or undesirable. An example for a polypeptide with a proposed causal relationship to an undesirable state of a biological system is, without limitation, the ScPrP polypeptide, the proposed cause for Bovine Spongiforme Enzephalopathy (BSE). An example for a polypeptide with a proposed causal relationship to a desirable state of a biological system is, without limitation, the unmutated form of CFTR (Cystic Fibrosis Conductance Transmembrane Regulator).

The term “recombinant protein” refers to a polypeptide which is produced by recombinant DNA techniques, wherein generally, DNA encoding a polypeptide is inserted into a suitable expression vector which is in turn used to transform a host cell to produce the polypeptide encoded by said DNA. This polypeptide may be one that is naturally expressed by the host cell, or it may be heterologous to the host cell, or the host cell may have been engineered to have lost the capability to express the polypeptide which is otherwise expressed in wild type forms of the host cell. The polypeptide may also be a fusion polypeptide. Moreover, the phrase “derived from”, with respect to a recombinant gene, is meant to include within the meaning of “recombinant protein” those proteins having an amino acid sequence of a native polypeptide, or an amino acid sequence similar thereto which is generated by mutations, including substitutions, deletions and truncation, of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidonimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

“Reporter moiety” is a reporter for the cleavage of the peptide bond between C_{ub} and RM. In some cases, the UBP-cleaved RM is unstable due to the fact that the first amino acid is non-MET and RM will be degraded at the presence of a functional N-end rule system. Alternatively, RM may be stable if the first amino acid is Met or any other stabilizing amino acid residue. The detection of the RM activity can be through a variety of means. It can be detected by Western blot using an antibody against the RM or an attached epitope to reveal the cleaved RM and the uncleaved RM. It can be detected by degree of enzymatic activity of the RM is the RM is an enzyme with a non-Met first amino acid. It can be detected by the strength of the fluorescent signal of RM if the RM is a fluorescent protein with a non-Met first amino acid. For example, the reporter moiety may be chosen from the list of URA3, HIS3, LYS2, HygTlz, Tkneo, TkBSD, PACTk, HygCoda, Codaneo, CodaBSD, PACCoda, Tk, codA, and GPT2. The reporter moiety may also be TRP1, CYH2, CAN1, HPRT, beta-galactosidase or a luciferase. Futhermore, the reporter moiety may also be a fluorescent marker, e.g. GFP, YFP, BFP, or RFP, a transcription factor, e.g. hTBP1 (human TATA binding protein 1), or DHFR. A skilled artisan will be able to envisage other reporter moieties based on the description of the instant application and common knowledge in the art.

“Transcription” is a generic term used throughout the specification to refer to a process of synthesizing RNA molecules according to their corresponding DNA template sequences, which may include initiation signals, enhancers, and promoters that induce or control transcription of protein coding sequences with which they are operably linked. “Transcriptional repressor,” as used herein, refers to any of various polypeptides of prokayotic or eukaryotic origin, or which are synthetic artificial chimeric constructs, capable of repression either alone or in conjunction with other polypeptides and which repress transcription in either an active or a passive manner. It will also be understood that the transcription of a recombinant gene can be under the control of transcriptional regulatory sequences which are the same or which are different from those sequences which control transcription of the naturally-occurring forms of the recombinant gene, or its components.

“Translation” as used herein is a generic term used to describe the synthesis of protein or polypeptide on a template, such as messenger RNA (mRNA). It is the making of a protein/polypeptide sequence by translating the genetic code of an mRNA molecule associated with a ribosome. The whole process can be performed in vivo inside a cell using a protein translation machinery of the cell, or be performed in vitro using cell-free systems, such as reticulocyte lysates or any other equivalents. The RNA template for translation may be separately provided either directly as RNA or indirectly as the product of transcription from a provided DNA template, such as a plasmid.

“Translationally providing” means providing a polypeptide/protein by way of translation. As defined above, translation is a process that can be done in vivo inside a cell using protein translation machinery of the cell, or be performed in vitro using cell-free systems, such as reticulocyte lysates or any other equivalents. The RNA template for translation may be separately provided either directly as RNA or indirectly as the product of transcription from a provided DNA template, such as a plasmid. The template DNA can be introduced into a host/target cell by a variety of standard molecular biology procedures, such as transformation, transfection, mating or cell fusion, or can be provided to an in vitro translation reaction directly.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the polypeptide is disrupted.

As used herein, the term “transgene” means a nucleic acid sequence (encoding, e.g., one of the polypeptides, or an antisense transcript thereto) which has been introduced into a cell. A transgene could be partly or entirely heterologous, i.e., foreign, to the transgenic animal or cell into which it is introduced, or, homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the polypeptide, e.g. either agonistic or antagonistic forms. However, transgenic animals in which the recombinant—gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treating” as used herein is intended to encompass curing as well as ameliorating at least one symptom of the condition or disease. The term also means, the context of “treating . . . with a stimulus,” contacting, affecting, effecting, causing to happen, exposing to (an environmental alteration), etc.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “ubiquitin” as used herein refers to an abundant 76 amino acid residue polypeptide that is found in all eukaryotic cells. The ubiquitin polypeptide is characterized by a carboxy-terminal glycine residue that is activated by ATP to a high-energy thiol-ester intermediate in a reaction catalyzed by a ubiquitin-activating enzyme (E1). The activated ubiquitin is transferred to a substrate polypeptide via an isopeptide bond between the activated carboxy-terminus of ubiquitin and the epsilon-amino group of a lysine residue(s) in the protein substrate. This transfer requires the action of ubiquitin conjugating enzymes such as E2 and, in some instances, E3 activities. The ubiquitin modified substrate is thereby altered in biological function, and, in some instances, becomes a substrate for components of the ubiquitin-dependent proteolytic machinery which includes both UBP enzymes as well as proteolytic proteins which are subunits of the proteasome. As used herein, the term “ubiquitin” includes within its scope all known as well as unidentified eukaryotic ubiquitin homologs of vertebrate or invertebrate origin which call be classified as equivalents of human ubiquitin. Examples of ubiquitin polypeptides as referred to herein include the human ubiquitin polypeptide which is encoded by the human ubiquitin encoding nucleic acid sequence (GenBank Accession Nunbers: U49869, X04803). Equivalent ubiquitin polypeptide encoding nucleotide sequences are understood to include those sequences that differ by one or more nucleotide substitutions, additions or deletions, such as allelic variants as well as sequences which differ from the nucleotide sequence encoding the human ubiquitin coding sequence due to the degeneracy of the genetic code. Another example of a ubiquitin polypeptide as referred to herein is murine ubiquitin which is encoded by murine ubiquitin encoding nucleic acid sequence (GenBank Accession Number: X51730). It will be readily apparent to the person skilled in the art how to modify the methods and reagents provided by the present invention to the use of ubiquitin polypeptides other than human ubiquitin.

The term “ubiquitin-like protein” as used herein refers to a group of naturally occurring proteins, not otherwise describable as ubiquitin equivalent, but which nonetheless show strong amino acid homology to human ubiquitin. As used herein this term includes the polypeptides NEDD8, UBL1, NPVAC, and NPVOC. These “ubiquitin-like proteins” are at least over 40% identical in sequence to the human ubiquitin polypeptide and contain a pair of carboxy-terminal glycine residues which function in the activation and transfer of ubiquitin to target substrates as described supra.

As used herein, the term “ubiquitin-related protein” as used herein refers to a group of naturally occurring proteins, not otherwise describable as ubiquitin equivalents, but which nonetheless show some relatively low degree (<40% identity) of amino acid homology to human ubiquitin. These “ubiquitin-related” proteins include human Ubiquitin Cross-Reactive Protein (UCRP, 36% identical to huUb, Accession No. P05161), FUBI (36% identical to huUb, GenBank Accession No. AA449261), and Sentrin/Sumo/Pic1 (20% identical to huUb, GenBank Accession No. U83117). The term “ubiquitin-related protein” as used herein further pertains to polypeptides possessing a carboxy-terminal pair of glycine residues and which function as protein tags through activation of the carboxy-terminal glycine residue and subsequent transfer to a protein substrate.

The term “ubiquitin-homologous protein” as used herein refers to a group of naturally occurring proteins, not otherwise describable as ubiquitin equivalents or ubiquitin-like or ubiquitin-related proteins, which appear functionally distinct from ubiquitin in their ability to act as protein tags, but which nonetheless show some degree of homology to human ubiquitin (34-41% identity). These “ubiquitin-homologous proteins” include RAD23A (36% identical to huUb, SWISS-PROT. Accession No. P54725), RAD23B (34% identical to huUb, SWISS-PROT. Accession No. P54727), DSK2 (41% identical to huUb, GenBank Accession No. (L40587), and GDX (41% identical to huUB, GenBank Accession No. J03589). The term “ubiquitin-homologous protein” as used herein is further meant to signify a class of ubiquitin homologous polypeptides whose similarity to ubiquitin does not include glycine residues in the carboxy-terminal and penultimate residue positions. Said proteins appear functionally distinct from ubiquitin, as well as ubiquitin-like and ubiquitin-related polypeptides, in that, consistent with their lack of a conserved carboxy-terminal glycine for use in an activation reaction, they have not been demonstrated to serve as tags to other proteins by covalent linkage.

The term “ubiquitin conjugation machinery” as used herein refers to a group of proteins which function in the ATP-dependent activation and transfer of ubiquitin to substrate proteins. The term thus encompasses: E1 enzymes, which transform the carboxy-terminal glycine of ubiquitin into a high energy thiol intermediate by an ATP-dependent reaction; E2 enzymes (the UBC genes), which transform the E1-S˜Ubiquitin activated conjugate into an E2-S˜Ubiquitin intermediate which acts as a ubiquitin donor to a substrate, another ubiquitin moiety (in a poly-ubiquitination reaction), or an E3; and the E3 enzymes (or ubiquitin ligases) which facilitate the transfer of an activated ubiquitin molecule from an E2 to a substrate molecule or to another ubiquitin moiety as part of a polyubiquitin chain. The term “ubiquitin conjugation machinery”, as used herein, is further meant to include all known members of these groups as well as those members which have yet to be discovered or characterized but which are sufficiently related by homology to known ubiquitin conjugation enzymes so as to allow an individual skilled in the art to readily identify it as a member of this group. The term as used herein is meant to include novel ubiquitin activating enzymes which have yet to be discovered as well as those which function in the activation and conjugation of ubiquitin-like or ubiquitin-related polypeptides to their substrates and to poly-ubiquitin-like or poly-ubiquitin-related protein chains.

The term “ubiquitin-dependent proteolytic machinery” as used herein refers to proteolytic enzymes which function in the biochemical pathways of ubliquitin, ubiquitin-like, and ubiquitin-related proteins. Such proteolytic enzymes include the ubiquitin C-terminal hydrolases, which hydrolyze the linkage between the carboxy-terminal glycine residue of ubiquitin and various adducts; UBPs, which hydrolyze the glycine 76 lysine 48 linkage between cross-linked ubiquitin moieties in poly-ubiquitin conjugates; as well as other enzymes which function in the removal of ubiquitin conjugates from ubiquitinated substrates (generally termed “deubiquitinating enzymes”). The aforementioned protease activities function in the removal of ubiquitin units from a ubiquitinated substrate following or during uibiquitin-dependent degradation as well as in certain proofreading functions in which free ubiquitin polypeptides are removed from incorrectly ubiquitinated proteins. The term “ubiquitin-dependent proteolytic machinery” as used herein is also meant to encompass the proteolytic subunits of the proteasome (including human proteasome subunits C2, C3, C5, C8, and C9). The term “ubiquitin-dependent proteolytic machinery” as used herein thus encompasses two classes of proteases: the deubiquitinating enzymes and the proteasome subunits. The protease functions of the proteasome subunits are not known to occur outside the context of the assembled proteasome, however independent functioning of these polypeptides has not been excluded.

The term “ubiquitin system” as referred to herein is meant to describe all of the aforementioned components of the ubiquitin biochemical pathways including ubiquitin, ubiquitin-like proteins, ubiquitin-related proteins, ubiquitin-homologous proteins, ubiquitin conjugation machinery, ubiquitin-dependent proteolytic machinery, or any of the substrates which these ubiquitin system components act upon.

3. Selectable Reporters for Yeast and Mammalian Cells

The invention provides negative selectable marker genes or “negative selectable reporter moieties” which can be used in a eulcaryotic host cell, preferably a yeast or a mammalian cell, and which can be selected against under appropriate conditions. In preferred embodiments, the selectable reporter is provided as a fusion polypeptide with a carboxy- or C-terminal subdomain of ubiquitin (or Cub) and is in some embodiments of the present invention altered so as to encode a non-methionine amino acid residue at the junction with the Cub. The non-methionine amino acid residue is preferably an amino acid which is recognized by the N-end rule ubiquitin protease system (e.g. an arginine, lysine, histidine, phenylalanine, tryptophan, tyrosine, leucine or isoleucine residue) and which, when present at the amino-terminal of the negative selectable marker, targets the negative selectable marker for rapid proteolytic degradation. It will be readily apparent to the person skilled in the art that the choice of amino acid residue recognized by the N-end rule ubiquitin protease system that is optimal for a given host cell depends on the type of host cell used, as, for example, the ubiquitin-dependent proteolytic machinery in yeast cells recognizes a slightly different set of amino acid residues than the ubiquitin-dependent proteolytic machinery in mammalian cells (Varshavsky (1992) Cell 69: 725-35).

A preferred example of a negative selectable marker gene for use in yeast is the URA3 gene which can be both selected for (positive selection) by growing ura3 auxotroplic yeast strains in the absence of uracil, and selected against (negatively selection) by growing cells on media containing 5-fluoroorotic acid (5-FOA) (see Boeke, et al. (1987) Methods Enzymol 154: 164-75). The concentration of 5-FOA can be optimized by titration so as to maximally select for cells in which the URA3 reporter is inactivated by proteolytic degradation to some preferred extent. For example, relatively high concentrations of 5-FOA can be used which allow only cells expressing very low steady-state levels of URA3 reporter to survive. Such cells will correspond to those in which the first and second ubiquitin subdomain fusion proteins have a relatively high affinity for one another, resulting in efficient reassembly of the N_{ub} and C_{ub} fragments and a correspondingly efficient release of the n-URA3 labilized marker. In contrast, lower concentrations of 5-FOA can be used to select for protein binding partners with relatively weak affinities for one another. In addition, proline can be used in the media as a nitrogen source to make the cells hypersensitive to the toxic affects of tile 5-FOA (McCusker & Davis (1991) Yeast 7: 607-8). Accordingly, proline concentrations, as well as 5-FOA concentrations can be titrated so as to obtain an optimal selection for URA3 reporter deficient cells. Therefore the use of URA3 as a negative selectable marker allows a broad range of selective stringencies which can be adapted to minimize false positive background noise and/or to optimize selection for high affinity binding interactions. Other negative selectable markers which operate in yeast and which can be adapted to the method of the invention are included within the scope of the invention.

Another example of a negative selectable marker gene for use in yeast in the TRP1 gene which can be both selected for (positive selection) by growing trp1 auxotrophic yeast strains in the absence of tryptophan, and selected against (negatively selection) by growing cells on media containing 5-fluoroanthranilic acid (5-FAA) (Toyn et al. (2000) Yeast 16: 553-560).

Two other negative selectable marker genes for the use in yeast are CYH2 and CAN1 both of which can be selected against (negative selection) by growing cells on media containing cycloheximide or canavanine (The yeast two-hybrid system, ed. by Bartel and Fields, Oxford University Press: 1997).

Numerous selectable markers which operate in mammalian cells are known in the art and can be adapted to the method of the invention so as to allow direct negative selection of interacting proteins in mammalian cells. Examples of mammalian negative selectable markers include Thymidine kinase (Tk) (Wigler et al. (1977) Cell 11: 223-32; Borrelli et al. (1988) Proc. Natl. Acad. Sci. USA 85: 7572-76) of the Herpes Simplex virus, the human gene for hypoxanthine phosphoriboxyl transferase (HPRT) (Lester et al. (1980) Somatic Cell Genet. 6: 241-59; Albertini et al. (1985) Nature 316: 369-71) and Cytidine deaminase (codA) from E. coli (Mullen et al. (1992) Proc. Natl. Acad. Sci. USA 89: 33-37; Wei and Huber (1996) J. Biol. Chem. 271: 3812-16). For example: the Tk gene can be selected against using Gancyclovir (GANC) (e.g. using a 1 uM concentration) and codA gene can be selected against using 5-Fluor Cytidin (5-FIC) (e.g. using a 0.1-1.0 mg/ml concentration). In addition, certain chimeric selectable markers have been reported (Karreman (1998) Gene 218: 57-61) in which a functional mammalian negative selectable marker is fused to a functional mammalian positive selectable marker such as Hygromycinresistance (Hyg^{R}, neomycin resistance (neo^{R}), puromycin resistance (PAC^{R}) or Blasticidin S resistance (BlaS^{R}). These produce various Tk-based positive/negative selectable markers for mammalian cells such as HygTk, Thneo, TkBSD, and PACTk, as well as various codA-based positive/negative selectable markers for mammalian cells such as HygCoda, Codaneo, CodaBSD, and PACCoda. Tk-neo reporters which incorporate luciferase, green fluorescent protein and/or beta-galactosidase have also been recently reported (Strathdee et al. (2000) BioTechniques 28: 210-14). These vectors have the advantage of allowing ready screening of the “positive” marker/reporter by fluorescent and/or immunofluorescent microscopy. The use of such positive/negative selectable markers affords the advantages mentioned above for URA3 as a reporter in yeast, inasmuch as they allow mammalian cells to be assessed by both positive and negative selection methods for the expression and relative steady-state level of the reporter fusion. For example, Rojo-Niersbach et al reported the use of GPT2 (Guanine Phosphoryl Transferase 2) in mammalian cells as a basis for the selection of protein interactions (Biochem. J. 348: 585-590, 2000).

In certain embodiments, the invention further provides positive selectable marker genes or “positive selectable reporter moieties” which can be used in a eukaryotic host cell, preferably a yeast or a mammalian cell, and which can be selected for under appropriate conditions. In preferred embodiments, the selectable reporter is provided as a fusion polypeptide with a carboxy- or C-terminal subdomain of ubiquitin (or C_{ub}) and is in some embodiments of the present invention altered so as to encode a non-methionine amino acid residue at the junction with the C_{ub} as further described supra. In principle, any non-redundant gene in a synthetic pathway that is essential to the survival of the cell can be used for the construction of an auxotrophic positive selectable marker, but frequently used such makers include, without limitation, HIS3, LYS2, LEU2, TRP2, ADE2. Usually, a cell line is constructed that is deficient in the marker gene, and that can only grow on media supplemented with the corresponding metabolic product, i.e. histidine, lysine, leucine, tryptophane or adenine. When used for selection, a desirable phenotype, i.e. expression of a desired recombinant gene, is linked to the expression of the gene the cell is deficient in by transforming cells with gene constructs comprising both the desired recombinant gene and a recombinant form of the marker gene. Other positive selectable markers include antibiotic resistance markers, e.g. Hygromycinresistance (Hyg^{R}), neomycin resistance (neo^{R}), puromycin resistance (PAC^{R}) or Blasticidin S resistance (BlaS^{R}), as mentioned supra, or any other antibiotic resistance marker. Here, expression of a desired recombinant gene is linked to the expression of the antibiotic resistance marker by transforming cells with gene constructs comprising both the desired recombinant gene and a recombinant form of the antibiotic resistance marker gene. Selection is then carried on media containing the antibiotic, e.g. Hygromycin, neomycin, puromycin or Blasticidin S. Furthermore, the above mentioned combinations of positive and negative markers can also be employed.

Other advantages of these mammalian reporter and selectable marker constructs will be apparent to the skilled artisan.

4. Components of N-end Rule Proteolytic Pathway

“N-end rule” system for proteolytic degradation is a particular branch of the ubiquitin-mediated proteolytic pathway present in eulcaryotic cells (Bachmair et al., Science 234: 179-86, 1986). This system operates to degrade a cellular polypeptide at a rate dependent upon the amino-terminal amino acid residue of that polypeptide. Protein translation ordinarily initiates with an ATG methionine codon and so most polypeptides have an amino-terminal methionine residue and are typically relatively stable in vivo. For example, in the yeast S. cerevisiae, a beta-galactosidase polypeptide with a methionine amino terminus has a half-life of >20 hours (Varshavsky, Cell 69: 725-35, 1992). Under certain circumstances, however, polypeptides possessing a non-methionine amino-terminal residue can be created. For example, when an endoprotease hydrolyzes and thus cleaves a unique polypeptide bond (Y-n) internal to a polypeptide, it results in the release of two separate polypeptides—one of which possesses-an amino-terminal amino acid, n, which may not be methionine. For example, the endoproteases, UBP, ubiquitin specific proteases, which are a preferred component of the present invention, will cleave a polypeptide bond carboxy-terminal to the final glycine residue (codon 76) of ubiquitin, regardless of what the next codon is. In the normal function of the cell, these UBPs serve to cleave a polyubiquitin precursor or other ubiquitin fusion proteins to liberate individual ubiquitin units. However it can also be used to generate a target polypeptide with virtually any amino-terminal residue by merely fusing the target polypeptide in-frame to a codon corresponding to the desired amino-terminal amino acid (n), which codon, in turn, is fused downstream of ubiquitin (typically contiguous with ubiquitin Gly codon 76). The resulting target gene chimera construct, has the general structure Ubiquitin-n-target. Preferred target constructs further comprise an epitope tag (Ep) so that the resulting target gene chimera construct has the general structure Ubiquitin-n-Ep-target, which results in the eventual production of a polypeptide of the general structure n-Ep-target. Constitutively active ubiquitin-specific protease activities present in eukaryotic cells will result in the endoproteolytic processing of the Ubiquitin-n-target polypeptide into Ubiquitin and n-target entities. The n-target polypeptide is further acted upon by the components of the N-end rule system as described below. If the target polypeptide is a negative selection marker (NSM) and if n is an amino acid residue (such as arg) which potentiates rapid degradation by the N-end rule system, then cells expressing intact Ubiquitin-n-NSM can be selected against while cells in which the fusion is clipped into a relatively labile n-NSM polypeptide can be selected for.

It has been determined, with reasonable reliability, the relative effect of a given amino-terminal residue, n, upon target polypeptide stability. For example, when all 20 possible amino-terminal amino acid residues were tested to determine their effect on the stability of beta-galactosidase (utilizing a ubiquitin-n-beta-galactosidase chimeric fusion) in Saccharornyces cerevisiae, drastic differences were discovered (see Varshavsky, Cell 69: 725-35, 1992). For example when n was met, cys, ala, ser, thr, gly, val, or pro, the resulting polypeptide was very stable (half-life of >20 hours). When n was tyr, ile, glu, or gln, the resulting polypeptide possessed moderate protein stability (half-life of 10-30 minutes). In contrast, the residues arg, lys, phe, leu, trp, his, asp, and asn, all conferred low stability on the beta-galactosidase polypeptide (half-life of <3 minutes). The residue arginine (arg), when located at the amino terminus of a polypeptide, appears to generally confer the lowest stability. Thus, chimeric constructs and corresponding chimeric polypeptides employing an arg residue at the position n, described above, are generally preferred embodiments of the present invention. This is because a general goal of the invention is to eliminate the function of the target gene polypeptide in the cell.

The above described experiments establishing the relative half-lives conferred by each of the 20 possible amino terminal residues form the basis of the N-end rule. The N-end rule system components are those gene products which act to bring about the rapid proteolysis of polypeptides possessing amino-terminal residues which confer instability. The N-end rule system for proteolysis in eukaryotes appears to be a part of the general ubiquitin-dependent proteolytic system pathways possessed by apparently all eukaryotic cells. Briefly, this system involves the covalent tagging of a target polypeptide on one or more lysine residues by a ubiquitin polypeptide marker (to form a target(lys)-epsilon amino-gly(76)Ubiquitin covalent bond). Additional ubiquitin moieties may be subsequently conjugated to the target polypeptide and the resulting “ubiquitinated” target polypeptide is then subject to complete proteolytic destruction by a large (23S) multiprotein complex known as the proteasome. The enzymes which conjugate the ubiquitin moieties to the targeted protein include E2 and E3 (or ubiquitin ligase) functions. The E2 and E3 enzymes are thought to possess most of the specificity for ubiquitin dependent proteolytic processes.

A key component of the N-end rule proteolytic pathway in yeast is UBR1 (Bartel et al., EMBO J. 9: 3179-89, 1990), a gene which encodes an E3 like function which appears to recognize polypeptides possessing susceptible amino terminal residues and thereby facilitates ubiquitination of such polypeptides (Dohmen et al., Proc. Natl. Acad. Sci. USA 88: 7351-55, 1991). Accordingly UBR1 can be used as a regulatable N-end rule component which is the effector of proteolytic degradation of the target gene polypeptide. The UBR1 gene has now been cloned from a mammalian organism (Kwon et al., Proc. Natl. Acad. Sci. USA 95: 7893-903, 1998) as well as from yeast. Thus the construction of a UBR1 mouse cell line knockout is imminent and so control of the instability of n-RMs can be further manipulated by controlling the level of UBR1 expressed.

The UBR1 gene is particularly central to the invention because it can be selectively used in conjunction with any of the above described non-methionine “n” amino-terminal destabilizing residues including: the most destabilizing—arg; strongly destabilizing residues—such as lys phe, leu, tip, his, asp, and asn; and moderately destabilizing residues—such as tyr, ile, glu, or gln. Indeed, it is an object of the present invention to provide a means, where desired, to not completely shut-off a negative selectable marker's function, but merely to attenuate it to some set degree. This can be achieved using the method of the present invention in any of a number of ways. For example, a moderately destabilizing amino-terminal residue (n=tyr, ile, glu, or gln) can be deployed on the target polypeptide reporter—resulting in a less rapid removal of the target polypeptide pool.

Other N-end rule components for use in the present invention include S. cerevisiae UBC2 (RAD6), which encodes an E2 ubiquitin conjugating function which cooperates with the UBR1—encoded N-end rule E3 to promote multiubiquitination and subsequent degradation of N-end rule substrates (Dohmen et al., Proc. Natl. Acad. Sci. USA 88: 7351-55, 1991). Thus N-end rule directed proteolysis will not occur in the absence of either UBR1 or UBC2. This allows either gene to be used as the inducible “effector of targeted proteolysis” by the method of the present invention. Indeed, a target gene polypeptide possessing an N-end rule destabilizing amino-terminal amino acid (such as arg) will be stable until expression of either the UBR1 (E3) or the UBC2 (E2) is induced from the cognate inducible promoter construct.

Both UBR1 and UBC2 can be used in conjunction with any of the above described “n” amino-terminal destabilizing residues including: the most destabilizing—arg; strongly destabilizing residues—such as lys phe, leu, trp, his, asp, and asn; and moderately destabilizing residues—such as tyr, ile, glu, or gln. Still other alternative embodiments of the N-end rule component of the present invention are components of the N-end rule system which affect only a subset of the destabilizing residues. For example, the NTA1 deamidase (Baker and Varshavsky, J Biol Chem 270: 12065-74, 1995) functions to deaminate amino-terminal asn or gln residues (to form polypeptides with asp or glu amino-terminal residues respectively). Yeast strains harboring nta1 null alleles are unable to degrade N-end rule substrates that bear amino-terminal asn or gln residues. Thus, the NTA1 gene is an alternative embodiment of the N-end rule component of the present invention, but is used preferably in conjunction with a target gene polypeptide (n-target), in which n is either asn or gln. Similarly the ATE1 transferase (Balzi et al., J. Biol Chem 265: 7464-71, 1990) is an enzyme which acts to transfer the arg moiety from a tRNA˜Arg activated tRNA to amino-terminal glu or asp bearing polypeptides. The resulting arg-glu-polypeptide and arg-asp-polypeptide products are then susceptible to the E2/E3—mediated N-end rule dependent proteolytic processes described above. Thus, the ATE1 transferase is an alternative embodiment of the N-end rule component of the present invention, but its use is preferably tied to target gene polypeptides (n-target), in which n is asp, asn, or gln. Polypeptides bearing the latter two amino-terminal residues are first converted to polypeptides bearing one of the former two amino-terminal residues by NTA1 deamidase function described above.

From the description above, it is apparent to a skilled artisan that different cell types might possess different N-end rule components. Therefore, it might be necessary and important to genetically engineer a given cell line so that a complementation screen based on the instant invention can be successfully carried out in that given cell line. For example, many libraries or constructs generated for use in mammalian systems might be easily adapted for use in a different cell type if that cell type has the same or very similar N-end rule components and operates essentially the same as mammalian cells. However, if that cell type has dramatically different N-end rule components, it might be worthwhile to genetically modify the cell type so that available reagents can be readily used, rather than regenerate reagents for use in that particular cell line. For example, the N-end rule components may be provided as a clone so that it they can be put under the control of an inducible promoter (using standard subcloning methods well known in the art). It is also possible that other genetic engineering steps can be performed in a given cell type to make it suitable for expression of source DNA in libraries using mammalian expression vectors.

The techniques used for such genetic engineering involve stable expression of genes, which genes may potentially be heterologous to the cell type employed, and/or “knocking-out” genes, techniques which are well known in the art and can be readily appreciated by a skilled artisan.

It is also important to note here that, as is the case for the repressor of the present invention which is made subject to induction by an inducible promoter of the present invention, the N-end rule component must be available as a clone so that it can be put under the control of an inducible promoter (using standard subcloning methods known in the art). This can be achieved by first introducing genetically engineered copies of the inducible repressor and the inducible N-end rule component constructs, and subsequently deleting the normal chromosomal copies of these genes from the host by “knockout” methods. Such methods, we note here are well developed in the art—particularly n the case of both the yeast Saccharomyces cerevisiae and the mammal mouse. More convenient, however, is the availability of “knock-in” technology which allows the existing chromosomal copy of the gene to be modified to so that its native promoter is deleted and an inducible promoter is inserted in a single step. FIG. 2A diagrams this process for the replacement of the native promoter of the target gene with a repressible promoter, but this principle is also applicable to the replacement of the native promoter of the effector of suppression (i.e. the transcriptional repressor and/or the N-end rule component) with a suitable inducible promoter.

5. Ubiquitin Polypeptide Sequences

A complete and detailed description of certain C_{ub} and N_{ub} constructs which can be used in the method of the present invention have been described in U.S. Pat. Nos. 5,503,977 and 5,585,245. A background to the molecular biology of the ubiquitin proteolytic system in general, and the N-end rule system and ubiquitin sensor association assay is presumed of the skilled artisan seeking to practice the present invention. Briefly, ubiquitin (Ub) is a 76-residue, single-domain protein whose covalent coupling to other proteins yields branched Ub-protein conjugates and plays a role in a number of cellular processes, primarily through routes that involve protein degradation. Unlike the branched Ub conjugates, which are formed posttranslationally, linear Ub adducts are the translational products of natural or engineered Ub fusions. It has been shown that, in eulcaryotes, newly formed Ub fusions are rapidly cleaved at the Ub-polypeptide junction by Ub-specific proteases (UBPs). In the yeast Saccharomyces cerevisiae, there are at least five species of UBP. Recent work has shown that the cleavage of a Ub fusion by UBPs requires the folded conformation of Ub, because little or no cleavage is observed with fusions whose Ub moiety was conformationally destabilized by single-residue replacements or a deletion distant from the site of cleavage by UBPs.

The present invention relies in part upon the previously described split ubiquitin protein sensor system (see U.S. Pat. Nos. 5,503,977 & 5,585,245). Briefly, it has been demonstrated that an N-terminal ubiquitin subdomain and a C-terminal ubiquitin subdomain, the latter bearing a reporter extension at its C-terminus, when coexpressed in the same cell by recombinant DNA techniques as distinct entities, have the ability to associate, reconstituting a ubiquitin molecule which is recognized, and cleaved, by ubiquitin-specific processing proteases which are present in all eulcaryotic cells. This reconstituted ubiquitin molecule, which is recognized by ubiquitin-specific proteases, is referred to herein as a quasi-native ubiquitin moiety. As disclosed herein, ubiquitin-specific proteases recognize the folded conformation of ubiquitin. Remarkably, ubiquitin-specific proteases retained their cleavage activity and specificity of recognition of the ubiquitin moiety that had been reconstituted from two unlinked ubiquitin subdomains.

Ubiquitin is a 76-residue, single-domain protein comprising two subdomains which are relevant to the present invention the N-terminal subdomain and the C-terminal subdomain. The ubiquitin protein has been studied extensively and the DNA sequence encoding ubiquitin has been published (Ozkaynak et al., EMBO J. 6: 1429, 1987). The N-terminal subdomain (N_{ub}), as referred to herein, is that portion of the native ubiquitin molecule which folds into the only alpha-helix of ubiquitin interacting with two beta-strands. Generally speaking, this subdomain comprises amino acid residues from about residue number 1 to about residue number 34-37.

The C-terminal subdomain of ubiquitin (C_{ub}), as referred to herein, is that portion of the ubiquitin which is not a portion of the N-terminal subdomain defined in the preceding paragraph. Generally speaking, this subdomain comprises amino acid residues from about 35-38 to about 76. It should be recognized that by using only routine experimentation it will be possible to define with precision the minimum requirements at both ends of the N-terminal subdomain and the C-terminal subdomain which are necessary to be useful in connection with the present invention.

It is important to note that the terms N_{ub} refer, in preferred embodiments of the invention, to ubiquitin subdomain units which have been mutated so as to decrease their binding affinity, thereby making the C_{ub}/N_{ub} association dependent upon the binding of a second protein pair fused to the C_{ub} and N_{ub} subunits or a conformational change of a protein fused in between N_{ub} and C_{ub}. Suitable forms of N_{ub} are described below and still others are readily available to the skilled artisan by routine mutation and screening methods.

In order to study the interaction between members of a specific-binding pair, one member of the pair is fused to the N-terminal subdomain of ubiquitin and the other member of the specific-binding pair is fused to the C-terminal subdomain of ubiquitin. Since the members of the specific-binding pair (linked to subdomains of ubiquitin) have an affinity for one another, this affinity increases the “effective” (local) concentration of the N-terminal and C-terminal subdomains of ubiquitin, thereby promoting the reconstitution of a quasi-native ubiquitin moiety. For convenience, the term “quasi-native ubiquitin moiety” will be used herein to denote a moiety recognizable as a substrate by ubiquitin-specific proteases. In light of the fact that the N-terminal and C-terminal subdomains of ubiquitin associate to form a quasi-native ubiquitin moiety even in the absence of fusion of the two subdomains to individual members of a specific-binding pair, a further requirement is imposed in the present invention in order to increase the resolving capacity of the method for studying such interactions. The further requirement is that either the N-terminal subdomain of ubiquitin, or the C-terminal subdomain of ubiquitin, or both, must be mutationally altered to reduce their ability to produce, through their association, a quasi-native ubiquitin moiety. It will be recognized by one of skill in the art that the binding interaction studies described herein are carried out under conditions appropriate for protein/protein interaction. Such conditions are provided in vivo (i.e., under physiological conditions inside living cells) or in vitro, when parameters such as temperature, pH and salt concentration are controlled in a manner intended to mimic physiological conditions. The present invention preferably uses the disclosed in vivo screening methods which have the advantage of being subject to a powerful negative selection method.

The mutational alteration of a ubiquitin subdomain for use with the instant invention is preferably a point mutation. In light of the fact that it is essential that the reconstituted ubiquitin moiety must “look and feel” like native ubiquitin to a ubiquitin-specific protease, mutational alterations which would be expected to grossly affect the structure of the subdomain bearing the mutation are to be avoided. A number of ubiquitin-specific proteases have been reported, and the nucleic acid sequences encoding such proteases are also known (see e.g., Tobias et al., J. Biol. Chem. 266: 12021, 1991; Baker et al., J. Biol. Chem. 267: 23364, 1992). It should be added that all of the at least five ubiquitin-specific proteases in the yeast S. cerevisiae require a folded conformation of ubiquitin for its recognition as a substrate. Extensive deletions within the N- or C-terminal subdomains of ubiquitin are an example of the type of mutational alteration which would be expected to grossly affect subdomain structure and, therefore, are examples of types of mutational alterations which should be avoided.

In light of this consideration, the preferred mutational alteration within the N_{ub} subunit is a mutation in which an amino acid substitution is effected. For example, the substitution of an amino acid having chemical properties similar to the substituted amino acid (e.g., a conservative substitution) is preferred. Specifically, the desired mild perturbation of ubiquitin subdomain interaction is achieved by substituting a chemically similar amino acid residue which differs primarily in the size of its side chain. Such a steric perturbation is expected to introduce a desired (mild) conformational destabilization of a ubiquitin subdomain. The goal is to reduce the affinity of the N-terminal and C-terminal subdomains for one another, not necessarily to eliminate this affinity.

For example, the mutational alteration may be introduced into the N-terminal subdomain of ubiquitin. More specifically, a first neutral amino acid residue may be replaced with a second neutral amino acid having a side chain which differs in size from the first neutral amino acid residue side chain to achieve the desired decrease in affinity. For example, the first neutral amino acid residue isoleucine (either residue 3 or 13 of wild-type ubiquitin) may be replaced with a neutral amino acids which has a side chain which differs in size from isoleucine such as glycine, alanine or valine.

A wide variety of fusion construct combinations can be used in the methods of this invention. One strict requirement which applies to all N- and C-terminal fusion construct combinations is that the C-terminal subdomain must bear an amino acid (e.g., peptide, polypeptide or protein) extension. This requirement is based on the fact that the detection of interaction between two proteins of interest linked to two subdomains of ubiquitin is achieved through cleavage after the C-terminal residue of the quasi-native ubiquitin moiety, with the formation of a free reporter moiety (or peptide) that had previously been linked to a C-terminal subdomain of ubiquitin. Ubiquitin-specific proteases cleave a linear ubiquitin fusion between the C-terminal residue of ubiquitin and the N-terminal residue of the ubiquitin fusion partner, but they do not cleave an otherwise identical fusion whose ubiquitin moiety is conformationally perturbed. In particular, they do not recognize as a substrate a C-terminal subdomain of ubiquitin linked to a “downstream” reporter sequence, unless this C-terminal subdomain associates with an N-terminal subdomain of ubiquitin to yield a quasi-native ubiquitin moiety.

Furthermore, the characteristics of the C-terminal amino acid extension of the C-terminal ubiquitin subdomain must be such that the products of the cleaved fusion protein are distinguishable from the uncleaved fusion protein. In practice, this is generally accomplished by monitoring a physical property or activity of the C-terminal extension which is cleaved free from the C-terminal ubiquitin moiety. It is generally a property of the free C-terminal extension that is monitored as an indication that a quasi-native ubiquitin has formed, because monitoring of the quasi-native ubiquitin moiety directly is difficult in eukaryotic cells due to the presence of native ubiquitin. While unnecessary for the practice of the present invention, it would of course be appropriate to monitor directly the presence of the quasi-native ubiquitin as well, provided that this monitoring could be carried out in the absence of interference from native ubiquitin (for example, in prokaryotic cells, which naturally lack ubiquitin.

The size of the C-terminal extension which is released following cleavage of the quasi-native ubiquitin moiety within a reporter fusion by a ubiquitin-specific protease is a particularly convenient characteristic in light of the fact that it is relatively easy to monitor changes in size using, for example, electrophoretic methods. For instance, if the C-terminal reporter extension has a molecular weight of about 20 kD, the cleavage products will be distinguishable from the non-cleaved quasi-native ubiquitin moiety by virtue of the appearance of a previously absent reporter-specific 20 kD band following cleavage of the reporter fusion.

In light of the fact that the cleavage can take place, for example, in crude cell extracts or in vivo, it is generally not possible to monitor such changes in molecular weight of cleavage products by simply staining an electrophoretogram with a dye that stains proteins nonspecifically, because there are too many proteins in the mixture to analyze in this manner. One preferred method of analysis is immunoblotting. This is a conventional analytical method wherein the cleavage products are separated electrophoretically, generally in a polyacrylamide gel matrix, and subsequently transferred to a charged solid support (e.g., nitrocellulose or a charged nylon membrane). An antibody which binds to the reporter of the ubiquitin-specific protease cleavage products is then employed to detect the transferred cleavage products using routine methods for detection of the bound antibody.

Another useful method is immunoprecipitation of either a reporter-containing fusion to C-terminal subdomains of ubiquitin or the free reporter (liberated through the cleavage by ubiquitin-specific proteases upon reconstitution of a quasi-native ubiquitin moiety) with an antibody to the reporter. The proteins to be immunoprecipitated are first labeled in vivo with a radioactive amino acid such as ³⁵S-methionine, using methods routine in the art. A cell extract is then prepared, and reporter-containing proteins are precipitated from the extract using an anti-reporter antibody. The immunoprecipitated proteins are fractionated by electrophoresis in a polyacrylamide gel, followed by detection of radioactive protein species by autoradiography or fluorography.

A preferred experimental design is to extend the C-terminal subdomain of ubiquitin with a peptide containing an epitope foreign to the system in which the assay is being carried out. It is also preferable to design the experiment so that the C-terminal reporter extension of the C-terminal subdomain of ubiquitin is sufficiently large, i.e., easily detectable by the electrophoretic system employed. In this preferred embodiment, the C-terminal reporter extension of the C-terminal subdomain should be viewed as a molecular weight marker. The characteristics of the extension other than its molecular weight and immunological reactivity are not of particular significance. It will be recognized, therefore, that this C-terminal extension can represent an amalgam comprising virtually any amino acid sequence combination fused to an epitope for which a specifically binding antibody is available. For example, the C-terminal extension of the C-terminal ubiquitin subdomain may be a combination of the “ha” epitope fused to mouse DHFR (an antibody to the “HA” epitope is readily available).

Aside from the molecular weight of the C-terminal amino acid extension of the C-terminal ubiquitin subdomain, other characteristics can also be monitored in order to detect cleavage of a quasi-native ubiquitin moiety. For example, the enzymatic activity of some proteins can be abolished by extending their N-termini. Such a “reporter” enzyme, which, in its native form, exhibits an enzymatic activity that is abolished when the enzyme is N-terminally extended, can also serve as the C-terminal reporter linked to the C-terminal ubiquitin subdomain.

In this detection scheme, when the reporter is present as a fusion to the C-terminal ubiquitin subdomain, the reporter moiety is inactive. However, if the C-terminal ubiquitin subdomain and the N-terminal ubiquitin subdomain associate to reconstitute a quasi-native ubiquitin moiety in the presence of a ubiquitin-specific protease, the reporter moiety will be released, with the concomitant restoration of its enzymatic activity.

In preferred embodiments, the reporter moiety is a eukaryotic negative selectable marker (NSM) which has been engineered to be processed and released as an N-end rule-labile n-NSM fusion following UBP cleavage. The negative selectable markers (NSMs) for use in the invention are described elsewhere. The advantage of using an n-NSM fusion is that interaction of the specific binding pair can be directly selected for (as opposed to screened for) by virtue of the fact that only cells in which n-NSM has been released will survive negative selection.

The target gene reporter (negative selectable marker) must be fused downstream of a codon which encodes an N-end rule susceptible residue (n, as described above) and this residue, in term, must be fused in-frame to the carboxy-terminus of a ubiquitin coding sequence (generally the carboxy-terminus of a C-terminal ubiquitin subdomain (C_{ub}) which corresponds to gly76 of intact ubiquitin). The reason for constructing this extensive chimeric gene construct is to take advantage of the ability of constitutive ubiquitin-specific proteases to cleave any peptide bond which is carboxy-terminal to gly76 of an intact ubiquitin unit.

These UBPs normally functions to process poly-ubiquitin chains (the translational product of the tandem ubiquitin encoding sequences of eukaryotic genomes) into discrete (normally 76 a.a.) ubiquitin moieties which are used in ubiquitin-system pathways. In the method of the present invention, the UBPs serve as a convenient means to generate target gene polypeptides bearing specific amino-terminal residues (n). Nonetheless, it is understood that other alternatives to mammalian or yeast ubiquitin exist which can function in the method of the present invention. Such ubiquitin equivalents include, for example, ubiquitin mutants, ubiquitin-like proteins, ubiquitin-related proteins, and ubiquitin-homologous proteins. For example, ubiquitin-like proteins such as NEDD8, UBL1, FUBI, and UCRP, as well as analogous ubiquitin-related proteins such as SUMO/Sentrin/Pic1 may be used as ubiquitin equivalents in the method of the invention. Other proteins related to ubiquitin, but which are somewhat less homologous to it, include ubiquitin-homologous proteins such as Rad23 and Dsk2 whose similarity to ubiquitin does not include the presence of a carboxyl-terminal pair of glycines. These ubiquitin-like proteins share the common features of being related to ubiquitin by amino acid sequence homology and, with the apparent exception of the ubiquitin homologous proteins, of being covalently transferred to cellular protein targets post-translationally.

Indeed, the intended scope of the immediate invention encompasses any means known in the art by which a target polypeptide bearing an N-end rule susceptible residue (n=arg, lys, his, leu, phe, try, ile, trp, asn, gln, asp, or glu) can be generated. General methods for engineering such N-end rule residues into ubiqtitin-reporter chimera expression vectors are well known in the art (e.g. the “fusion PCR” method; see Karremran, BioTechniques 24: 736-42, 1988).

6. Libraries and Screening Methods

In certain applications of the intrapolypeptide split-ubiquitin conformational assays of the invention, polypeptide and/or small molecule libraries may be utilized. For example, intrapolypeptide split-ubiquitin assays for therapeutic compounds which stabilize or destabilize the conformation of a particular target protein, such as a beta conformation of the beta-amyloid protein, can be performed using small molecule libraries, peptide libraries or nucleic acid expression libraries. Similarly, specific binding polypeptides for a predetermined ligand can be designed by expression of appropriate libraries of variegated split-ubiquitin/polypeptide sequences. Interacting polypeptides can be selected by monitoring split-ubiquitin reporter output from individual clones in the presence and absence of the ligand.

Library Construction

At least two important aspects of library construction need to be considered. One is the source of DNA, the other is the choice of vector suitable for the library.

Many different types of source DNA can be used for library construction. One of the most commonly used source is complementary DNA (cDNA), which is normally obtained by reverse transcription of mRNA isolated from cell lines or tissues, followed by second strand synthesis to complete the synthesis of double-stranded cDNA. The synthesis of cDNA is common knowledge and there are numerous commercially available kits and laboratory manuals covering this subject, and therefore it will not be discussed further.

Genomic DNA (gDNA) is another major source of DNA, although it is less common for construction of an expression library, largely due to the presence of introns and other non-coding regions. The isolation of genomic DNA and size fractionation into suitable pieces for library construction is also well-known in the art.

Other DNA sources can also be used. For example, random or semi-random polynucleotide sequences can be used as source DNA for library construction. This is a particularly powerful method when small stretches of these random fragments are incorporated into a known coding sequence to screen for optimal sequences for certain activity; i.e. binding between two proteins or enzymatic activity.

Many vectors are suitable for library construction. Generally, the chosen vector shall have at least one cloning site for insertion of source DNA. The most commonly used cloning sites are restriction enzyme sites, preferably those restriction enzymes that rarely cut inside coding sequences, such as NotI, SalI. However, other sites can also be used. For example, loxP sites can be used instead of or in addition to restriction enzyme sites. Such sites flanking the cloned source DNA can be recognized by Cre recombinase and readily excised in a controlled manner since Cre recombinase can be conditionally provided by induced expression. Many other similar recombination-based systems are also commercially available, such as the Gateway system (Life Technology, Inc.) that is described in U.S. Pat. No. 5,888,732, the content of which is incorporated by reference herein.

The vector shall also be suitable for expression of the cloned source DNA, either in vitro or in vivo. At the minimum, it shall have a promoter for transcription of the DNA in its intended host. The host can be a mammalian cell, an insect cell, or a plant cell, or any other cell as specified in other sections of this specification. The vector shall also have the ability to maintain itself in the host cell, at least during the pendency of the experiment. That can be achieved by self replication or integration into the host genome. Some vector may also contain selectable markers to facilitate easy identification of cells that have accepted/maintained the vector, and thus the source DNA.

Numerous vectors fit into the definition as outlined above. For example, but without limitation, U.S. Pat. Nos. 5,521,093, 5,538,863, 5,637,504, 5,866,404, and 6,221,588 provide ample examples of yeast vectors suitable for expression of heterologous genes, the contents of which are all incorporated herein in their entirety.

Furthermore, a large number of vectors developed for expression in mammalian cells fulfill the requirements as outlined above. U.S. Pat. No. 6,255,071 has detailed description of a variety of viral vectors suitable for mammalian expression screen, which is incorporated herein by reference in its entirety. Specifically, U.S. Pat. No. 6,255,071 relates to methods and compositions for improved mammalian complementation screening, functional inactivation of specific essential or non-essential mammalian genes, and identification of mammalian genes which are modulated in response to specific stimuli. In particlar, it discloses replication-deficient retroviral vectors, libraries comprising such vectors, retroviral particles produced by such vectors in conjunction with retroviral packaging cell lines, integrated provirus sequences derived from the retroviral particles and circularized provirus sequences which have been excised from the integrated provirus sequences. It further discloses novel retroviral packaging cell lines for use for those viral vectors. Exemplary vectors disclosed by the patent are:

1) A retroviral vector containing a polycistronic message cassette, a proviral excision element for excising retroviral provirus from the genome of a recipient cell and a proviral recovery element for recovering excised provirus from a complex mixture of nucleic acid, a 5′ retroviral long terminal repeat (5′ LTR), a 3′ retroviral long terminal repeat (3′ LTR), a packaging signal, a bacterial origin of replication, and a selectable marker. The retroviral vector may also contain a polycistronic message cassette which makes possible a selection scheme that directly links expression of a selectable marker to transcription of a cDNA or genomic DNA (gDNA) sequence. Such a polycistronic message cassette can comprise, in one embodiment, from 5′ to 3′, the following elements: a nucleotide polylinker, an internal ribosome entry site and a mammalian selectable marker. The polycistronic cassette is situated within the retroviral vector between the 5′ LTR and the 3′ LTR at a position such that transcription from the 5′ LTR promoter transcribes the polycistronic message cassette. The transcription of the polycistronic message cassette may also be driven by an internal cytomegalovirus (CMV) promoter or an inducible promoter, which may be preferable depending on the screenings. The polycistronic message cassette can further comprise a cDNA or genomic DNA (gDNA) sequence operatively associated within the polylinker.

Internal ribosome entry site sequences are well known to those of skill in the art and can comprise, for example, internal ribosome entry sites derived from foot and mouth disease virus (FDV), encephalomyocarditis virus, poliovilus and RDV (Scheper, 1994, Biochemic 76: 801-809; Meyer, 1995, J. Virol. 69: 2819-2824; Jang, 1988, J. Virol. 62: 2636-2643; Haller, 1992, J. Virol. 66: 5075-5086).

Any mammalian selectable marker can be utilized as the polycistronic message cassette mammalian selectable marker. Such mammalian selectable markers are well known to those of skill in the art and can include, but are not limited to, kanamycin/G418, hygromycinB or mycophenolic acid resistance markers. Other examples are provided elsewhere herein.

The retroviral vectors' proviral excision element allows for excision of retroviral provirus (see below) from the genome of a recipient cell. The element comprises a nucleotide sequence which is specifically recognized by a recombinase enzyme. The recombinase enzyme cleaves nucleic acid at its site of recognition in such a manner that excision via recombinase action leads to circularization of the excised nucleic acid molecules.

In a preferred embodiment, the recombinase recognition site is located within the 3′ LTR at a position which is duplicated upon integration of the provirus. This results in a provirus that is flanked by recombinase sites.

In another preferred embodiment, the proviral excision element comprises a loxP recombination site, which is cleavable by a Cre recombinase enzyme. Contacting Cre recombinase to an integrated provirus derived from the retroviral vector results in excision of the provirus nucleic acid. In the alternative, a mutant lox P recombination site may be used (e.g., lox P511 (Hoess et al., 1986, Nucleic Acids Research 14:2287-2300)) that can only recombine with an identical mutant site.

In yet another preferred embodiment, an FRT recombination site, which is cleavable by a FLP recombinase enzyme, is utilized in conjunction with FLP recombinase enzyme, as described above for the loxP/Cre embodiment. In yet an alternative embodiment, a rare-cutting restriction enzyme (e.g., Not I) may be used in place of the recombinase site. The recovered DNA would be digested with Not I and then recircularized with ligase. In this embodiment, the Not I site is included in the vector next to loxP. In still another embodiment, an r recombinase site and r recombinase from Zygosaccharomyces rouxii can be utilized, as described above, for the loxP/Cre embodiment.

In the complementation screening system of the invention, described below, such excision systems can also serve to discriminate revertants from virus-dependent rescue events.

The retroviral vectors' proviral recovery element allows for recovery of excised provirus from a complex mixture of nucleic acid, thus allowing for the selective recovery and excision of provirus from a recipient cell genome. The proviral recovery element comprises a nucleic acid sequence which corresponds to the nucleic acid portion of a high affinity binding nucleic acid/protein pair.

The nucleic acid can include, but is not limited to, a nucleic acid which binds with high affinity to a lac repressor, tet repressor or lambda repressor protein. For example, in one embodiment, the proviral recovery element comprises a lac operator nucleic acid sequence, which binds to a lac repressor peptide sequence. Such a proviral recovery element can be affinity-purified using lac repressor bound to a matrix (e.g., magnetic beads or sepharose). An excised provirus derived from the retroviral vectors of the invention also contains the retroviral recovery element and can be affinity purified.

The 5′ LTR comprises a promoter, including but not limited to an LTR promoter, an R region, a U5 region and a primer binding site, in that order. Nucleotide sequences of these LTR elements are well known to those of skill in the art.

The 3′ LTR comprises a U3 region which comprises the proviral excision element, a promoter, an R region and a polyadenylation signal. Nucleotide sequences of such elements are well known to those of skill in the art.

The bacterial origin of replication (Ori) utilized is preferably one which does not adversely affect viral production or gene expression in infected cells. As such, it is preferable that the bacterial Ori is a non-pUC bacterial Ori relative (e.g., pUC, colEI, pSC101, p15A and the like). Further, it is preferable that the bacterial Ori exhibit less than 90% overall nucleotide similarity to the pUC bacterial Ori. In a preferred embodiment, the bacterial origin of replication is a RK2 OriV or f1 phage Ori.

Any bacterial selectable marker can be utilized. Bacterial selectable markers are well known to those of skill in the art and can include, but are not limited to, kanamycin/G418, zeocin, actinomycin, ampicilln, gentamycin, tetracycline, chloramphenicol or penicillin resistance markers.

The retroviral vectors can further comprise a lethal stuffer fragment which can be utilized to select for vectors containing cDNA or gDNA inserts during, for example, construction of libraries comprising the retroviral vectors of the invention. Lethal stuffer fragments are well known to those of skill in the art (see, e.g., Bernord et al., 1994, Gene 148:71-74, which is incorporated herein by reference in its entirety). A lethal stuffer fragment contains a gene sequence whose expression conditionally inhibits cellular growth.

In one embodiment, the stuffer fragment is present in the retroviral vectors of the invention within the polycistronic message cassette polylinker such that insertion of a cDNA or gDNA sequence into the polylinker replaces the stuffer fragment. Alternatively, the polycistronic message cassette polylinker is located within the lethal stuffer fragment coding sequence such that, upon insertion of a cDNA or gDNA sequence into the polylinker, the lethal stuffer fragment coding region is disrupted. Each of these embodiments can be utilized to counter select retroviral vectors not containing polylinker insertions.

The retroviral vectors can further comprise a single-stranded replication origin, preferably an f1 single-stranded replication origin. The single-stranded replication origin allows for the production of normalized single-stranded retroviral libraries derived from the retroviral vectors of the invention. A normalized library is one constructed in a manner that increases the relative frequency of occurrence of rare clones while decreasing simultaneously the relative frequency of the occurrence of abundant clones. For teaching regarding the production of normalized libraries, see, e.g., Soares et al. (Soares, M. B. et al., 1994, Proc. Natl. Acad. Sci. USA 91:9228-9232, which is incorporated herein by reference in its entirety). Alternative normalization procedures based upon biotinylated nucleotides may also be utilized.

2) A mammalian episomal vector, termed pEHRE vector, which makes possible, stable, efficient, high-level episomal expression within a wide spectrum of mammalian cells. Such vectors can also, for example, be utilized as part of the complementation screening methods of the invention.

Such pEHRE expression vectors comprise a replication cassette, an expression cassette and minimal cis-acting elements necessary for replication and stable episomal maintenance.

The pEHRE vectors can further contain at least one bacterial origin of replication and/or recombination sites. The recombination sites preferably flank the replication cassette, and can include, but are not limited to, any of the recombination sites described above.

Any bacterial origin of replication (Ori) which does not adversely affect the expression of pEHRE sequences can be utilized. For example, the bacterial Ori can be a pUC bacterial Ori relative (e.g., pUC, colEI, pSC101, p15A and the like). The bacterial origin of replication can also, for example, be a RK2 OriV or f1 phage Ori. The pEHRE vectors can further comprise a single stranded replication origin, preferably an f1 single-stranded replication origin. The single-stranded replication origin allows for the production of normalized single-stranded libraries derived from the pEHRE vectors of the invention.

In instances wherein an f1 origin of replication is utilized, the pEHRE vectors can additionally comprise a nucleic acid sequence which corresponds to the nucleic acid portion of a high affinity binding nucleic acid/protein pair. Such nucleic acid/protein pairs can be those as described above, the nucleic acid portion of which can include, but is not limited to, a lacO site. The nucleic acid can include, but is not limited to, a nucleic acid which binds with high affinity to a lac repressor, tet repressor or lambda repressor protein. For example, in one embodiment, the proviral recovery element comprises a lac operator nucleic acid sequence, which binds to a lac repressor peptide sequence. Such a proviral recovery element can be affinity-purified using lac repressor bound to a matrix (e.g., magnetic beads or sepharose). An excised provirus derived from the retroviral vectors of the invention also contains the retroviral recovery element and can be affinity purified.

A pEHRE vector replication cassette comprises nucleic acid sequences which encode papillomaviruses (PV) E1 and E2 proteins, wherein such nucleic acid sequences are operatively attached to and transcribed by, a constitutive transcriptional regulatory sequence. Representative E1 and E2 amino acid sequences are well known to those of skill in the art. See, e.g., sequences publicly available in databases such as Genbank. The E1 and E2 coding sequences can, first, include any nucleotide sequences which encode endogenous PV, including but not limited to bovine papillomavirus (BPV), such as BPV-1 E1 or E2 gene products.

As used herein, the term “E1” also refers to any protein which is capable of functioning in PV in the same manner as the endogenous E1 protein. i.e., is capable of complementing an E1 mutation. Taking BPV as an example, an E1 protein, as described herein, is one capable of complementing a BPV E1 mutation. Likewise, the term “E2”, as used herein, refers to any protein which is capable of functioning in PV in the same manner as the endogenous E2 protein, i.e., is capable of complementing a E2 mutation. Taking BPV as an example, an E2 protein, as described herein, is one capable of complementing a BPV E2 mutation.

The replication cassette constitutive transcriptional regulatory sequence can include, but is not limited to, any polII promoter, such as an SV40, CMV or PGK promoter, nucleotide sequences of which are well known to those of skill in the art.

E1 and E2 coding sequences can be operatively attached to, and transcribed by, separate transcriptional regulatory sequences. In one embodiment, at least one of the E1 or E2 coding sequences can be transcribed along with a selectable marker as a polycistronic message. Such a polycistronic message construction makes possible a selection scheme which directly links expression of a selectable marker, preferably a mammalian selectable marker, to transcription of a sequence necessary for episomal maintenance and replication. For example, the portion of a replication cassette encoding such a polycistronic message could comprise, from 5′ to 3′: a constitutive transcriptional regulatory sequence, an E2 (or E1) coding sequence, an internal ribosome entry site (IRES), and a selectable marker.

In another embodiment, both E1 and E2 coding sequences can be transcribed as a polycistronic message. That is, both E1 and E2 coding sequences, separated by an internal ribosome entry site, can be transcribed by a single transcriptional regulatory sequence.

In yet another embodiment, E1, E2 and selectable marker sequences can be transcribed as a polycistronic message. For example, the replication cassette could comprise, from 5′ to 3′: a constitutive transcriptional regulatory sequence, an E2 (or E1) coding sequence, an IRES, an E1 (or E2) coding sequence, an IRES and a selectable marker.

In instances wherein the E1 and E2 coding sequences are transcribed as part of a polycistronic message, it is preferred that the order, from 5′ to 3′, be E2 then E1. This is to ensure against possible rare, undesirable RNA splicing events.

The pEHRE vector expression cassette is designed to yield high level expression of a cDNA or genomic DNA (gDNA) sequence. Such a pEHRE vector expression cassette comprises, from 5′ to 3′, a transcriptional regulatory sequence, a nucleotide polylinker, an internal ribosome entry site, a mammalian selectable marker and, preferably, either a poly-A site or a transcriptional termination sequence, depending upon the transcriptional regulatory sequence utilized (see below). A cDNA or gDNA sequence can be expressed via operative association within the polylinker. A pEHRE expression vector can contain a single or multiple expression cassettes, such that greater than one cDNA or gDNA sequence can be expressed from the same pEHRE expression vector.

The pEHRE vector expression cassette transcriptional regulatory sequence can be either constitutive or inducible, and can be derived from cellular or viral sources. For example, such transcriptional regulatory sequences can include, but are not limited to, a retroviral long terminal repeat (LTR), cytomegalovirus (CMV), Va-1 RNA or U6 snRNA promoter sequence, nucleotide sequences of which are well known to those of skill in the art. Depending upon the transcriptional regulatory sequence chosen, the expression cassette can contain either a poly A site (pA) or a transcriptional termination sequence. One of skill in the art will readily be able to choose, without undue experimentation, the appropriate sequence to be used with any given transcriptional regulatory sequence. In general, for example, polII-type transcriptional regulatory sequences can be coupled with pA sites, and polII-type transcriptional regulatory sequences can be coupled with transcriptional termination sequences.

Expression from the transcriptional regulatory sequence yields a polycistronic message comprising the cDNA or gDNA sequence of interest, IRES and mammalian selectable marker. Such a polycistronic message approach allows a selection scheme which ensure that the cDNA or gDNA of interest has been expressed.

The pEHRE vectors further comprise cis-acting elements which function in replication and stable episomal maintenance. Such sequences include: a PV minimal origin of replication (MO) and a PV minichromosomal maintenance element (MME). Representative MO and MME sequences are well known to those of skill in the art. See, e.g., Piirson, M. et al., 1996, EMBO J. 15:1-11, which is incorporated herein by reference in its entirety.

As used herein, the term “MO” refers to any nucleotide sequence capable of functioning in PV in the same manner as endogenous MO, i.e., is capable of complementing an MO mutation. Taking BPV as an example, an MO sequence, as described herein, would be one capable of complementing or replacing a BPV MO mutation. Likewise, the term “MME”, as used herein, refers to any nucleotide sequence capable of functioning in PV in the same manner as endogenous MME, i.e., is capable of complementing a MME mutation. For example, a MME sequence can be one containing multiple E2 binding sites. Talking BPV as an example, a MME sequence, as described herein, would be one capable of complementing or replacing a BPV MME mutation.

The pEHRE IRES and mammalian and bacterial selectable markers can be, for example, as those described above.

The pEHRE expression vectors of the invention can be utilized for the production, including large scale production, of recombinant proteins. The vectors' desirable features, in fact, make them especially amenable to large scale production. Specifically, current methods of producing recombinant proteins in mammalian cells involve transfection of cells (e.g., CHO, NS/0 cells) and subsequent amplification of the transfected sequence using drugs (e.g., methotrexate or inhibitors of glutamine synthetase). Such approaches suffer for a variety of reasons, including the fact that amplicons are subject to statistical variation depending on their genomic integration loci, and from the fact that the amplicons are unstable in the absence of continued selection (which is impractical at production scale). The pEHRE vectors, it should be pointed out, achieve such levels equal or higher than these naturally, that is, in the absence of outside selection.

The pEHRE vectors give consistently high episomal expression, making them genomic integration-independent. Further, the episomal pEHRE vectors are retained as stable nuclear plasmids even in the absence of selective pressure.

Further, pEHRE vectors can be utilized which employ an additional level of such internal, or self, selection (that is, selection which does not depend on the addition of outside selective pressures such as, e.g., drugs). For example, pEHRE vectors can be utilized which complement a defect the specific producer cell line being utilized for expression. By way of example, and not by way of limitation, such pEHRE selection elements can complement an auxotrophic mutation or can bypass a growth factor requirement (e.g., proline or insulin, respectively) from the cell media. Preferably, the coding sequence of the marker is transcribed as part of a polycistronic message along with the coding sequence of the proteins being recombinantly expressed. For example, such an expression/selection cassette can comprise, from 5′ to 3′: a transcriptional regulatory sequence, recombinant protein coding sequence, IRES, selection marker, poly-A site.

The episomal pEHRE vectors can further be utilized, for example, in the delivery of large nucleic acid segments, e.g., chromosomal segments. In one such embodiment, pEHRE vectors can be utilized in connection with bacterial artificial chromosome (BAC) or yeast artificial chromosome (YAC) sequences to allow delivery of large genomic segments (e.g., segments ranging from tens of kilobases to megabases in length). For clarity, the discussion that follows describes vectors that utilize BAC sequences, but it is to be understood that vectors of the sort described here can, alternatively, utilize YAC sequences.

In one embodiment, pEHRE vectors can be combined with existing BAC clones to generate pEHRE/BAC hybrid constructs, comprising BACs into which pEHRE vector sequences have been inserted. Such pEHRE/BAC hybrids represent BACs that can replicate in a wide variety of mammalian, including human cells.

In general, pEHRE vectors which can be utilized to donate elements to BACs comprise a pEHRE replication cassette, MO and MME sequences, and a bacterial selectable marker, all flanked by BAC recombination sequences. The remainder of the vector can further comprise at least one bacterial origin of replication and a second bacterial selectable marker.

BAC recombination sequences caN include any nucleotide sequence which can be cleaved and then used to recombine with BAC elements so as to incorporate the necessary pEHRE sequences described above. Any recombination site for which a compatible recombination site exists, or is engineered to exist, in the recipient BAC can be used. For example, such BAC recombination elements can include, but are not limited to, loxP, mutant loxP or frt sites as described above.

Alternatively, CosN sites, whose nucleotide sequences are well known to those of skill in the art, can be utilized. Rather than a recombinase enzyme, such CosN sites are cleaved by lambda terminase enzyme. (For general BAC teaching, including CosN teaching, see, e.g., Shizuya, H. et al., 1992, Proc. Natl. Acad. Sci. USA 89:8794-8797; and Kim, U.-J. et al., 1996, Genomics 34:213-218, which are incorporated herein by reference in their entirety.)

In order to recombine pEHRE and BAC sequences, pEHRE vectors and BAC (containing a recombination site compatible with the chosen pEHRE vector) are treated together with the appropriate recombinase or terminase enzyme. When the CosN/terminase system is used, a subsequent ligation step is included.

The treatment will result in a low level of concatamerization. Concatamers representing the desired pEHRE/BAC hybrids can be selected for based upon their resistance to both the BAC selectable marker (usually chloramphenicol) and the pEHRE vector selectable marker within the pEHRE region meant to be donated. It is, therefore, desirable that the BAC and pEHRE selectable markers be different. In a preferred embodiment, the resulting constructs are further tested to ensure that the second pEHRE bacterial selectable marker is no longer present. Plasmids which have recombined the desired BAC and pEHRE elements, will be able to replicate in E. coli, as well as a wide range of mammalian cells, including human cells.

The vector termed a pBPV-BacDonor vector, represents one embodiment of a pEHRE vector designed to donate essential pEHRE sequences to recipient BAC clones. The vector's recombination elements are depicted as containing loxP and/or CosN sites. The bacterial marker to be incorporated into the pEHRE/BAC hybrid is depicted as tetracycline or kanamycin. Finally, the vector contains a pUC bacterial origin (Ori) of replication, an f1 Ori and a second bacterial selectable marker, ampicillin.

In an alternative embodiment, pEHRE/BAC cloning vectors can be produced and utilized. Such vectors contain the pEHRE replication cassette, MO and MME sequences as described above, the nucleotide sequences necessary for BAC maintenance in E. coli (such sequences are well known to those of skill in the art; see, e.g., Shizuya and Kim, above), and a polylinker site.

The vector termed pBPV-BlueBAC, represents one embodiment of such a pEHRE/BAC cloning vector. In this vector, the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived (in the figure, both of these sequences are illustrated as “BPV origin”). The cloning site comprises a polylinker embedded within the alpha complementation fragment of lacZ, which allows blue/white selection of recombinants. T7 and SP6 promoters flank the lacZ sequence, and the vector additionally contains cosN and loxP sites for linearization. The remainder of the elements depicted are present for BAC maintenance in E. coli.

3) A genetic suppressor element (GSE)-producing replication-deficient retroviral vectors. Such vectors are designed to facilitate the expression of antisense GSE single-stranded nucleic acid sequences in mammalian cells, and can, for example, be utilized in conjunction with the antisense-based functional gene inactivation methods of the invention.

The GSE-producing retroviral vectors can comprise a replication-deficient retroviral genome containing a proviral excision element, a proviral recovery element and a genetic suppressor element (GSE) cassette.

The GSE-producing retroviral vectors can further comprise, (a) a 5′ LTR; (b) a 3′ LTR; (c) a bacterial Ori; (d) a mammalian selectable marker; (e) a bacterial selectable marker; and (f) a packaging signal.

The proviral recovery element, GSE cassette, bacterial Ori, mammalian selectable marker and bacterial selectable marker are located between the 5′ LTR and the 3′ LTR. The proviral excision element is located within the 3′ LTR. The proviral excision element can also flank the functional cassette without being present in the 3′ LTR.

The 5′ LTR, 3′ LTR, proviral excision element, bacterial selectable marker, mammalian selectable marker and proviral recovery element are as described above.

Each of the GSE cassette embodiments described below can further comprise a sense or antisense cDNA or gDNA fragment or full length sequence operatively associated within the polylinker.

The GSE cassette can, for example, comprise, from 5′ to 3′: (a) a transcriptional regulatory sequence; (b) a polylinker; and (c) polyadenylation signal. In one embodiment, the GSE cassette polyadenylation signal is located within the 3′ retroviral long terminal repeat.

Alternatively, the GSE cassette can comprise, from 5′ to 3′: (a) a transcriptional regulatory sequence; (b) a polylinker; (c) a cis-acting ribozyme sequence; (d) an internal ribosome entry site; (e) the mammalian selectable marker; and (f) a polyadenylation signal.

In a further alternative, a sense GSE can be constructed, in which case the GSE cassette can further comprise a polylinker containing a Kozak consensus methionine in front of the sense-orientation fragments to create a “domain library” for domain and fragment expression.

In such an embodiment, transcription from the transcriptional regulatory sequence produces a bifunctional transcript. The first half (i.e., the portion upstream of the ribozyme sequence) is likely to remain nuclear and represents the GSE. The portion downstream of the ribozyme sequence (i.e., the portion containing the selectable marker) is transported to the cytoplasm and translated. Such a bicistronic configuration, therefore, directly links selection for the selectable marker to expression of the GSE.

In another alternative, the GSE cassette can comprise, from 5′ to 3′: (a) an RNA polymerase III transcriptional regulatory sequence; (b) a polylinker; (c) a transcriptional termination sequence. In a particular embodiment, the transcriptional regulatory sequence and transcriptional termination sequence are adenovirus Ad2 VA RNAI transcriptional regulatory and termination sequences.

(4) A genetic suppressor element (GSE)-producing pEHRE vectors. Such vectors are designed to facilitate the expression of antisense GSE single-stranded nucleic acid sequences in mammalian cells, and can, for example, be utilized in conjunction with the antisense-based functional gene inactivation methods of the invention.

The GSE-producing pEHRE vectors of the invention can comprise a replication cassette, a genetic suppressor element (GSE) cassette and minimal cis-acting elements necessary for replication and stable episomal maintenance.

The GSE-producing pEHRE vectors can further comprise at least one bacterial origin of replication and at least one bacterial selectable marker.

The replication cassette, minimal cis-acting elements, bacterial origin of replication and bacterial selectable marker are as described above.

Each of the GSE cassette embodiments described below can further comprise a sense or antisense cDNA or gDNA fragment or full length sequence operatively associated within the polylinker.

The GSE cassette can, for example, comprise, from 5′ to 3′: (a) a transcriptional regulatory sequence; (b) a polylinker; and (c) polyadenylation signal. The GSE transcriptional regulatory sequence can be a constitutive or inducible one, and can represent, for example, retroviral long terminal repeat (LTR), cytomegalovirus (CMV), Va-1 RNA or U6 snRNA promoter sequence, nucleotide sequences of which are well known to those of skill in the art.

A pEHRE GSE vector could, for example be constructed in such a way that the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived. The vector's GSE cassette comprises a CMV promoter operatively associated with a sequence to be expressed as a GSE, which, in turn, is operatively attached to a bgH poly-A site. Finally, the vector contains a pUC bacterial origin (Ori) of replication, an f1 Ori and an ampicillin bacterial selectable marker.

Alternatively, the GSE cassette can comprise, from 5′ to 3′: (a) a transcriptional regulatory sequence; (b) a polylinker; (c) a cis-acting ribozyme sequence; (d) an internal ribosome entry site; (e) the mammalian selectable marker; and (f) a polyadenylation signal.

In another alternative, a sense GSE can be constructed, in which case the GSE cassette can further comprise a polylinker containing a Kozak consensus methionine in front of the sense-orientation fragments to create a “domain library” for domain and fragment expression.

In such an embodiment, transcription from the transcriptional regulatory sequence produces a bifunctional transcript. The first half (i.e., the portion upstream of the ribozyme sequence) is likely to remain nuclear and represents the GSE. The portion downstream of the ribozyme sequence (i.e., the portion containing the selectable marker) is transported to the cytoplasm and translated. Such a bicistronic configuration, therefore, directly links selection for the selectable marker to expression of the GSE.

In another alternative, the GSE cassette can comprise, from 5′ to 3′: (a) an RNA polymerase III transcriptional regulatory sequence; (b) a polylinker; (c) a transcriptional termination sequence.

In a particular embodiment the transcriptional regulatory sequence and transcriptional termination sequence are adenovirus Ad2 VA RNA transcriptional regulatory and termination sequences.

(5) A vector useful for the display of constrained and unconstrained random peptide sequences. Such vectors are designed to facilitate the selection and identification of random peptide sequences that bind to a protein of interest.

The retroviral and pEHRE vectors displaying random peptide sequences of the present invention can comprise, (a) a splice donor site or a LoxP site (e.g., LoxP511 site); (b) a bacterial promoter (e.g., pTac) and a shine-delgarno sequence; (c) a pel B secretion signal for targeting fusion peptides to the periplasm; (d) a splice-acceptor site or another LoxP511 site (Lox P511 sites will recombine with each other, but not with the LoxP site in the 3′ LTR); (e) a peptide display cassette or vehicle; (f) an amber stop codon; (g) the M13 bacteriophage gene 111 protein C-terminus (amino acids 198-406); and optionally the vector may also comprise a flexible polyglycine linker.

A peptide display cassette or vehicle consists of a vector protein, either natural or synthetic into which a polylinker has been inserted into one flexible loop of the natural or synthetic protein. A library of random oligonucleotides encoding random peptides may be inserted into the polylinker, so that the peptides are expressed on the cell surface.

The display vehicle of the vector may be, but is not limited to, thioredoxin for intracellular peptide display in mammalian cells (Colas et al., 1996, Nature 380:548-550) or may be a minibody (Tramonteno, 1994, J. Mol. Recognit. 7:9-24) for the display of peptides on the mammalian cell surface. Each of these would contain a polylinlxer for the insertion of a library of random oligonucleotides encoding random peptides at the positions specified above. In an alternative embodiment, the display vehicle may be extracellular, in this case the minibody could be preceded by a secretion signal and followed by a membrane anchor, such as the one encoded by the last 37 amino acids of DAF-1 (Rice et al.; 1992, Proc. Natl. Acad. Sci. 89:5467-5471). This could be flanked by recombinase sites (e.g., FRT sites) to allow the production of secreted proteins following passage of the library through a recombinase expressing host.

In one embodiment of the present invention, these cassettes would reside at the position normally occupied by the cDNA in the sense-expression vectors described above. In an amber suppressor strain of bacteria and in the presence of helper phage, these vectors would produce a relatively conventional phage display library which could be used exactly as has been previously described for conventional phage display vectors. Recovered phage that display affinity for the selected target would be used to infect bacterial hosts of the appropriate genotype (i.e., expressing the desired recombinases depending upon the cassettes that must be removed for a particular application). For example for an intracellular peptide display, any bacterial host would be appropriate (provided that splice sites are used to remove pelB in the mammalian host). For a secreted display, the minibody vector would be passed through bacterial cells that catalyze the removal of the DAF anchor sequence. Plasmids prepared from these bacterial hosts are used to produce virus for assay of specific phenotypes in mammalian cells.

The advantage of these vectors over conventional approaches is their flexibility. The ability to functionally test the peptide sequence in mammalian cells without additional cloning or sequencing steps males possible the use of much cruder binding targets (e.g., whole fixed cells) for phage display. This is made possible by the ability to do a rapid functional selection on the enriched pool of bound phages by conversion to retroviruses that can infect mammalian cells. (6) A replication-deficient retroviral gene trapping vector. Such gene trapping vectors contain reporter sequences which, when integrated into an expressed gene, “tag” the expressed gene, allowing for the monitoring of the gene's expression, for example, in response to a stimulus of interest. The gene trapping vectors of the invention can be used, for example, in conjunction with the gene trapping-based methods of the invention for the identification of mammalian genes which are modulated in response to specific stimuli.

The replication-deficient retroviral gene trapping vectors of the invention can comprise: (a) a 5′ LTR; (b) a promoterless 3′ LTR (a SINS LTR); (c) a bacterial Ori; (d) a bacterial selectable marker; (e) a selective nucleic acid recovery element for recovering nucleic acid containing a nucleic acid sequence from a complex mixture of nucleic acid; (f) a polylinker; (g) a mammalian selectable marker; and (h) a gene trapping cassette. In addition, those elements necessary to produce a high titer virus are required. Such elements are well known to those of skill in the art and contain, for example, a packaging signal.

The bacterial Ori, bacterial selectable marker, selective nucleic acid recovery element, polylinker, and mammalian selectable marker are located between the 5′ LTR and the 3′ LTR. The bacterial selectable marker and the bacterial Ori are located in close operative association in order to facilitate nucleic acid recovery, as described below. The gene trapping cassette element is located within the 3′ LTR.

The 5′ LTR, bacterial selectable marker and mammalian selectable marker are as described above. The selective nucleic acid recovery element is as the proviral recovery element described above.

The 3′ LTR contains the gene trapping cassette and lacks a functional LTR transcriptional promoter.

The gene trapping cassette can comprise from 5′ to 3′: (a) a nucleic acid sequence encoding at least one stop codon in each reading frame; (b) an internal ribosome entry site; and (c) a reporter sequence. The gene trapping cassette can further comprise, upstream of the stop codon sequences, a transcriptional splice acceptor nucleic acid sequence.

The inclusion of the IRES sequence in the gene trapping vectors of the present invention offers a key improvement over conventional gene trapping vectors. The IRES sequence allows the vector to land anywhere in the mature message to create a bicistronic transcript, this effectively increases the number of integration sites that will report promoters by a factor of at least 10. Although some of the vectors disclosed by U.S. Pat. No. 6,255,071 are intended for use in mammalian cells, with minor modification, most can be adepted for use in other cell types. Especially when specific packaging cells are used to generate viruses with a wide spectrum of infection.

Since these libraries are to be used for expression of Nux fusion proteins, a Nux coding sequence shall be present in the vector. Depending on specific configurations of the fusion protein, the Nux coding sequence could be either at the 5′- or 3′-end of the cloning site(s) for source DNA.

A normalized library is one constructed in a manner that increases the relative frequency of occurrence of rare clones while decreasing simultaneously the relative frequency of the occurrence of abundant clones. For teaching regarding the production of normalized libraries, see, e.g., Soares et al. (Soares, M. B. et al., 1994, Proc. Natl. Acad. Sci. USA 91:9228-9232, which is incorporated herein by reference in its entirety). Alternative normalization procedures based upon biotinylated nucleotides may also be utilized.

Those of ordinary skill in the art will recognize that methods for vector construction and protein expression described above and/or provided in the examples are examplary. It should be understood that there are other techniques, vectors, and cell lines that could be implemented for constructing and expressing proteins or fragments thereof in either procaryotic or eukaryotic systems. The preferred embodiment disclosed herein does not limit the scope of the invention. There are a variety of alternative techniques and procedures available to those with ordinary skill in the art that would permit one to perform modifications on the present invention. It is also well-known in the art that commercially available kits allow the modification and incorporation of the present invention. It is further recognized that those with ordinary skill in the art could employ any of a number of known techniques to modify the nucleic acid molecules of the present invention, in vitro or in vivo, and develop them further by established protocols for gene transfer and expression.

Screen Methods

Although examplary mammalian cell complementation screening methods are described herein, it should be understood that many aspects of the described methods can be easily adapted for use in other cell types, which will be apparent to the person of ordinary skill in the art. Complementation screens in certain other cell types, especially in yeast, are well-known in the art. A classic example is genetic analysis of the cell cycle in the budding yeast S. cerevisiae (see review by Hartwell, L. H., Twenty-five years of cell cycle genetics, in Genetics 129: 975-980, 1991). Associated technologies such as yeast tranformation and overexpression of heterologous genes in yeast are well-known in the art and will not be addressed furher. Furthermore, knowledge based on yeast complementation screens has been adapted for use in cross-species complementation screens, for example, in yeast for plant (Arabidopsis) genes (Gietz. D. et al., Nucl. Acids Res. 20: 1425, 1992; Schiestl, R. H. and Gietz, R. D., Curr. Genet. 16: 339-334, 1989), the details of which will not be discussed further.

Nevertheless, complementation screens in mammalian cells constitute one of the most important aspects of the invention. Such complementation screen-methods can include, for example, a method for identification of a nucleic acid sequence whose expression complements a cellular phenotype, comprising: (a) infecting a mammalian cell exhibiting the cellular phenotype with a, for example, retrovirus particle derived from a cDNA or gDNA-containing retroviral vector of the invention, or, alternatively, transfecting such a cell with a pEHRE vector of the invention wherein, depending on the vector, upon infection an integrated retroviral provirus is produced or upon transfection an episomal sequence is established, and the cDNA or gDNA sequence is expressed; and (b) analyzing the cell for the phenotype, so that suppression of the phenotype identifies a nucleic acid sequence which complements the cellular phenotype. Specifically, when a Nux-fusion protein is expressed at the presence of P-Cub-n-RM, interaction between P and the polypeptide encoded as a Nux-fusion will result in the generation of n-RM, which can then be detected depending on the specific nature of the reportermoiety and the nature of the amino acid n. Phenotypic differences between an uncleaved and cleaved n-RM shall allow selection of cells comprising cleaved n-RM.

Isolation and Characterization of Positive Clones

The vectors used mast also facilitate the cloning and further characterization of the encoded polypeptide in the selected cell(s). Such methods utilize the proviral excision and the proviral recovery elements described above.

In one embodiment of such a method, the proviral excision element comprises a loxP recombination site present in two copies within the integrated provirus, and the proviral recovery element comprises a lacO site, present in the provirus between the two loxP sites. In this embodiment, the loxP sites are cleaved by a Cre recombinase enzyme, yielding an excised provirus which, upon excision, becomes circularized. The excised, circular provirus, which contains the lacO site is recovered from the complex mixture of recipient cell genomic nucleic acid by lac repressor affinity purification. Such an affinity purification is made possible by the fact that the lacO nucleic acid specifically binds to the lac repressor protein.

In an alternative embodiment, the excised provirus is amplified in order to increase its rescue efficiency. For example, the excised provirus can further comprise an SV40 origin of replication such that in vivo amplification of the excised provirus can be accomplished via delivery of large T antigen. The delivery can be made at the time of recombinase administration, for example.

In another alternative embodiment, the excised provirus may be recovered by use of a Cre recombinase. For example, the isolated DNA is fragmented to a controlled size. The provirus containing fragments are isolated via LacO/LacI. Following IPTG elution, circularization of the provirus can be accomplished by treatment with purified recombinase. The person skilled in the art will be able to anticipate other methods to isolate and characterize nucleic acids from selected cells.

Variegated Peptide Display

The variegated peptide libraries of the subject method can be generated by any of a number of methods, and, though not limited by, preferably exploit recent trends in the preparation of cheniical libraries. The library can be prepared, for example, by either synthetic or biosynthetic approaches, and screened for activity against the D-enantiomer target in a variety of assay formats. As used herein, “variegated” refers to the fact that a population of peptides is characterized by having a peptide sequence which differ from one member of the library to the next. For example, in a given peptide library of n amino acids in length, the total number of different peptide sequences in the library is given by the product of where each nn represents the number different amino acid residues occurring at position n of the peptide. In a preferred embodiment of the present invention, the peptide display collectively produces a peptide library including at least 96 to 10⁷ different peptides, so that diverse peptides may be simultaneously assayed for the ability to interact with the target protein.

Peptide libraries are systems which simultaneously display, in a form which permits interaction with a target protein, a highly diverse and numerous collection of peptides. These peptides may be presented in solution (Houghten, BioTechniques 13: 412-421, 1992), or on beads (Lam, Nature 354: 82-84, 1991), chips (Fodor, Nature 364: 555-556, 1993), bacteria (Ladner U.S. Pat. No. 5,223,409), spores (Ladner U.S. Pat. No. 5,223,409), plasmids (Cull et al., Proc Natl Acad Sci USA 89: 1865-1869, 1992) or on phage (Scott and Smith, Science 249: 386-390, 1990; Devlin, Science 249: 404-406, 1990; Cwirla et al., Proc. Natl. Acad. Sci. 87: 6378-6382, 1990; Felici, J. Mol. Biol. 222: 301-310, 1991; and Ladner U.S. Pat. No. 5,223,409).

In one embodiment, the peptide library is derived to express a combinatorial library of peptides which are not based on any known sequence, nor derived from cDNA. That is, the sequences of the library are largely random. It will be evident that the peptides of the library may range in size from dipeptides to large proteins.

In another embodiment, the peptide library is derived to express a combinatorial library of peptides which are based at least in part on a known polypeptide sequence or a portion thereof (not a cDNA library). That is, the sequences of the library is semi-random, being derived by combinatorial mutagenesis of a known sequence(s). See, for example, Ladner et al. PCT publication WO 90/02909; Garrard et al., PCT publication WO 92/09690; Marks et al., J. Biol. Chem. 267: 16007-16010, 1992; Griffths et al., EMBO J 12: 725-734, 1993; Clackson et al., Nature 352: 624-628, 1991; and Barbas et al., PNAS 89: 4457-4461, 1992. Accordingly, polypeptide(s) which are known ligands for a target protein can be mutagenized by standard techniques to derive a variegated library of polypeptide sequences which can further be screened for agonists and/or antagonists.

In still another embodiment, the combinatorial polypeptides are produced from a cDNA library.

Depending on size, the combinatorial peptides of the library can be generated as is, or can be incorporated into larger fusion proteins. The fusion protein can provide, for example, stability against degradation or denaturation, as well as a secretion signal if secreted. In an exemplary embodiment, the polypeptide library is provided as part of thioredoxin fusion proteins (see, for example, U.S. Pat. Nos. 5,270,181 and 5,2992,646 and PCT publication WO94/02502). The combinatorial peptide can be attached on the terminus of the thioredoxin protein, or, for short peptide libraries, inserted into the so-called active loop.

In preferred embodiments, the combinatorial polypeptides are in the range of 3-100 amino acids in length, more preferably at least 5-50, and even more preferably at least 10, 13, 15, 20 or 25 amino acid residues in length. Preferably, the polypeptides of the library are of uniform length. It will be understood that the length of the combinatorial peptide does not reflect any extraneous sequences which may be present in order to facilitate expression, e.g., such as signal sequences or invariant portions of a fusion protein.

Biosynthetic Peptide Libraries

The harnessing of biological systems for the generation of peptide diversity is now a well established technique which can be exploited to generate the peptide libraries of the subject method. The source of diversity is the combinatorial chemical synthesis of mixtures of oligonucleotides. Oligonucleotide synthesis is a well-characterized chemistry that allows tight control of the composition of the mixtures created. Degenerate DNA sequences produced are subsequently placed into an appropriate genetic context for expression as peptides.

There are two principal ways in which to prepare the required degenerate mixture. In one method, the DNAs are synthesized a base at a time. When variation is desired at a base position dictated by the genetic code a suitable mixture of nucleotides is reacted with the nascent DNA, rather than the pure nucleotide reagent of conventional polynucleotide synthesis. The second method provides more exact control over the amino acid variation. First, trinucleotide reagents are prepared, each trinucleotide being a codon of one (and only one) of the amino acids to be featured in the peptide library. When a particular variable residue is to be synthesized, a mixture is made of the appropriate trinucleotides and reacted with the nascent DNA. Once the necessary “degenerate” DNA is complete, it must be joined with the DNA sequences necessary to assure the expression of the peptide, as discussed in more detail below, and the complete DNA construct must be introduced into the cell.

Whatever the method may be for generating diversity at the codon level, chemical synthesis of a degenerate gene sequence can be carried out in an automatic DNA synthesizer, and the synthetic genes can then be ligated into an appropriate gene for expression. The purpose of a degenerate set of genes is to provide, in one mixture, all of the sequences encoding the desired set of potential test peptide sequences. The synthesis of degenerate oligonucleotides is well known in the art (see for example, Narang, Tetrahedron 39:3 1983; Itakura et al., Recombinant DNA, Proc 3rd Cleveland Sympos. Macromolecules, ed. A G Walton, Amsterdam: Elsevier pp273-289, 1981; Itakura et al. Annu. Rev. Biochem. 53: 323, 1984; Itakura et al., Science 198: 1056, 1984; Ike et al., Nucleic Acid Res. 11: 477, 1983). Such techniques have been employed in the directed evolution of other proteins (see, for example, Scott et al., Science 249: 386-390, 1990; Roberts et al., PNAS 89: 2429-2433, 1992; Devlin et al., Science 249: 404-406, 1990; Cwirla et al., PNAS 87: 6378-6382, 1990; as well as U.S. Pat. Nos. 5,223,409, 5,198,346, and 5,096,815).

Because the number of different peptides one can create by this combination approach can be huge, and because the expectation is that peptides with the appropriate structural characteristics to serve as ligands for a given target protein will be rare in the total population of the library, the need for methods capable of conveniently screening large numbers of clones is apparent. Several strategies for selecting peptide ligands from the library have been described in the art and are applicable to certain embodiments of the present method.

In one embodiment, a variegated peptide library can be expressed by a population of display packages to form a peptide display library. With respect to the display package on which the variegated peptide library is manifest, it will be appreciated from the discussion provided herein that the display package will often preferably be able to be (i) genetically altered to encode a test peptide, (ii) maintained and amplified in culture, (iii) manipulated to display the peptide in a manner permitting the peptide to interact with a target protein during an affinity separation step, and (iv) affinity separated while retaining the peptide-encoding gene such that the sequence of the peptide can be obtained. In preferred embodiments, the display remains viable after affinity separation.

Ideally, the display package comprises a system that allows the sampling of very large variegated peptide display libraries, rapid sorting after each affinity separation round, and easy isolation of the peptide-encodinig gene from purified display packages. The most attractive candidates for this type of screening are prokaryotic organisms and viruses, as they can be amplified quickly, they are relatively easy to manipulate, and large number of clones can be created. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages.

In addition to commercially available kits for generating phage display libraries (e.g. Pharmacia Recombinant Phage Peptide System, catalog no. 27-9400-01; and the Stratagene SurfZAP™ phage display kit, catalog no. 240612), examples of methods and reagents particularly amenable for use in generating the variegated peptide display library of the present method can be found in, for example, the Ladner et al. U.S. Pat. No. 5,223,409, the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; the Ladner et al. International Publication No. WO 90/02809; Fuchs et al., Bio. Technology 9: 1370-1372, 1991; Hay et al., Hum Antibod Hybridomas 3: 81-85, 1992; Huse et al., Science 246: 1275-1281, 1989; Griffths et al., EMBO J 12: 725-734, 1993; Hawkins et al., J Mol Biol 226: 889-896, 1992; Clackson et al., Nature 352: 624-628, 1991; Gram et al., PNAS 89: 3576-3580, 1992; Garrad et al., Bio/Technology 9: 1373-1377, 1991; Hoogenboom et al., Nuc Acid Res 19: 4133-4137, 1991; and Barbas et al., PNAS 88: 7978-7982, 1991.

As will be apparent to those skilled in the art, in embodiments wherein high affinity peptides are sought, an important criteria for the present selection method can be that it is able to discriminate between peptides of different affinity for a particular target, and preferentially enrich for the peptides of highest affinity. Applying the well known principles of affinity and valence, it is understood that manipulating the display package to be rendered effectively monovalent can allow affinity enrichment to be carried out for generally higher binding affinities (i.e. binding constants in the range of 10⁶ to 10¹⁰ M⁻¹) as compared to the broader range of affinities isolable using a multivalent display package. To generate the monovalent display, the natural (i.e. wild-type) form of the surface or coat protein used to anchor the peptide to the display can be added at a high enough level that it almost entirely eliminates inclusion of the peptide fusion protein in the display package. Thus, a vast majority of the display packages can be generated to include no more than one copy of the peptide fusion protein (see, for example, Garrad et al., Bio/Technology 9: 1373-1377, 1991). In a preferred embodiment of a monovalent display library, the library of display packages will comprise no more than 5 to 10% polyvalent displays, and more preferably no more than 2% of the display will be polyvalent, and most preferably, no more than 1% polyvalent display packages in the population. The source of the wild-type anchor protein can be, for example, provided by a copy of the wild-type gene present on the same construct as the peptide fusion protein, or provided by a separate construct altogether.

Nucleic Acid Libraries

In another embodiment, the library is comprised of a variegated pool of nucleic acids, e.g. single or double-stranded DNA or ARNA. A variety of techniques are known in the art for generating screenable nucleic acid libraries which may be exploited in the present invention. In particular, many of the techniques described above for synthetic peptide libraries can be used to generate nucleic acid libraries of a variety of formats. For example, divide-couple-recombine techniques can be used in conjugation with standard nucleic acid synthesis techniques to generate bead immobilized nucleic acid libraries.

In another embodiment, solution libraries of nucleic acids can be generated which rely on PCR techniques to amplify for sequencing those nucleic acid molecules which selectively bind the screening target. By such techniques, libraries approaching 10¹⁵ different nucleotide sequences have been generated in solution (see, for example, Bartel and Szostak, Science 261: 1411-1418, 1993; Bock et al., Nature 355: 564, 1992; Ellington et al., Nature 355: 850-852, 1992; and Oliphant et al., Mol Cell Biol 9: 2944-2949, 1989).

According to one embodiment of the subject method, the SELEX (systematic evolution of ligands by exponential enrichment) is employed with the enantiomeric screening target. See, for example, Tuerk et al., Science 249: 505-510, 1990, for a review of SELEX. Briefly, in the first step of these experiments on a pool of variant nucleic acid sequences is created, e.g. as a random or semi-random library. In general, an invariant 3′ and (optionally) 5′ primer sequence are provided for use with PCR anchors or for permitting subcloning. The nucleic acid library is applied to screening a target, and nucleic acids which selectively bind (or otherwise act on the target) are isolated from the pool. The isolates are amplified by PCR and subcloned into, for example, phagemids. The phagemids are then transfected into bacterial cells, and individual isolates can be obtained and the sequence of the nucleic acid cloned from the screening pool can be determined.

When RNA is the test ligand, the RNA library can be directly synthesized by standard organic chemistry, or can be provided by in vitro translation as described by Tuerk et al., supra. Likewise, RNA isolated by binding to the screening target can be reverse transcribed and the resulting cDNA subcloned and sequenced as above.

Small Molecule Libraries

Recent trends in the search for novel pharmacological agents have focused on the preparation of chemical libraries. Peptide, nucleic acid, and saccharide libraries are described above. However, the field of combinatorial chemistry has also provided large numbers of non-polymeric, small organic molecule libraries which can be employed in the subject method.

Exemplary combinatorial libraries include benzodiazepines, peptoids, biaryls and hydantoins. In general, the same techniques described above for the various formats of chemically synthesized peptide libraries are also used to generate and (optionally) encode synthetic non-peptide libraries.

Selecting Compounds from the Library

As with the diversity contemplated for the screening target and form in which the compound library is provided, the subject method is envisaged with a variety of detection methods for isolating and identifying compounds which interact with the screening target. In most embodiments, the screening programs which test libraries of compounds will be derived for high throughput analysis in order to maximize the number of compounds surveyed in a given period of time. However, as a general rule, the screening portion of the subject method involves contacting the screening target with the compound library and isolating those compounds from the library which interact with the screening target. Such interaction may be detected, for example, based on directly detecting the binding of the compounds to the screening target, or inferred through the modulation of interactions involving the screening target with other molecules, such as protein-protein or protein-DNA interaction involving the screening target or modulation of an enzymatic/catalytic activity of the screening target. The efficacy of the test compounds can be assessed by generating dose response curves from data obtained using various concentrations of the test compound. Moreover, a control assay can also be performed to provide a baseline for comparison.

Complex formation between a test compounds and a screening target may be directly detected by a variety of techniques. The complexes can be scored for using, for example, detectable labeled compounds or screening targets, such as radiolabeled, fluorescently labeled, or enzymatically labeled polypeptides, by immunoassay, or by chromatographic detection.

In one embodiment, the variegated compound library is subjected to affinity enrichment in order to select for compounds which bind a preselected screening target. The term “affinity separation” or “affinity enrichment” includes, but is not limited to (1) affinity chromatography utilizing immobilizing screening targets, (2) precipitation using screening targets, (3) fluorescence activated cell sorting where the compound library is so amenable, (4) agglutination, and (5) plaque lifts. In each embodiment, the library of compounds are ultimately separated based on the ability of a particular compound to bind a screening target of interest. See, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; and the Ladner et al. International Publication No. WO 90/02809.

With respect to affinity chromatography, it will be generally understood by those skilled in the art that a great number of chromatography techniques can be adapted for use in the present invention ranging from column chromatography to batch elution and including ELISA and reverse biopanning techniques. Typically the screening target is immobilized on an insoluble carrier, such as sepharose or polyacrylamide beads, or, alternatively, the wells of a microtitre plate.

The population of compounds is applied to the affinity matrix under conditions compatible with the binding of compounds in the library to the immobilized screening target. The population is then fractionated by washing with a solute that does not greatly effect specific binding of compounds to the screening target, but which substantially disrupts any non-specific binding of components the library to the screening target or matrix. A certain degree of control can be exerted over the binding characteristics of the compounds recovered from the library by adjusting the conditions of the binding incubation and subsequent washing. The temperature, pH, ionic strength, divalent cation concentration, and the volume and duration of the washing can select for compounds within a particular range of affinity and specificity. Selection based on slow dissociation rate, which is usually predictive of high affinity, is a very practical route. This may be done either by continued incubation in the presence of a saturating amount of free screening target, or by increasing the volume, number, and length of the washes. In each case, the rebinding of dissociated compounds from the applied library is prevented, and with increasing time, compounds of higher and higher affinity are recovered. Moreover, additional modifications of the binding and washing procedures may be applied to find compounds with special characteristics. The affinities of some compounds may be dependent on ionic strength or cation concentration. Specific examples are peptides which depend on Ca⁺⁺ or other ions for binding activity and which release from the screening target in the presence of a chelating agent such as EGTA. (see, Hopp et al., Biotechnology 6: 1204-1210, 1988). Such peptides may be identified in the compound library by a double screening technique isolating first those that bind the screening target in the presence of Ca⁺⁺, and by subsequently identifying those in this group that fail to bind in the presence of EGTA.

After “washing” to remove non-specifically members of the compound library, when desired, specifically compounds can be eluted by either specific desorption (using excess screening target) or non-specific desorption (using pH, polarity reducing agents, or chaotropic agents). In preferred embodiments using biological display packages, the elution protocol does not kill the organism used as the display package such that the enriched population of display packages can be further amplified by reproduction. The list of potential eluants includes salts (such as those in which one of the counter ions is Na⁺, NH₄⁺, Rb⁺, SO₄²⁻, H₂PO₄⁻, citrate, K⁺, Li⁺, Cs⁺, HSO₄⁻, CO₃²⁻, Ca²⁺, Sr²⁺, Cl⁻, PO₄²⁻, HCO₃⁻, Mg²⁺, Ba²⁺, Br⁻, HPO₄²⁻, or acetate), acid, heat, and, when available, soluble forms of the target antigen (or analogs thereof). Because bacteria continue to metabolize during the affinity separation step and are generally more susceptible to damage by harsh conditions, the choice of buffer components (especially eluates) can be more restricted when the display package is a bacteria rather than for phage or spores. Neutral solutes, such as ethanol, acetone, ether, or urea, are examples of other agents useful for eluting the bound display packages.

In preferred embodiments of biological peptide displays or certain nucleic acid libraries, affinity enriched packages or nucleic acids are iteratively amplified and subjected to further rounds of affinity separation until enrichment of the desired binding activity is detected. In certain embodiments, the specifically bound biological display packages, especially bacterial cells, need not be eluted per se, but rather, the matrix bound display packages can be used directly to inoculate a suitable growth media for amplification.

Where the display package is a phage particle, the fusion protein generated with the coat protein can interfere substantially with the subsequent amplification of eluted phage particles, particularly in embodiments wherein the cpIII protein is used as the display anchor. Even though present in only one of the 5-6 tail fibers, some peptide constructs because of their size and/or sequence, may cause severe defects in the infectivity of their carrier phage. This causes a loss of phage from the population during reinfection and amplification following each cycle of panning. In one embodiment, the peptide can be derived on the surface of the display package so as to be susceptible to proteolytic cleavage which severs the covalent linkage of at least the antigen binding sites of the displayed peptide from the remaining package. For instance, where the cpIII coat protein of M13 is employed, such a strategy can be used to obtain infectious phage by treatment with an enzyme which cleaves between the peptide portion and cpIII portion of a tail fiber fusion protein (e.g. such as the use of an enterokinase cleavage recognition sequence).

To further minimize problems associated with defective infectivity, DNA prepared from the eluted phage can be transformed into host cells by electroporation or well known chemical means. The cells are cultivated for a period of time sufficient for marker expression, and selection is applied as typically done for DNA transformation. The colonies are amplified, and phage harvested for a subsequent round(s) of panning.

After isolation of biological display packages which encode peptides having a desired binding specificity for the screening target, the nucleic acid encoding the peptide for each of the purified display packages can be recloned in a suitable eukaryotic or prokaryotic expression vector and transfected into an appropriate host for production of large amounts of protein.

On the other hand, where chemically synthesized libraries are used in the form of display packages, the isolated peptides are identified either directly from the display, e.g., by direct inicrosequencing, or the display packages are appropriately decoded, e.g., by elucidating the identity of an associated tag/index. Deconvolution techniques are also known in the art.

It will be apparent that, in addition to utilizing binding as the separation criteria, compound libraries can be fractionated based on other activities of the target molecule, such as modulation of catalytic activity.

7. Therapeutic Formulations

The intrapolypeptide split-ubiquitin therapeutic formulations used in the method of the invention are most preferably applied in the form of appropriate compositions. As appropriate compositions there may be cited all compositions usually employed for systemically or topically administering drugs. The pharmaceutically acceptable carrier should be substantially inert, so as not to act with the active component. Suitable inert carriers include water, alcohol, polyethylene glycol, mineral oil or petroleum gel, propylene glycol and the like.

8. Transgenic Animals

In certain instances, it may be desirable to engineer stable mammalian cell lines expressing the N_{ub} and C_{ub} chimeric fusion polypeptide in order to facilitate screening applications of the invention. Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a target gene of interest in ES cells, these changes can be introduced into the germlines of animals to generate chimeras. The gene targeting procedure is accomplished by introducing into tissue culture cells a DNA targeting construct that includes a segment homologous to a target gene locus, and which also includes an intended sequence modification to the target genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a target gene function through the use of a targeting transgene construct designed to undergo homologous recombination with one or more target genomic sequences. The targeting construct can be arranged so that, upon recombination with an element of a target gene, a positive selection marker is inserted into (or replaces) coding sequences of the gene. The inserted sequence functionally disrupts the target gene, while providing a positive selection trait. Exemplary target gene targeting constructs are described in more detail below.

Generally, the embryonic stem cells (ES cells) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al., J. Embryol. Exp. MoMFGFhol. 87: 27-45, 1985). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (Ioffe et al., PNAS 92: 7357-7361, 1995). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C., 1987); by Bradley et al., Current Topics in Devel. Biol 20: 357-371, 1986); and by Hogan et al. (Manipulating the Mouse Embryo: A Laboratory Manual, Cold Spring Harbor Laboratory Press, Cold Spring Harbor, N.Y., 1986).

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for target gene or other gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgeric animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., β-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the above-mentioned knock out and knock in constructs so that they are flanked by negative selectable markers at either end (articularly through the use of two allelic variants of the thymidine kinase gene, the polyeptide product of which can be selected against in expressing cell lines in an appropriate tissue culture medium well known in the art—i.e. one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker. Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the lnockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (Sura).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line canies the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the MFGF gene knocked out in various tissues of the offspring by probing the Western blot with an antibody against the particular MFGF protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a target-gene can be controlled by recombinase sequences (described infra).

Animals containing more than one knockout construct and/or more than one transgene expression construct are prepared in any of several ways. The preferred manner of preparation is to generate a series of mamnmals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the lcockout construct(s) and/or transgene(s).

A target transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a target gene protein can be essential for many forms of lineage analysis and can additionally provide a means to assess the effects of, for example, lack of target gene expression which might grossly alter development in small patches of tissue within an otherwise normal embryo. Toward this and, tissue-specific regulatory sequences and conditional regulatoiy sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes can be regulated via site-specific genetic manipulation in vitro, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject target gene proteins. For example, excision of a target sequence which interferes with the expression of a recombinant target gene, such as one which encodes an antagonistic homolog or an antisense transcript, can be designed to activate expression of that gene. This interference with expression of the protein can result from a variety of mechanisms, such as spatial separation of the target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenlic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the crelloxP recombinase system of bacteriophage P1 (Lakso et al., PNAS 89: 6232-6236, 1992; Orban et al., PNAS 89: 6861-6865, 1992) or the FLP recombinase system of Saccharomyces cerevisiae (O'Gorman et al., Science 251: 1351-1355, 1991; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al., J. Biol. Chem. 259: 1509-1514, 1984); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatery control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant target gene protein can be regulated via control of recombinase expression.

Use of the crelloxP recombinase system to regulate expression of a recombinant target gene protein requires the construction of a transgenic animal containing transgenes encoding both the Cre recombinase and the subject protein. Animals containing both the Cre recombinase and a recombinant target gene can be provided through the construction of “double” transgenic animals. A convenient method for providing such animals is to mate two transgenic animals each containing a transgene, e.g., a target gene and recombinase gene.

One advantage derived from initially constructing transgenic animals containing a target transgene in a recombinase-mediated expressible format derives from the likelihood that the subject protein; whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic target transgene is silent will allow the study of progeny from that founder in which disruption of target gene mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the target transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, a target transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for micro-injection. In the mouse, the male pronucleus reaches the size of approximately 20 μm in diameter which allows reproducible injection of 1-2 μL of DNA solution. The use of zygotes as a target for gene transfer has a major advantage in that in most cases the injected DNA will be incorporated into the host gene before the first cleavage (Brinster et al., PNAS 82:4438-4442, 1985). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protaniines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, wlilch is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatile, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic materials preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgeilic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a target protein (either agonistic or antagonistic), and antisense transcript, or a target mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, PNAS 73: 1260-1264, 1976). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al., PNAS 82: 6927-6931, 1985; Van der Putten et al., PNAS 82: 6148-6152, 1985). Transfection is easily and efficiently obtained by culturing the blastomeres on a monolayer of virus-producing cells (Van der Putten, supra; Stewart et al., EMBO J 6: 383-388, 1987). Alternatively, infection can be performed at a later stage. Virus or virus-producing cells can be injected into the blastocoele (Jahner et al., Nature 298: 623-628, 1982). Most of the founders will be mosaic for the transgene since incorporation occurs only in a subset of the cells which formed the transgeric non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al., 1982, supra).

A third type of target cell for transgene introduction is the embryonal stem cell (ES). ES cells are obtained from pre-implantation embryos cultured in vitro and fused with embryos (Evans et al., Nature 292: 154-156, 1981; Bradley et al., Nature 9: 255-258, 1984; Gossler et al., PNAS 83: 9065-9069, 1986; and Robertson et al., Nature 322: 445-448, 1986). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenich, Science 240: 1468-1474, 1988.

9. Types of Conformation Changes

The instant invention provides a method to detect or screen for polypeptide conformation changes in response to a variety of reasons.

The intrapolypeptide split-ubiquitin therapeutic formulations used in the method of the invention are most preferably applied in the form of appropriate compositions. As appropriate compositions there may be cited all compositions usually employed for systemically or topically administering drugs. The pharmaceutically acceptable carrier should be substantially inert, so as not to act with the active component. Suitable inert carriers include water, alcohol, polyethylene glycol, mineral oil or petroleum gel, propylene glycol and the like.

For example, several p53 mutations in the core domain are shown in Example 3 below to cause conformation change in the core domain, which is readily detectable by the split-ubiquitin system of the instant invention.

A second type of conformation change of the polypeptide can be caused by binding of the polypeptide to an agent or compound that is not covalently linked to the polypeptide. The nature of the agent/compound varies. It can be a polypeptide, a hormone, a steroid, an ion, a polynucleotide, a sugar or an oligosaccharide, a lipid, an enzyme substrate, O₂, and a small molecule.

Protein-protein interaction may lead to conformation change of at least one interacting partners. For example, as shown in the example below, yeast Gβ binding to Gγ causes a conformation change in the Gγ protein, which can be readily detected by the method of the instant invention.

Hormone induced conformation change is well known in the art. For example, the nuclear hormone receptor estrogen receptor (ER) contains a ligand-binding domain (LBD) that is completely enveloped by other protein domains. Thus, the process of ligand binding or unbinding must involve a significant conformational change of this domain.

As to conformational change induced by small chemical compounds or drugs, Connor et al. (Cancer Res. 61: 2917-22, 2001) report that an anti-estrogen chemical GW5638 induces a unique structural change in the estrogen receptor (ER). It is known that tamoxifen inhibits estrogen receptor transcriptional activity by competitively inhibiting estradiol binding and inducing conformational changes in the receptor that may prevent interaction with coactivators. In bone, the cardiovascular system, and some breast tumors, however, tamoxifen exhibits agonist activity, suggesting that the tamoxifen-ER complex is not recognized identically in all cells. Using phage display, Connor et al. demonstrate that the anti-estrogen GW5638 induces a unique structural change in the ER. The biological significance of this conformational change was revealed in studies that demonstrated that tamoxifen-resistant breast tumor explants are not cross-resistant to GW5638. Thus, this drug can potentially be used as a therapeutic for tamoxifen-resistant breast cancers.

Steroid-induced changes in receptor protein conformation constitute a logical means of translating the variations in steroid structures into the observed array of whole cell biological activities. One conformational change in the rat glucocorticoid receptor (GR) can be readily discerned by following the ability of trypsin digestion to afford a 16-kDa fragment. This fragment is seen after proteolysis of steroid-free receptors but disappears in digests of either glucocorticoid- or antiglucocorticoid-bound receptors (Xu et al. Mol. Cell. Endocrinology 155: 85-100, 1999).

Ion binding may also change protein conformation. Fur (ferric uptake regulation protein) is a bacterial global regulator that uses iron as a cofactor to bind to specific DNA sequences. It has been suggested that metal binding induces a conformational change in the protein, which is subsequently able to recognize DNA. Gonzalez de Peredo et al. used selective chemical modification and mass spectrometry to investigate this mechanism of conformation change. The reactivity of each lysine residue of the Fur protein was studied, first in the apo form of the protein, then after metal activation and finally after DNA binding. Of particular interest is Lys76, which was shown to be highly protected from modification in the presence of target DNA. Hydrogen-deuterium exchange experiments were performed to map with higher resolution the conformational changes induced by metal binding. On the basis of these results, together with a secondary structure prediction, the presence in Fur of a non-classical helix-turn-helix motif is proposed. Experimental results show that activation upon metal binding induces conformational modification of this specific motif. The recognition helix, interacting directly with the major groove of the DNA, would include the domain [Y55-F61]. This helix would be followed by a small “wing” formed between two beta strands, containing Lys76, which might interact directly with DNA.

DNA-induced conformation change in certain transcription factors is well known in the art. Although bacteriophage 434 repressor binds to its specific DNA sites only as a dimer, formation of the dimers in solution occurs at concentrations three orders of magnitude higher than those needed to bind the 434 operator DNA. Ciubotaru et al. show that both specific and non-specific DNA may induce conformational changes that can lead to formation of repressor dimers (J. Mol. Biol. 294: 859-873, 1999). The repressor conformational changes induced by DNA occur at concentrations much lower than those needed for binding of repressor, suggesting that the alternative conformations of repressor persist even if the protein is not in direct contact with DNA. Hence, DNA acts in a “catalytic” fashion to induce a steady-state amount of an alternative repressor conformation that has an enhanced affinity for its specific binding site. These findings suggest that the repressor conformer induced by non-specific DNA is form of the repressor that is optimized for searching for DNA binding sites along non-specific DNA. Upon finding a binding site, the repressor protein undergoes an additional conformational change that allows it to “lock-on” to its specific site.

Lipid-protein interaction may also induce conformation change. The interaction of apolipoprotein H (Apo H) with lipid membrane has been considered to be a basic mechanism for the biological function of the protein. Previous reports have demonstrated that Apo H can interact only with membranes containing anionic phospholipids. Wang et a. study the membrane-induced conformational change of Apo H by CD spectroscopy with two different model systems: anionic-phospholipid-containing liposomes [such as 1, 2-dimyristoyl-sn-glycero-3-phosphoglycerol (DMPG) and cardiolipin], and the water/methanol mixtures at moderately low pH, which mimic the micro-physicochemical environment near the membrane surface. It is found that Apo H undergoes a remarkable conformational change on interaction with liposomes containing anionic phospholipid. To interact with liposomes containing DMPG, there is a 6.8% increase in alpha-helix in the secondary structures; in liposomes containing cardiolipin, however, there is a 12.6% increase in alpha-helix and a 9% decrease in beta-sheet. The similar conformation change in Apo H can be induced by treatment with an appropriate mixture of water/methanol. The results indicate that the association of Apo H with membrane is correlated with a certain conformational change in the secondary structure of the protein.

A key element in the ability of lac repressor protein to control transcription reversibly is the capacity to assume different conformations in response to ligand (lactose and other structural homologs of the sugar, such as IPTG) binding. To investigate regions of the protein involved in these conformational changes, Barry and Matthews investigated mutant repressor proteins containing single tryptophans created by mutating each of the two native tryptophan residues to tyrosine and changing the residue of interest to tryptophan (Biochemistry 36: 15632-42, 1997). This study suggests that, in the areas of the lac repressor probed by those substitutions, the inducer-bound form differs from the conformation of the unliganded form.

Gas can also change protein conformation. One well documented examples is conformation change in hemoglobin (Hb) after binding to oxygen or CO. Quaternary structure of Hb is a tetramer with 2 alpha and 2 beta subunits and they are labelled alpha-1, alpha-2, beta-1 and beta-2. They join together in pairs of alpha-1+beta-1 and alpha-2+beta-2 because the interactions between these pairings are much stronger than e.g. between alpha-1 and alpha-2. The interactions are salt bridges or electrostatic interactions, and all 4 are connected to each other by these electrostatic contacts. Examination of the structure showed that deoxy Hb has 8 more contacts (salt bridges) between the subunits than has oxy Hb. So deoxy Hb is a tighter, more rigid molecule.

When Hb binds oxygen it undergoes a change in conformation, which disrupt the salt bridges. When all 4 oxygen molecules have bound, all 8 salt bridges are disrupted; so oxy Hb is more relaxed, held together loosely. A change in conformation arises when oxygen binds to Fe and pulls the Fe into the plane of the haem. This movement pulls on the His F8 residue (the eight residue in helix F. The Fe atom is located very slightly above the haem plane, and attached to the His F8 side), and flattens the haem plane, and these movements cause a series of small changes in orientation of the amino acid involved in salt bridges between subunits so that the salt bridges break. So by small alterations in the molecular shape or conformation of the Hb this facilitates rapid additional binding of oxygen.

Enzyme-substrate interaction frequently, if not always, induces conformation change of the enzyme (and/or substrate). In glycolysis, binding of glucose to hexokinase induces a large conformational change of the enzyme (a phenomenon also referred to as “induced fit”). The change in conformation brings the C6 hydroxyl of glucose close to the terminal phosphate of ATP, and excludes water from the active site. This prevents the enzyme from catalyzing ATP hydrolysis, rather than transfer of phosphate to glucose.

A third type of protein conformation change can be induced by environmental alterations, such as temperature, pressure, pH, redox state, etc.

Temperature change can induce conformation change in proteins. Numerous temperature sensitive (including cold sensitive) mutant proteins have been described. These mutants typically adopt a defective conformation at higher temperature (or lower temperature in the cold sensitive case) and return to a relatively normal conformation at a lower temperature.

Pressure has also been documented to induce conformation change and protein denaturation. Weingand-Ziade et al. studied the effect of pressure and temperature on the inactivation process of native (N) human butyrylcholinesterase (BuChE) and found that it to be a multi-step process. It led to irreversible formation of an active intermediate (I) state and a denatured state. This series-inactivation process was described by expanding the Lumry-Eyring [Lumry, R. and Eyring, H. (1954) J. Phys. Chem. 58, 110-120] model. The intermediate state (I) was found to have a Kₘ identical with that of the native state and a turnover rate k_{cat} twofold higher than that of the native state with butyrylthiocholine as the substrate. The increased catalytic efficiency (k_{cat}/Kₘ) of (I) can be explained by a conformational change in the active-site gorge and/or restructuring of the water-molecule network in the active-site pocket, making the catalytic steps faster.

Changes in pH can also induce protein conformation alteration. Ceramide can induce apoptosis through a caspase independent pathway. Bax has been described as able to kill cells in the absence of caspase activity, therefore Belaud-Rotureau et al. measured Bax in situ during ceramide-induced apoptosis using anti-Bax antibodies and flow cytometry analysis (Apoptosis 5: 551-560, 2000). An early (<30 min) increase in Bax labeling was observed after the addition of several ceramide species to several hemopoietic-related cell types. On U937, this increase was not due to antigens synthesis or processing, but rather an increased accessibility or reactivity of Bax antigens for antibodies. This increased immuno-reactivity of Bax was not inhibited by Z-VAD-fmk nor leupeptin, and preceded nuclear fragmentation by several hours. Such an increase in immuno-reactivity was also observed after Fas ligation, but it occurred later (>2 h) accompanying nuclear apoptosis, and was inhibited by Z-VAD-fmk. Bax immuno-reactivity was found to be related to intracellular pH (pHi), and C2-Ceramide (C2-Cer) induced a very early (<10 min) transitory increase in pHi. Both Bax immunoreactivity and pHi increases were dependent on the mitochondrial permeability transition pore (PTP) status. It was concluded from these results that C2-Cer induced a transitory increase in pHi in relation to the PTP. This rise in pHi led to conformational changes in Bax which could be responsible for further apoptosis in the C2-Cer pathway while it was a consequence of caspase activation in the Fas pathway.

Protein conformation may also change in response to redox changes. The Escherichia coli OxyR transcription factor senses H₂O₂ and is activated through the formation of an intramolecular disulfide bond. Choi et al. recently reported the crystal structures of the regulatory domain of OxyR in its reduced and oxidized forms, determined at 2.7 Å and 2.3 Å resolutions, respectively (Cell 105: 103-113, 2001). In the reduced form, the two redox-active cysteines are separated by approximately 17 Å. Disulfide bond formation in the oxidized form results in a significant structural change in the regulatory domain. The structural remodeling, which leads to different oligomeric associations, accounts for the redox-dependent switch in OxyR an provides a novel example of protein regulation “fold editing” through a reversible disulfide bond formation within a folded domain.

A fourth type of protein conformation change can be induced by post-translational modification, such as phosphorylation, acetylation, methylation, glycosylation, proteolytic cleavage, sulfation, hydroxylation, carboxylation and prenylation, etc.

Phosphorylation/dephosphorylation are one of the most important mechanisms in signal transduction. There are numerous examples of phosphorylation induced protein conformation changes. For example, Lee et al. studied phosphorylation induced conformation change in NADPH oxidase (Biochimie 82: 727-32, 2000). The leukocyte NADPH oxidase of neutrophils is a membrane-bound enzyme that catalyzes the production of O²⁻ from oxygen using NADPH as the electron donor. During activation, the cytosolic oxidase components p47(phox) and p67(phox), each containing two Src homology 3 (SH3) domains, migrate to the plasma membrane, where they associate with cytochrome b(558), a membrane-integrated flavohemoprotein, to assemble the active oxidase. Oxidase activation can be mimicked in a cell-free system using an anionic amphiphile, such as SDS or arachidonic acid and the phosphorylation of p47(phox )with protein kinase C, activators of the oxidase in vitro cause exposure of p47(phox)-SH3, which has probably been masked by the C-terminal region of this protein in a resting state. Lee et al. show that the total protein steady-state intrinsic fluorescence exhibited by the tryptophan residues of p47(phox) substantially decreased when N-terminal truncated p47(phox)-SH3-C was treated with anionic amphiphiles or phosphorylated with protein kinase C. This finding was similar to the results obtained with full-length p47(phox). However, the fluorescence of C-terminal truncated p47(phox)-N-SH3 and both C-terminal and N-terminal truncated p47(phox)-SH3 were not altered by the activators. These results indicate that the C-terminal region of p47(phox) is a primary target of the conformational change during the activation of NADPH oxidase.

Acetylation is an important mechanism in regulating conformation change of proteins. For example, modification of histones, DNA-binding proteins found in chromatin, by addition of acetyl groups occurs to a greater degree when the histones are associated with transcriptionally active DNA. A breakthrough in understanding how this acetylation is mediated was the discovery that various transcriptional co-activator proteins have intrinsic histone acetyltransferase activity (for example, Gen5p, PCAF, TAF(II)250 and p300/CBP. These acetyl transferases also modify certain transcription factors (TFIIEbeta, TFIIF, EKLF and p53). GATA-1 is an important transcription factor in the haematopoietic lineage and is essential for terminal differentiation of erythrocytes and megakaryocytes. It is associated in vivo with the acetyltransferase p300/CBP. Boyes et al. report that GATA-1 is acetylated in vitro by p300 (Nature 396: 594-8, 1998). This significantly increases the amount of GATA-1 bound to DNA and alters the mobility of GATA-1-DNA complexes, suggestive of a conformational change in GATA-1. GATA-1 is also acetylated in vivo and acetylation directly stimulates GATA-1-dependent transcription. Mutagenesis of important acetylated residues shows that there is a relationship between the acetylation and in vivo function of GATA-1. Thus, acetylation induced conformation change in transcription factors can alter interactions between these factors and DNA and among different transcription factors, and is an integral part of transcription and differentiation processes.

Methylation may also change protein conformation. The Escherichia coli protein Ada specifically repairs the S(p) diastereomer of DNA methyl phosphotriesters in DNA by direct and irreversible transfer of the methyl group to its own Cys69 which is part of a zinc-thiolate center. The methyl transfer converts Ada into a transcriptional activator that binds sequence-specifically to promoter regions of its own gene and other methylation resistance genes. Ada thus acts as a chemosensor to activate repair mechanisms in situations of methylation damage. Lin et al. report a highly refined solution structure of the 10 kDa N-terminal domain, N-Ada10, which reveals structural details of the nonspecific DNA interaction of N-Ada10 during the repair process and provides a basis for understanding the mechanism of the conformational switch triggered by methyl transfer (Biochemistry 40: 4261-71, 2001). Results from that study show that methylation of N-Ada induces a structural change, which enhances the promoter affinity of a remodeled surface region that does not include the transferred methyl group.

Glycosylation induced conformation change is also reported. Tagashira et al. synthesized seven O-glycosylated calcitonin derivatives, each with a single GalNAc residue attached to either Ser or Thr, and studied their three-dimensional structure and biological activity to examine site-dependent effects of O-glycosylation (Biochemistry 40: 11090-5, 2001). The CD spectra in an aqueous trifluoroethanol solution showed that the GalNAc attachment at Thr6 or Thr21 reduced the helical content of calcitonin, indicating that the O-glycosylated residue functions as a stronger helix breaker than the original amino acid residue. Only the GalNAc attachment at Ser2 or Thr21 retained the hypocalcemic activity of calcitonin. This result corresponded well to that of the calcitonin-receptor binding assay. The GalNAc attachment other than Ser2 or Thr21 perturbed the interaction with the receptor, resulting in the loss of the hypocalcemic activity. The biodistribution did not change much among the seven derivatives, but some site dependency could also be observed. Thus, they conclude that the O-glycosylation affects both the conformation and biological activity in a site-dependent manner.

It is apparent from the above description that protein conformation changes can be induced by a variety of stimuli, including mutational alteration, binding by a compound in trans, post-translational modification, and environmental alterations. It shall be understood that these examples listed above are for exemplification of various possible conformation changes that can be detected according to the instant invention. However, it shall not be construed to be limiting in any sense.

### EXAMPLES

### Example 1

Introduction

The maturation, conformational stability, and the rate of in vivo degradation are specific for each protein and depend on both the intrinsic features of the protein and those of the surrounding cellular environment. While synthesis and degradation can be measured in living cells, stability and maturation of proteins are more difficult to quantify. We developed the split-ubiquitin method into a tool for detecting and analyzing changes in protein conformation. The biophysical parameter that forms the basis of these measurements is the time-averaged distance between the N-terminus and C-terminus of a protein. Starting from three proteins of known structure, we demonstrate in the following examples the feasibility of this approach, and employ it to elucidate the effect of a previously described mutation in the protein Sec62p on its conformation in living cells. Furthermore, we demonstrate use of fusion proteins of the invention to investigate the effect of an alteration in the environmental factor temperature, on protein confirmation of a Sec62p mutation.

### Example 1.1

Split-Ubiquitin Intrapolypeptide Assay for N- to C-Terminus Distance

In order to determine whether the split-ubiquitin assay could be used to measure the distance between the amino-terminus and the carboxy-terminus of a fully-folded polypeptide in its mature conformation, we created a unique N_{ub}-polypeptide-C_{ub}-n-Reporter construct, wherein n was an amino-acid residue capable of destabilizing the reporter when the reporter is released as an n-RM following cleavage of the N_{ub}-polypeptide-C_{ub}-n-RM by a ubiquitin-specific protease (UBP). For example, if the protein normally folds so as to bring amino- and carboxy-termini into close proximity, then the N_{ub} and C_{ub} halves will frequently associate causing UBPs to cleave and release the destabilized reporter (n-reporter) thereby resulting in relatively low levels of reporter moiety and/or activity. In contrast, if the two ends of the polypeptide normally fold so as to result in a great distance between the amino- and carboxy-termini of the protein, then the two ubiquitin halves will remain unassociated, the reporter will not be released by UBPs and relatively high levels of reporter moiety and/or activity will result. Furthermore, protein conformation of the polypeptides can be further measured with this intrapolypeptide split-ubiquitin assay in either of these two situations. In the first scenario, conformational alterations in the intervening polypeptide will inevitably decrease the association of N_{ub} and C_{ub} ends, thereby decreasing the rate of n-Reporter release by UBPs and increasing the level of reporter activity. In the second scenario, conformational alterations result in a potential increase in the rate of N_{ub}/C_{ub} association, thereby increasing the rate of UBP-mediated release of n-reporter and so decreasing the level of the measurable reporter moiety and/or activity level. Therefore this intrapolypeptide split-ubiquitin assay can be used to study the conformation of virtually any protein of interest regardless of whether the mature folded form of the particular protein results in close proximity of amino- and carboxy-termini.

Structural analysis revealed for most proteins a fixed distance between the N-terminus and the C-terminus. Significant changes in the conformation of a protein can be expected to alter this distance. By attaching N_{ub} to the N-terminus and C_{ub} to the C-terminus of a single polypeptide, we tried to exploit the efficiency of the N_{ub}—C_{ub} reassociation to measure changes in the time-averaged distance between the N- and the C-terminus. To test this concept, we first analyzed two yeast proteins with known structures. The structure of Guk1p, the guanylate kinase of the yeast S. cerevisiae, brings the N- and C-terminus in close proximity (Stehle et al., J. Mol. Biol. 224: 1127-41, 1992). Guk1p in a N_{ub}-Guk1-C_{ub} fusion protein should therefore facilitate the reassociation of the Ub-peptides. The structure of Fpr1p, the yeast FKBP12 homologue, spatially separates the N- from the C-terminus (Rotonda et al., J. Biol. Chem. 268: 7607-09, 1993). Fpr1p in a N_{ub}-Fpr1-C_{ub} fusion protein should therefore inhibit the Ub-reassembly (FIGS. 1a, b). These predictions should only apply to the fully folded state of the two proteins. Compared to the corresponding wild type protein, mutations or conditions that destabilize the structure of Guk1p should reduce the N_{ub}—C_{ub} reassociation of a N_{ub}-Guk1-C_{ub} fusion, whereas mutations or conditions that destabilize the structure of Fpr1p should enhance the N_{ub}—C_{ub} reassociation of a N_{ub}-Fpr1-C_{ub} fusion protein (FIG. 1c).

### Example 1.2

Spatial Arrangement of the N- and C-Termini Influences Reassembly of Coupled Ub-peptides

In applying the above-described fusion proteins as proof of principle of the intrapolypeptide split-ubiquitin assay, we first determined the optimal N_{ub} and C_{ub} sequences to use in the reassociation study. In particular, completely wild-type sequences, when fused to a polypeptide that brings amino- and carboxy-termini into close proximity, might associate so strongly that subsequent decreases with conformational unfolding would be difficult to detect.

We first constructed a set of N_{ub}-mutants that each displayed a different affinity for C_{ub}. The isoleucine residues at position three and thirteen of N_{ub} (Nᵢᵢ) were replaced by valine (Nₓᵥ; Nᵥₓ) alanine (Nₓₐ; Nₐₓ) and glycine (N_{xg}; N_{gx}) (Johnsson et al., Proc. Natl. Acad. Sci. USA 91: 10340-44, 1994; Kellis et al., Biochemistry 28: 4914-22, 1989). The residues at position 3 and 13 are indicated by the suffix in N_{ub} with the corresponding amino acids in the single letter code. The sixteen different combinations were first tested in the otherwise native Ub which was attached in front of the dihydrofolate reductase carrying the HA epitope at its C-terminus (Ub-Dha). The analysis of the cleaved and uncleaved protein was performed by immunodetection of the proteins with the anti-HA antibody after cell extraction and SDS-PAGE separation. Complete cleavage of the Ub-Dha was observed for all mutants except for those carrying the glycine at position 3 and an alanine or a glycine at position 13 and those that carry an alanine at position 3 and a glycine at position 13 (FIG. 2a, only N_{gi}, N_{ga} and N_{gg} are shown).

A subset of 12 of these new N_{ub} was fused in front of Guk1-F—C_{ub}-Dha, where F encodes 16 additional residues containing the FLAG epitope (F). The cleavage pattern of the 12 different N_{ub}-Guk1-F—C_{ub}-Dha fusion proteins is shown in FIG. 2b. The fusion proteins were aligned according to the decreasing affinity of the different N_{ub} for the C_{ub}. Compared to the cleavage of the Ub-Dha carrying no insert between N_{ub} and C_{ub}, the reassembly of the corresponding Guk1p fusion proteins is clearly impaired. Starting from 100% cleaved Nᵢᵢ-Guk1-F—C_{ub}-Dha, the uncleaved fusion protein accumulates according to the expected decrease in the affinity between N_{ub} and C_{ub}. Approximately 90% of uncleaved protein is obtained for the N_{gg}-construct. The first detectable uncleaved fusion protein carries the N_{ig}- at its N-terminus.

Unlike Guk1p, the structure of Fpr1p separates N- and C-terminus onto opposite faces of thee molecule (Rotonda et al., J. Biol. Chem. 268: 7607-09, 1993). FPR1 was cloned between eight different N_{ub} and C_{ub}-Dha. The length of the linker that connects the Ub-moieties to the inserted protein slightly influences the reassembly reaction (compare FIGS. 2b and 3a, and our unpublished observation). The FLAG-epitope was therefore omitted in the constructs of this and all the following experiments. The cleavage spectrum was again examined after cell extraction and immunoblot analysis (FIG. 3). Compared to the spectrum of N_{ub}-Guk1-C_{ub}-Dha, the structure of Fpr1p shifts the appearance of the first uncleaved fusion protein to the N_{ub} with an higher affinity for C_{ub} (FIGS. 3a and 3b). Tie Nᵢₐ-construct already reveals a small fraction of uncleaved fusion protein that reaches a surplus with the N_{vg}-construct. No uncleaved product is detected for the corresponding N_{vg}-Guk1-C_{ub}Dha (FIG. 3a). Since Fpr1p is 73 residues smaller than Guk1p, size can not account for the difference in the reassociation rates. Using the appropriate yeast strains, it was also shown that N_{ub}-Fpr1-C_{ub}-Dha as well as the corresponding Guk1p fusion protein code for at least partially functional molecules (see Material and Methods and our unpublished observation). We therefore conclude that the different arrangements of the N- and C-termini in the structures of the two proteins cause the differences in the extent of cleavage of their corresponding N_{ub}—C_{ub} fusion proteins.

### Example 1.3

Controlling for Inter-Molecular Interactions

A significant extent of the observed cleavage could arise from the intermolecular reassociation of two Ub-halves that are linked to two different polypeptides. To estimate the contribution of the intermolecular Ub-reassociation, Guk1-C_{ub}-Dha was coexpressed with several versions of N_{ub}-Guk1-ha as was Fpr1-C_{ub}-Dha with several versions of N_{ub}-Fpr1-ha (FIG. 4a). Good intermolecular N_{ub}—C_{ub} reassociation is only seen in cells expressing the Nᵢᵢ-fusions, whereas none or only background cleavage is seen in those cells co-expressing the corresponding N_{ig}-fusions. N_{ub}-mutants with a lower affinity for C_{ub} than N_{ig} were therefore not tested. It thus can be concluded that the cleaved Dha observed for N_{ig}—C_{ub} fusion proteins and for those fusion proteins containing a N_{ub} with a lower affinity for C_{ub} than N_{ig} must result exclusively from the intramolecular reassociation of the coupled Ub-peptides. The structure of Guk1p offers a reasonable explanation for this observation, that can not apply for the Fpr1p fusion proteins. Here the reassociation must either occur before Fpr1p is folded or after the unfolding of the already matured protein. To approach this issue, cells that expressed N_{vg}-Fpr1-C_{ub}-Dha were pulsed with ³⁵S-methionine for five minutes and chased with cold methionine for 30 and 60 minutes (FIG. 4b). A significant fraction of the uncleaved fusion protein is processed to the cleaved Dha during the chase. We conclude that the fraction of cleaved Dha which is detected in the immunoblot analysis of cells expressing N_{vg}-Fpr1-C_{ub}-Dha is largely derived from fusion proteins which stayed in the cytosol long enough to give the Fpr1-moiety sufficient time to fold.

### Example 1.4

Monitoring Polypeptide Unfolding In Vivo

In many proteins the transition from the folded to the unfolded state should be accompanied by a significant change in the mean distance between the N-terminus and the C-terminus. Since the proportion of unfolded molecules can not be artificially increased by denaturing chemicals or heat in live cells, we attempted to simulate the unfolded state of Fpr1p by exchanging a small, but structurally important part of the protein. The C-terminal sequence of Fpr1p was altered from V₁₀₇ELLKVN₁₁₃(SEQ ID No. 1) to R₁₀₇RIVEGQ₁₁₃(SEQ ID No. 2) to create Fpr1MC. The affected residues constitute the central strand of the antiparallel beta-sheet and the introduced changes should either prevent Fpr1p from folding or reduce the stability of its structure (Rotonda et al., J. Biol. Chem. 268: 7607-09, 1993). N_{ub}-Fpr1MC—C_{ub}-Dha displays a cleavage spectrum that lies between those of N_{ub}-Fpr1-C_{ub}-Dha and N_{ub}-Guk1-C_{ub}-Dha, respectively (FIG. 3c). Uncleaved N_{ub}-Fpr1MC—C_{ub}-Dha is first detected for the N_{ig}-construct. N_{ig} has a weaker affinity for C_{ub} than Nᵢₐ, the first N_{ub} that allows the accumulation of the uncleaved Fpr1p fusion protein, but has a stronger affinity for Nₐᵢ, the first N_{ub} that yields uncleaved Guk1p fusion protein (FIG. 3d).

### Example 1.5

Characterizing Destabilizing Mutations In Vivo

The difference between the 20% of cleavage of the wild type N_{vg}-Fpr1p-C_{ub}-Dha and the 60% of cleavage of N_{vg}-Fpr1MC—C_{ub}-Dha indicated N_{vg} as an appropriate N_{ub}-mutant to sense more subtle changes in the structure of Fpr1p (FIG. 3b). The side chain of valine 107 protrudes into the hydrophobic core of the molecule. Exchanging this valine against the charged arginine (Fpr1V107R) should decrease the structural stability of Fpr1p. The effect of this exchange is reflected in our assay by the increase in the fraction of cleaved N_{vg}-Fpr1V107R—C_{ub}-Dha to 61% (FIG. 5a). To measure a destabilizing, but less destructive mutation, the side chain of valine 107 was reduced by two and three methyl groups in Fpr1V107A and Fpr1V107G, respectively. Both mutations increase the cleavage of the corresponding N_{vg}—C_{ub}-constructs. The 55% of cleavage of the alanine mutant indicate a more severe effect on the stability of Fpr1p than the measured 40% of the glycine mutant (FIG. 5a).

Destabilizing mutations in Guk1p must decrease rather than increase the efficiency of cleavage of the corresponding N_{ub}—C_{ub} fusion proteins. This prediction was confirmed. The first nine N-terminal residues of Guk1p include its N-terminal beta-strand (Stehle et al., J. Mol. Biol. 224: 1127-41, 1992). A deletion of these nine residue increases the fraction of uncleaved fusion protein in the corresponding N_{vg}-Guk1ΔN—C_{ub}-Dha (FIG. 5b).

Replacing valine 5 against an arginine to create Guk1V5R is equivalent to the mutation Fpr1V107R. The decrease in the amount cleaved Guk1V5R fusion construct from 100% to 61% documents a higher proportion of unfolded molecules due to this mutation (FIG. 5b). Exchanges which do not alter the structure or its stability should be silent in this assay. We replaced glycine 8 by an arginine to create Guk1G8R. The corresponding N_{vg}-Guk1G8R—C_{ub}-Dha construct is cleaved as completely as the corresponding wild type construct (FIG. 5b). In accordance with the structure of Guk1p, we assume that the side chain of this arginine does not point into the hydrophobic core, but protrudes into the cleft that is part of the substrate binding pocket (Stehle et al., J. Mol. Biol. 224: 1127-41, 1992). However, since we did not follow the cleavage of this mutant in other N_{ub}—C_{ub}-Dha constructs, the detection of a slight destabilization of the structure might have been missed.

Deleting the helix that constitutes a loop on the surface of Guk1p (residues 58-66; Guk1ΔH) shows no significant alteration in the cleavage of the N_{vg}—C_{ub} construct. N_{vg}-Guk1ΔH—C_{ub}-Dha is still cleaved to more than 95% (FIG. 5b). Here we increased the sensitivity of the assay by switching from N_{vg} to Nₐₐ in the fusion construction. The amount of cleaved Dha is 55% for Nₐₐ-Guk1-C_{ub}-Dha, but only 37% for Nₐₐ-Guk1ΔH—C_{ub}-Dha (data not shown).

### Example 1.6

Defining the Borders of Protein Domains

The classical approach to define autonomous domains in proteins of unknown structure is limited proteolysis. To test whether split-Ub can complement existing methods, we extended our analysis to the human Ub-like protein Sumo1p, the structure of which was solved recently (Bayer et al., J. Mol. Biol. 280: 275-86, 1998). Like their Ub-counterparts, Sumo1-fusion proteins are cleaved at their C-terminal glycine by Sumo1p specific proteases (Johnson et al., Embo. J. 16: 5509-19, 1997). To facilitate our analysis, we repressed this cleavage by deleting the C-terminal extension and the critical glycine at position 97 (residues 97-101; referred to as Sumo1p in this article). This deletion should not affect the central domain of the protein, which separates the N- and C-termini on opposite faces of the structure. Unlike Fpr1p, the N-terminus of Sumo1p is connected to the central domain by a highly flexible 18 residue extension (Bayer et al., J. Mol. Biol. 280: 275-86, 1998). As a consequence, the Fpr1p-like arrangement of the N- and the C-terminus in Sumo1p is not confirmed by our analysis. The 50% cleavage of the N_{vg}-Sumo1-C_{ub}-Dha is closer to the cleavage of N_{vg}-Fpr1MC—C_{ub}-Dha than to the 22% of cleavage of the native Fpr1 fusion protein (FIG. 5c). Deleting this flexible linker in N_{vg}-Sumo 1ΔN—C_{ub}-Dha reduces the cleavage to 15%. This value is close to the value of the Fpr1p fusion and reflects the arrangement of the N-terminus and C-terminus in the central domain of Sumo1p. Deletion of the C-terminal strand (residues 85-95) in Sumo1p (Sumo1ΔC) or Sumo1ΔN (Sumo1ΔNΔC) should destroy the structure of this central domain. As a consequence the extent of cleavage of N_{vg}-Sumo1ΔNΔC—C_{ub}-Dha is approximately as high as the cleavage of N_{vg}-Sumo1-C_{ub}-Dha, N_{vg}-Sumo1ΔC—C_{ub}-Dha, or N_{vg}-Fpr1MC—C_{ub}-Dha (FIG. 5c). In summary, the cleavage pattern of the three different Sumo1 fusion proteins clearly follows the domain structure of the protein (FIG. 5c). The decrease in cleavage after deleting the N-terminal peptide (Sumo1ΔN) marks the beginning of the compact domain. The sharp increase in cleavage caused by the simultaneous deletion of the C-terminal strand (Sumo1ΔNΔC) marks the C-terminal border.

### Example 1.7

Linking Protein Stability to a Selectable Phenotype

Using RUra3p instead of Dha as reporter moiety allowed us to monitor the efficiency of the intramolecular N_{ub—C}_{ub} reassociation by testing the growth of the construct-transformed cells on plates lacking uracil. Upon cleavage, RUra3p is rapidly degraded by the N-end rule pathway. Provided that the reassociation is very efficient, the cells should become uracil auxotroph and 5-FOA resistant (FIG. 6a) (Wittke et al., Mol. Biol. Cell 10: 2519-30, 1999). The cells were transformed with a series of N_{ub}-FPR1-C_{ub}—RURA3 and N_{ub}-FPR1MC—C_{ub}—RURA3 constructs and with a series of N_{ub}-GUK1-C_{ub}—RURA3 and N_{ub}-GUK1ΔH—C_{ub}—RURA3 constructs. The growth of the transformed cells on SD-ura reflects the different arrangements of the N- and the C-termini in the structures of Guk1p and Fpr1p (FIG. 6b). Nᵢₐ-Fpr1-C_{ub}—RUra3p already supports the growth of the cells whereas the cells require the N_{ag}-Guk1-C_{ub}—RUra3p to achieve the same phenotype. N_{ag} has a much lower affinity for C_{ub} than Nᵢₐ. Fpr1MC, one of the mutants of Fpr1p, can be distinguished from the wild type protein by comparing the growth of the cells bearing the Nᵢₐ at the N-terminus of both fusion constructs. Growth is solid for the cells harboring the native Nᵢₐ—FPR1-C_{ub}—RURA3, whereas growth is very poor for cells containing Nᵢₐ—PPR1MC—C_{ub}—RURA3 (FIG. 6b). This assay therefore confirms the more unfolded nature of Fpr1MC and links the structural stability of the molecule to a selectable phenotype. The same assay also reveals a destabilizing mutation in Guk1p. Cells bearing the N_{vg}-construct of GUK1ΔH grow, whereas the cells containing the corresponding construct of the wild type GUK1 grow only poorly on SD-ura (FIG. 6b). Interestingly, the growth assay detects the effect of deleting the helix on the structure of Guk1p more clearly than the immunodetection of the cleaved and uncleaved Dha fusion proteins does (compare FIGS. 5b and 6b).

### Example 1.8

Detecting Conformational Alteration in Sec62-1p

In a first application of this technique, we turned to a commonly encountered problem in molecular genetics. How does a certain mutation influence the activity of the associated gene product? Also, how does an alteration in an environmental factor such as temperature in influencing protein confirmation. We chose a well known allele of SEC62 as our first example. Sec62p is a component of the protein translocation machinery in the membrane of the endoplasmic reticulum of the yeast Saccharomyces cerevisiae. The identification of the gene and its participation in translocation was aided by the discovery of the temperature sensitive sec62-1 allele (Deshaies et al., J. Cell. Biol. 109: 2653-64, 1989; Deshaies et al., Mol. Cell. Biol. 10: 6024-35, 1990). The mutation was recently shown to exchange the glycine at position 46 of Sec62p for aspartate. This mutation occurs in the cytosolic N-terminal domain of the protein (ΔC125; residues 1-158) which is responsible for interacting with the cytosolic C-terminal tail of Sec63p, a further component of the translocation apparatus (Wittke et al., Mol. Biol. Cell 11: 3859-71, 2000). Since the mutation interferes with the tight binding between the two proteins, it was interesting to learn whether this residue was part of the binding interface or influenced the structure or stability of this domain. The structure of this domain is yet unsolved. The reading frames of the N-terminal domain of SEC62, the N-terminal domain of sec62-1 and the domain of a newly constructed allele of SEC62, sec62-1 were inserted between N_{vg} and C_{ub}-Dha. sec62-141 harbors an additional leucine-serine exchange at position 141 beside the mutation at position 46. Sec62p from other species always harbor a large hydrophobic residue at the equivalent of this position. We therefore expected that the hydrophilic serine might cause a destabilization of the protein that can serve as an internal control for the effect of the exchange at position 46.

The cleavage of the three different N_{vg}—C_{ub}-Dha constructs was visualized after protein extraction and immunoblotting with an anti-ha antibody (FIG. 7). We observe a clear shift to the uncleaved fraction of those N_{vg}—C_{ub}-Dha fusion proteins which carry the mutation at position 46 or both 46 and 141 of ΔC125. Whereas hardly any uncleaved fusion construct is detected for the wild type protein, the mutations significantly increase the fraction of uncleaved protein although the sum of cleaved and uncleaved protein is more than twofold smaller for the two mutants (FIG. 7a). The effect is comparable for the two mutants of Sec62p. We could exclude inter- rather than intramolecular reassociation as the cause of the observed cleavage by expressing N_{ig}-ΔC125-Dha and ΔC125-C_{ub}-Dha as separate polypeptides in the same cell (FIG. 7b). Virtually no cleavage was detected although the N_{ig} employed in this assay has a stronger affinity for C_{ub} than the N_{vg} used in the constructs carrying both Ub-peptides at the opposite termini of the same molecule. We therefore conclude that the N-terminal domains of sec62-141p and sec62-1p differ in their conformation or stability from the wild type structure.

### Example 1.9

Materials and Methods

Construction of Fusion Proteins

N_{ub} derivatives containing mutations at position 3 were obtained via PCR using oligonucleotides carrying the corresponding nucleotide exchanges at position 3 and the plasmids carrying the UB4 genes coding for either an I, A, V or G in codon position 13 of the ORF as a template (Johnsson et al., Proc. Natl. Acad. Sci. USA 91: 10340-44, 1994). Fragments encoding the complete ORF of GUK1 (558 bp) or FPR1 (339 bp) were obtained by PCR using yeast genomic DNA (JD53) as a template and an oligonucleotide primer complementary to the 5′ and 3′ ends of the gene, respectively. Fragments containing the ORF of SUMO1 lacking the last 18 nucleotides were obtained via PCR using the plasmid pGEX-SUMO-1 as a template (Bayer et al., J. Mol. Biol. 280: 275-86, 1998). All 5′-primers contained an additional BamHI site and all 3′-primers an additional SalI site to allow for the in-frame fusion with the N_{ub} and C_{ub} moieties. The obtained PCR-fragments were cut with BamHI and SalI and introduced into the correspondingly cut P_{CUP1}—N_{ub}—C_{ub}-Dha cassette on a pR314 vector. The corresponding C_{ub}—RURA3 constructs were obtained by inserting the EagI-SalI cut P_{CUP1}—N_{ub}—ORF in front of the C_{ub}—RURA3 module on a pRS313 vector (Wittke et al., Mol. Biol. Cell 10: 2519-30, 1999). N_{ub}-GUK1-ha, N_{ub}-FPR1-ha, GUK1-C_{ub}-Dha and FPR1-C_{ub}-Dha were derived from the corresponding P_{CUP1}—N_{ub}—X—C_{ub}-Dha constructs. The constructs containing mutations or deletions of the ORFs of GUK1, FPR1 and SUMO1 were obtained via PCR using the corresponding wild type fragments as a template and a combination of primers introducing the desired mutation and the BamHI or SalI site at the 5′ or 3′ end, respectively. GUK1ΔH was obtained by an in frame deletion of the internal ecru fragment (position 172-198).

To obtain the different ΔC125 constructs, a PCR of the first 474 Bp of the ORF of SEC62 was performed using genomic DNA of the yeast strain RSY529 carrying the sec62-1 allele as a template or the yeast strain JD53 carrying SEC62 as a template. The product was digested with SalI and BamHI and inserted into the correspondingly cut vector-cassettes to obtain N_{vg}-ΔC125-C_{ub}-Dha, N_{ub}-ΔC125-Dha and ΔC125-C_{ub}-Dha on the pRS314 and pRS315 vectors, respectively (Wittke et al., Mol. Biol Cell 10: 2519-30, 1999). The constructs of sec62-141 were obtained by chance by a PCR on genomic DNA of RSY529 which introduced a T to C transition at position 422 of the ORF of sec62-1.

More detailed information on the constructs and their generation is available upon request. DNA sequences were determined by the MPIZ DNA facility on PE Biosystems Abi Prism 377 and 3700 sequencers using BigDye-terminator chemistry. Oligonucleotides were purchased from Metabion (Martinsried, Germany) and MWG Biotech (Ebersberg, Germany).

Pulse-Chase Analysis

S. cerevisiae cells expressing N_{vg}—FPR1—C_{ub}-Dha were grown at 30° C. in 10 ml of SD-trp medium to an OD₆₀₀ of ˜1, and supplemented with 100 μM CuSO₄ 30 min prior to labeling the cells for 5 min with Redivue Promix-[³⁵S] (Amersham, Buckinghamshire, UK). The chase, preparation of cell extracts in the presence of N-ethylmaleimide and immunoprecipitation with the monoclonal anti-HA antibody were carried out essentially as described (Johnsson et al., Embo J. 13: 2686-98, 1994). Gels were fixed and the dried gels were exposed and scanned using a PhosphorImager (Molecular Dynamics, Sunnyvale, Calif.).

Immunoblotting

S. cerevisiae cells expressing the different N_{ub}—X—C_{ub}-fusions were grown at 30° C. in 10 nm of SD medium to an OD₆₀₀ of 0.8 and supplemented with 100 μM CuSO₄ one hour prior to cell extraction. Cell extraction for immunoblotting was performed essentially as previously described (Johnsson et al., Embo J. 13: 2686-98, 1994). Proteins were fractionated by SDS-12.5% PAGE and electroblotted onto nitrocellulose membranes (Schleicher and Schuell, Dassel, Germany), using a semi-dry transfer system (Hoefer, Pharmacia Biotech INC., San Francisco, Calif.). Blots were incubated with a monoclonal anti-ha antibody (Babco, Richmond, Calif.). Bound antibody was visualized with horseradish peroxidase-coupled rabbit anti-mouse or goat anti-rabbit antibody (BioRad, Hercules, Calif.), using the chemiluminescence detection system (Pierce, Rockford, Ill.). The chemiluminescence was quantified with the aid of the lumi-imager system (Boehringer, Mannheim, Germany).

Yeast Strains, Growth and Functionality Assays

S. cerevisiae strains were JD53 (MATα his3-Δ200 leu2-3,112 lys2-801 trp 1 Δ63 ura3-52), JD47-13C (MATa of JD53), AG215 (MATα GAL10 GUK1:LEU2 his3) and YDF5 (MATa trp1-901 ade2-101 ura 3-52 leu2-3-112 lys2-801 his3-200 gal4Δgal80ΔLYS2::GAL2-HIS3 URA3::GAL1-lacZ FPR1::ADE2), and RSY529 (MATα his4 leu2-3,112 ura3-52 sec62-1). Yeast rich (YPD) and synthetic minimal media with 2% dextrose (SD) or 2% galactose (SG) followed standard recipes.

Growth assays: S. cerevisiae cells were first grown at 30° C. in liquid selective media containing uracil. Cells were diluted in water and dilutions were spotted on agar plates selecting for the presence of the fusion constructs and lacking uracil. The same dilutions were spotted onto plates containing uracil to check for cell numbers. The plates were incubated at 30° C. for 2-3 days.

Functionality assays: The strain AG215 expressing GUK1 under the control of the P_{GAL10}-promoter was mated with JD47-13c and a resulting diploid was sporulated and tetrades were dissected on yeast rich medium containing galactose. Spores were selected for the right marker combination and the obtained strain was transformed with the TRP1-plasmids containing the different GUK1 fusion constructs and grown on SG-trp,-leu washed in water and spotted onto SD-trp,-Ieu plates to suppress the expression of the P_{GAL1} controlled GUK1. Restoration of growth under these conditions indicated the functionality of the constructs (Konrad, J. Biol. Chem. 267: 25652-55, 1992). The strain YDF5 containing a deletion of FPR1 was transformed with the plasmids containing the different FPR1 fusion constructs. The cells were tested for regaining rapamycin sensitivity by a halo assay. Filter disks containing 5 μl of rapamycin (Sigma, Deisenhofen, Germany) at a concentration of either 0.1 or 1 μg/ml were mounted onto media lacking tryptophan to select for the presence of the constructs. Cells were grown at 30° C. for two days. A halo of non dividing cells around the filter disk indicated the functionality of the constructs (Heitman et al., Science 253: 905-09, 1991).

### Example 2

Introduction

Interaction induced changes in the conformation of proteins are frequently the molecular basis for the modulation of their activities. Although proteins perform their functions in cells, surrounded by many potential interaction partners, the studies of their conformational changes have been mainly restricted to in vitro studies. Ste4p (Gβ) and Ste18p (Gγ) are the subunits of a heterotrimeric G-protein in the yeast Saccharomyces cerevisiae. A split-ubiquitin based conformational sensor was used to detect a major structural rearrangement in Ste18p upon binding to a test compound, in this case to the polypeptide Ste4p. Based on these in vivo results and the solved structure of the mammalian Gβγ, it is shown that Gγ of yeast adopts an equally extended structure, which is only induced upon association with Gβ.

### Example 2.1

Split-Ub Based Approach

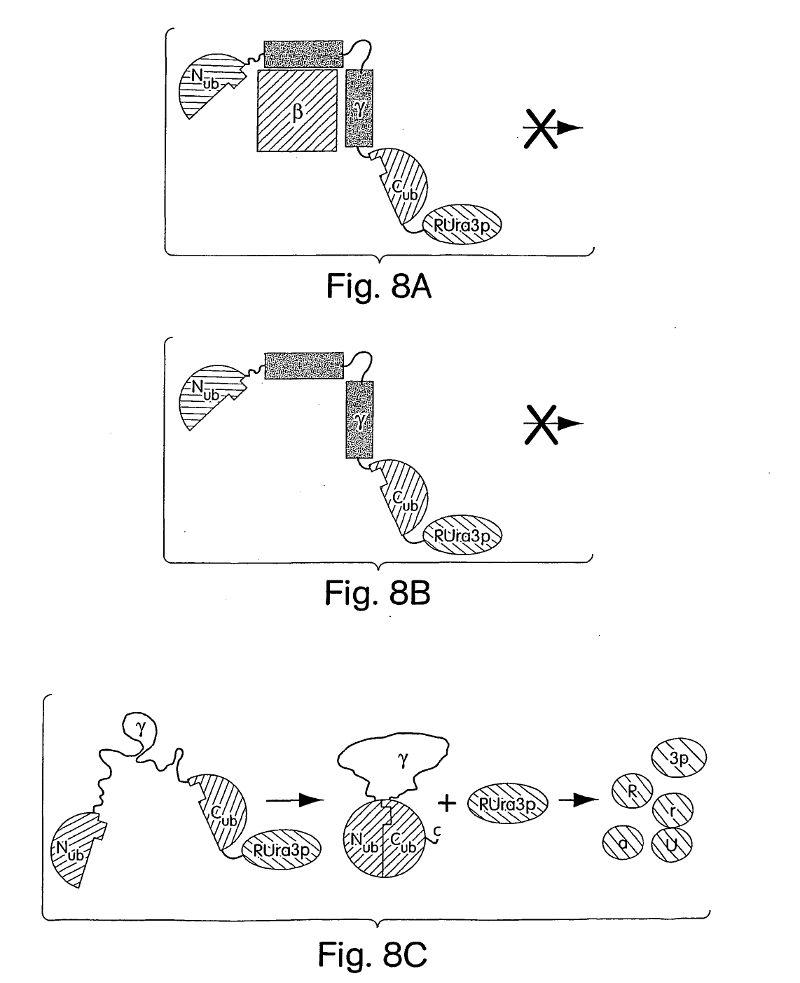

In the proposed structure of the Ste4p-Ste18p complex the N- and the C-termini of Ste18p are spatially separated. This should prevent the association of the N_{ub} and C_{ub} when attached to the N- and the C-termini of the same Ste18p polypeptide. As a result, an RUra3p reporter coupled to the C-terminus of C_{ub} is not cleaved and will enable yeast ura3 cells to grow on plates lacking uracil (FIG. 8A). Two possibilities can be envisioned for the free Ste18p. If the structure of the free protein is indistinguishable from its bound form, the RUra3p reporter will remain linked to C_{ub} and the cells will retain the original phenotype (FIG. 8B). If the conformation of Ste18p is more flexible in its free than in its bound form it will less hinder the N_{ub}—C_{ub} reassociation and the RUra3p reporter will be cleaved off far more efficiently (FIG. 8C). The enzymes of the N end rule pathway rapidly degrade the released RUra3p rendering the cells uracil auxotrophic (Wittke et al., Mol. Biol. Cell 10: 2519-2530, 1999; Varshavsky, Genes Cells 2: 13-28, 1997). The phenotype of cells that express N_{ub}-Ste18-C_{ub} in the absence of Ste4p will thus reflect the conformation of the uncomplexed Gγ.

### Example 2.2

Choosing a N_{ub} Mutant with an Appropriate Affinity for C_{ub}

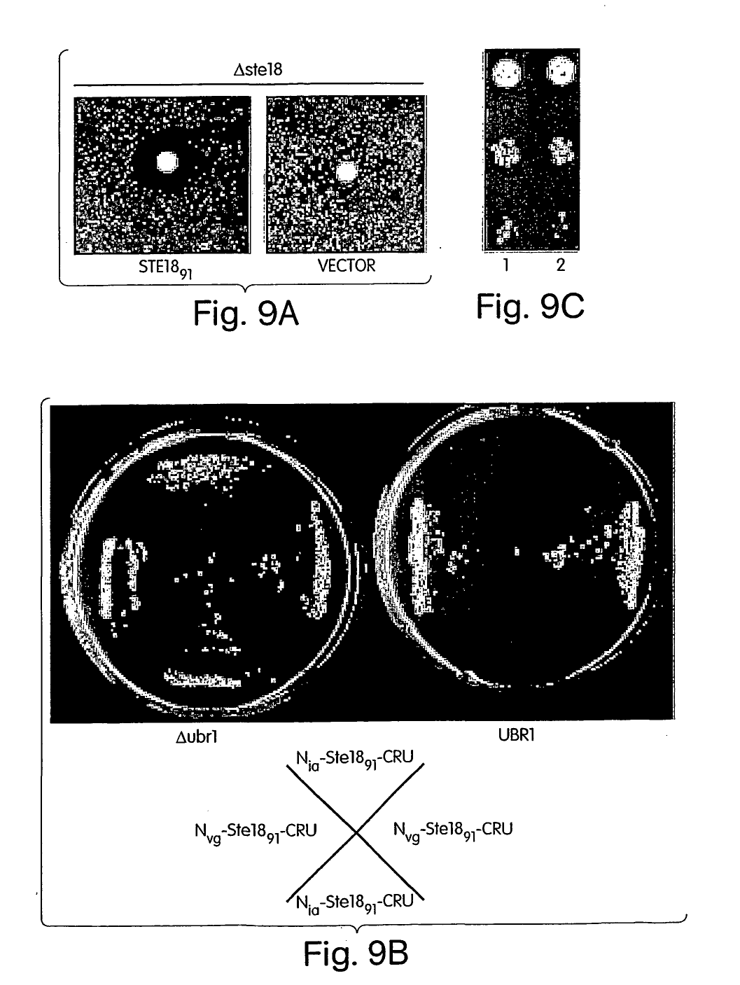

The first 19 residues of Ste18p are unique to Gγ of the yeast. The structure of this N-terminal stretch could therefore not be predicted and its sequence was deleted to create Ste18₉₁p. α-Factor induced growth inhibition of cells expressing Ste1891p instead of the wild-type protein documented an undiminished functionality of Ste1891p and thereby indirectly its binding to Ste4p (FIG. 9A, and data not shown) (Clark et al., Mol. Cell. Biol. 13: 1-8, 1993). A STE18₉₁ construct that lacked the last five C-terminal residues including the motif for isoprenylation was placed between N_{ub} and C_{ub}—RURA3. Our previous work has shown that the affinity between N_{ub} and C_{ub} is critical for reflecting the conformation of a protein in a N_{ub}—C_{ub} fusion (Raquet et al., J. Mol. Biol. 305: 927-938, 2001). A N_{ub} with a too strong affinity for C_{ub} will override the effect of the conformation of the inserted Ste18p whereas a N_{ub} with a too low affinity for C_{ub} will not sense any alterations in the conformation. Since we predicted that the conformation of the uncomplexed Ste18p would favor rather than impede the N_{ub}—C_{ub} reassociation we first had to choose a N_{ub} that just inhibits the growth of the N_{ub}-Ste18₉₁-C_{ub}—RUra3p transformed cells. Nᵢᵢ and Nᵢₐ in a N_{ub}-STE18₉₁-C_{ub}—RURA3 construct both inhibit the SD-ura growth of cells, which do not overexpress Ste4p (FIG. 9B, and data not shown). We chose Nᵢₐ for our further studies since Nᵢₐ has a weaker affinity for C_{ub} and should therefore react more sensitively to alterations in the conformation of Ste18p. We performed two control experiments to show that the uracil auxotrophy of the cells reflects the efficient Nᵢₐ—C_{ub} reassociation within the Nᵢₐ-Ste18₉₁-C_{ub}—RUra3p and not a rapid degradation of the uncleaved fusion protein (Clark et al., Mol. Cell. Biol. 13:1-8, 1993; Hirschman et al., J. Biol. Chem. 272: 240-248, 1997). N_{vg} displays a lower affinity for C_{ub} that results in the accumulation of a larger fraction of uncleaved fusion protein than observed with the otherwise identical Nᵢₐ construct. The growth of cells on SD-ura proves that the uncleaved N_{vg}-Ste18₉₁-C_{ub}—RUra3p is sufficiently stable (FIG. 9B). This interpretation was further corroborated by the growth of the ubr1 cells expressing Nᵢₐ-Ste18₉₁-C_{ub}—RUra3p on SD-ura (FIG. 9B). This strain lacks a functional N end rule and will not degrade the cleaved RUra3p (Wittke et al., Mol. Biol. Cell 10: 2519-2530, 1997; Varshavsky, Genes Cells 2: 13-28, 1997). We conclude that the efficient cleavage at the C-terminus of C_{ub} followed by the rapid degradation of the released RUra3p causes uracil auxotrophy of the corresponding isogenic wild-type cells. An intramolecular or an intermolecular Nᵢₐ—C_{ub} reassociation could induce this efficient cleavage. The latter reaction might reflect the propensity of the free Ste18p to form defined complexes or aggregates. To distinguish between these two alternatives we coexpressed N_{vg}-Ste18₉₁-C_{ub}—RUra3p together with a Nᵢₐ-STE18₉₁ construct which was C-terminally extended by Dha to facilitate the detection of the protein by immunoblotting (Nᵢₐ-Ste18₉₁-Dha). If the free Ste18p forms dimers or multimers, the coexpression of Nᵢₐ-Ste18₉₁-Dha should increase the cleavage of N_{vg}-Ste18₉₁-C_{ub}—RUra3p and thereby inhibit the SD-ura growth of the cells. This was not observed. The cells coexpressing N_{vg}-Ste18₉₁-C_{ub}—RUra3p and Nᵢₐ-Ste18₉₁-Dha still grow nearly as well on SD ura as the cells expressing N_{vg}-Ste18₉₁-C_{ub}—RUra3p alone, although Nᵢₐ-Ste18₉₁-Dha was clearly detected in extracts of the cells by the anti-HA antibody (FIG. 9C, and data not shown).

### Example 2.3

Ste18p Undergoes a Change in Conformation upon Binding to Ste4p

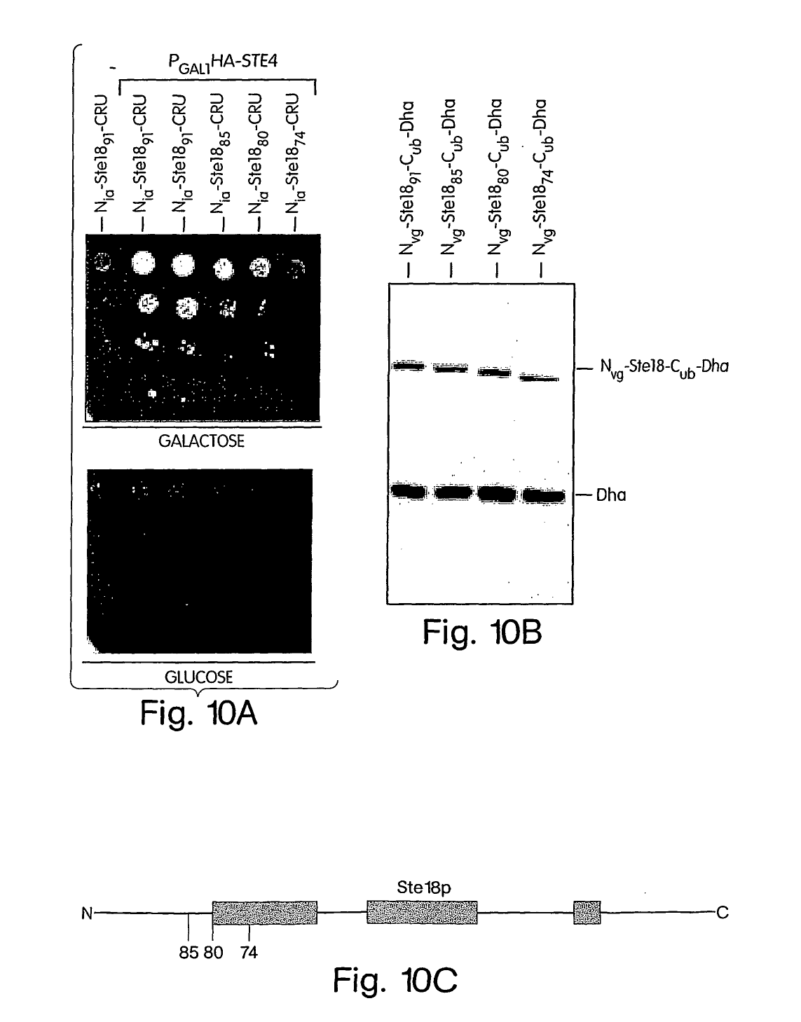

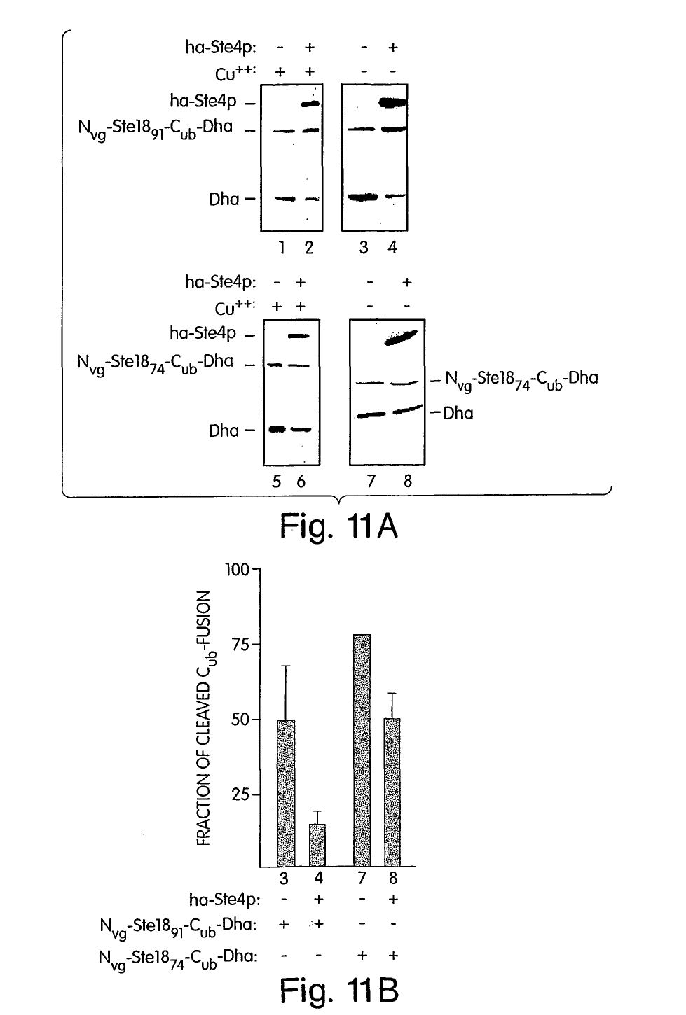

To measure the influence of Ste4p on the conformation of Ste18p, we coexpressed Nᵢₐ-Ste18₉₁-C_{ub}—RUra3p together with HA-tagged Ste4p (HA-Ste4p) in cells lacking the chromosomal STE18 gene. The cells were spotted in different dilutions onto plates without uracil and containing either glucose or galactose as the carbon source. Since the expression of HA-STE4 was controlled by the P_{GAL1} promoter, the intracellular HA-Ste4p concentration was high on galactose medium but below the limits of detection on glucose medium (FIG. 11A, and data not shown). Transformants containing these constructs were uracil auxotrophs on glucose medium, but did grow without uracil on medium containing galactose (FIG. 10A). This effect was due to the presence of the HA-STE4 expressing plasmid since cells containing an empty vector instead were unable to grow without uracil independent of the nature of the carbon source (FIG. 10A). The outcome of this analysis supports the model that upon binding to Ste4p, Ste18p undergoes a substantial conformational change that interferes with the efficient interaction between the coupled N_{ub} and C_{ub} (FIG. 8 and FIG. 10). To further confirm the predicted structure of the Ste4p bound Ste18p, we performed the identical assays with STE18 constructs in which an increasing portion of the N-terminus was removed from Ste18₉₁p. The deletion of another six or 11 residues in the STE18₈₅ and STE18₈₀ constructs already diminished the effect of the expression of HA-Ste4p on the SG-ura growth of those cells (FIG. 10A). Removing a further five residues form the N-terminus (Ste18₇₄), and thereby invading the predicted N-terminal helix which constitutes a part of the central binding interface, completely inhibits the SG-ura growth of the construct transformed cells (FIG. 10A). To compare the expression levels of the different fusion proteins, we replaced the RUra3p reporter with the Dha moiety and exchanged the Nᵢₐ against the N_{vg} to create the corresponding N_{vg}-Ste18-C_{ub}-Dha constructs. Since the released Dha bears a stabilizing N-terminus, we could simultaneously detect the cleaved and the uncleaved fusion-proteins. The immunoanalysis revealed comparable expression levels for all fusion proteins (FIG. 10B). We therefore conclude that the N-terminal deletions of Ste18p reduce the binding to Ste4p (FIG. 10A).

### Example 2.4

Quantitative Analysis of Conformation Change Using the Split Ubiquitin System

We used the Dha reporter constructs to perform a more quantitative analysis by protein extraction and immunoblotting. N_{vg}-Ste18₉₁-C_{ub}-Dha or N_{vg}-Ste18₇₄-C_{ub}-Dha was either expressed alone or together with HA-Ste4p (FIG. 11). Expression of the N_{vg}-Ste18-C_{ub}-Dha constructs from the P_{CUP1} promoter was held at a relatively low level or was induced by addition of copper ions to 100 μM 1 hr prior to protein extraction. The ratio of uncleaved to cleaved fusion protein was calculated after denaturing gel electrophoresis and immunoblotting with the anti-HA antibody. This value was compared to the ratio of uncleaved to cleaved N_{vg}-Ste18-C_{ub}-Dha from protein extracts of cells lacking HA-Ste4p. The expression of HA-Ste4p increases the fraction of uncleaved N_{vg}-Ste18₉₁-C_{ub}-Dha by a factor of two under inducing conditions (+Cu²⁺) and a factor of 3.6 under non-inducing conditions (FIGS. 11A, B). These experiments confirm the results of the growth assays that Ste18p undergoes a measurable change conformation upon binding to Ste4p. No increase in the uncleaved fraction was observed for the N_{vg}-Ste18₇₄-C_{ub}Dha under inducing conditions (+Cu²⁺) (FIG. 11A). However, a 1.6-fold increase in the ratio of uncleaved to cleaved fusion protein was detected when the expression of N_{vg}-Ste18₇₄-C_{ub}-Dha was not induced by the addition of copper before extraction (FIGS. 11A, C). This relatively small effect might reflect a weak binding activity of Ste18₇₄p to Ste4p, which is not detected by the less sensitive growth assay (FIG. 10A). This interpretation is supported by experiments showing that similarly truncated γ-subunits display a still detectable albeit much reduced affinity for their cognate β-subunits (Mendeet al., J. Biol. Chem. 270: 15892-15898, 1995; Mason and Botella, Proc. Natl. Acad. Sci. USA 97: 14784-14788, 2000).

There could be several reasons for employing such a significant conformational transformation in Gγ upon binding Gβ. Since the unfolded Gγ seems to exist only briefly before and during the maturation of Gβγ, it is an unlikely, even though not disproved possibility that the unfolded state of Ste18p fulfills additional roles besides serving as a precursor for Gβγ (Rehm and Ploegh, J. Cell Biol. 137: 305-317, 1997). Alternatively, the lack of structure might be a mechanism to protect against unproductive or unwanted protein associations. By inducing the fold of Ste18p and thereby creating the binding interface for other proteins, Ste4p ensures that any interactions with Ste18p will only occur within the Ste18p-Ste4p complex. The subset of interactions that is detectable between other components of the signaling cascade and Gβγ and not with its separated subunits might fall into this category (Whiteway et al., Science 269: 1572-1575, 1995; Pryciak and Hartwell, Mol. Cell. Biol. 16:2614-2626, 1996; Leeuw et al., Nature 391: 191-195, 1998).

### Example 2.5

Materials and Methods

Construction of Fusion Proteins

Fragments containing the open reading frame (ORF) of STE18 lacking either the first 57 (STE18₉₁), 75 (STE18₈₅), 90 (STE18₈₀), or 108 (STE18₇₄) nucleotide and lacking the last 15 nucleotides were obtained by PCR using yeast genomic DNA, the Pwo polymerase (Roche-Biochemicals, Penzberg, Germany) and oligonucleotide primers complementary to the 5′ and 3′ ends of the desired ORF, respectively (Metabion, Martinsried, Germany). All 5′ primers contained an additional BamHI site, the 3′ primer an additional SalI site to allow for the in-frame fusion with the N_{ub} and C_{ub} moieties (Raquet et al., J. Mol. Biol. 305: 927-938, 2001). The border between N_{ub} and the different STE18 constructs reads GGG ATC CCC XXX, where =is CAG as the first codon for STE18₉₁, AAG for STE18₈₅ and STE18₈₀, and GAA for STE18₇₄. The corresponding C_{ub}—RURA3 constructs were obtained by inserting the EagI-SalI cut P_{CUP1}—N_{ub} ORF in front of the C_{ub}—RURA3 module on a pRS313 vector (Wittke et al., Mol. Biol. Cell 10: 2519-2530, 1999). Nᵢₐ-STE18₉₁-Dha was derived from the corresponding P_{CUP1}—N_{ub}—STE18₉₁-C_{ub}-Dha constructs by cloning the EagI-SalI fragment in front of the ORF of DHFR extended by the HA epitope (Dha). STE18₉₁ containing the natural stop codon and an additional start codon at the 5′ end was obtained by PCR using an oligonucleotide complementary to the 3′ region starting 61 bp downstream of the ORF and an oligonucleotide complementary to the 5′ region of the ORF. The HA-STE4 construct was obtained by PCR using an oligonucleotide complementary to a 3′ region stating 60 bp downstream of the ORF and an oligonucleotide complementary to the 5′ end of the ORF. The introduced SalI and KpnI sites were used to clone the PCR fragment downstream and in-frame of the P_{GAL1}-HA module in a pRS416 vector. The 5′ sequence of the newly generated ORF reads: ATG TCG ACC TAC CCA TAC GAT GTT CCA GAT TAC GCT GGC TCG ACC ATG (SEQ ID No. 3). The sequence of the HA epitope is underlined. The first codon of STE4 is printed in bold letters.

Immunoblotting

S. cerevisiae cells expressing the different fusion proteins were grown at 30° C. to an OD₆₀₀ of ˜0.8 in 10 ml of SG lacking tryptophan and uracil. Cell extraction for immunoblotting was performed essentially as previously described (Johnsson and Varshavsky, EMBO J. 13: 2686-2698, 1994). Bound antibody was visualized with horseradish peroxidase coupled rabbit anti-mouse antibodies (Bio-Rad, Hercules, Calif., USA), using the chemiluminescence detection system (Pierce, Rockford, Ill., USA) and quantified with the lumi-imager system (Boehringer, Mannheim, Germany).

Yeast Strains, Functionality Assay

S. cerevisiae starins were JD53 (MATα his3-Δ200 leu2-3,112 lys2-801 trp1-Δ63 ura3-52), JD55 (MATα his3-Δ200 leu2-3,112 lys2-801 trp1-63 ura 3-52 ubr1::HIS3), KMY940 (MATa his3-11,15 leu2-3,112 ade2-1 trp1-1 ura3-1 can1-100 ste18::LEU2), YEL2 (MATa his3-11,15 leu2-3,112 ade2-1 trp1-1 ura3-1 can1-100ste4::URA3). Yeast rich and synthetic minimal media with 2% dextrose (SD) or 2% galactose (SG) followed standard recipes.

In the functionality assay, the cells were tested for α-factor sensitivity by a halo assay. Filter disks containing 2.4 μg of α-factor were mounted onto media lacking tryptophan or uracil to select for the presence of the plasmids expressing the constructs and 2% galactose to express HA-Ste4p. Cells were grown at 30° C. for 1 day.

### Example 3

Detecting Conformation Change in p53

The tumor suppressor gene p53 has been identified as the most frequent target of genetic alterations in human cancers. Most of these mutations occur in highly conserved regions in the DNA-binding core domain of the p53 protein, suggesting that the amino acid residues in these regions are critical for maintaining normal p53 structure and function.

Using molecular dynamics calculations, Chen et al. (J. Protein Chem. 20: 101-5, 2001) demonstrate that several amino acid substitutions (namely His 175, Asp 245, Asn 245, Trp 248, Met 249, Ser 278, and Lys 286) in these regions that are induced by environmental carcinogens and found in human tumors produce certain common conformational changes in the mutant proteins that differ substantially from the wild-type structure. The results indicate that all of these mutants differ substantially from the wild-type structure in certain discrete regions and that some of these conformational changes are similar for these mutants as well as those determined previously. The changes are also consistent with experimental evidence for alterations in structure in p53 mutants determined by epitope detectability using monoclonal antibodies directed against these regions of predicted conformational change.

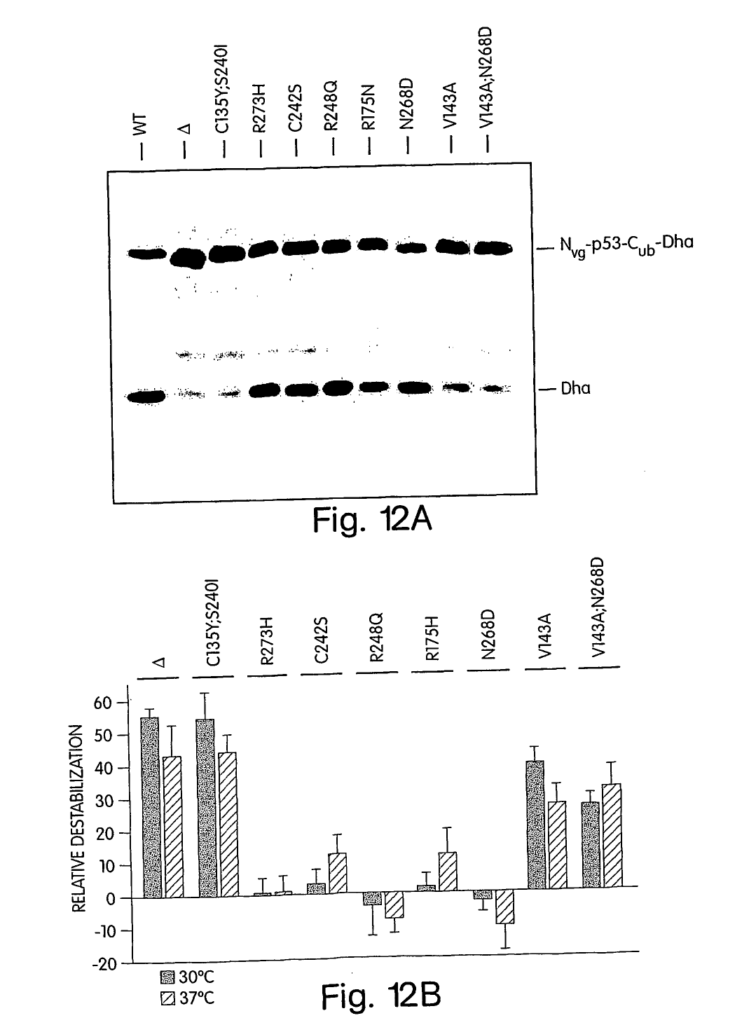

In order to investigate the use of the split-ubiquitin system of the instant invention in determining mutant p53 conformation change, nine p53 core domain (amino acids 102-292 of 394 in total) missense point mutants or double point mutants C135Y; S240I, R273H, C242S, R248Q, R175H, N268D, V143A, V143A; N268D , and deletion mutants Δ (missing a stretch of amino acids of the first beta-strand of the p53core), as well as wild-type were cloned between N_{vg} (I3V, I13G) and C_{ub}-Dha (HA-tagged DHFR as reporter for western blot analysis) to generate the N_{vg}-p53_{core}-C_{ub}-Dha fusion protein constructs. To measure the effects of these cancer causing mutations on the stability of the p53core in vivo, N_{vg}-p53core-C_{ub}-Dha and the nine different p53 core mutants were expressed in yeast at 37° C. and the cleaved and uncleaved fraction of the fusion proteins were probed with the anti-HA antibody on a nitrocellulose blot after cell extraction and SDS-PAGE. The amount of cleaved and uncleaved fusion protein were quantified by chemiluminescence. The amino acid exchanges of the different p53 core mutants are indicated at the top of the blot (FIG. 12A). Representation of the effect of different mutations on the stability of the p53core was shown in FIG. 12B. For each experiment the ratio of uncleaved N_{vg}—P53core-C_{ub}-Dha was calculated. This value was subtracted from the ratio of uncleaved N_{vg}—P53core-C_{ub}-Dha of the different mutants of p53core. A positive value, indicating a higher fraction of uncleaved fusion protein, indicates destabilization of the mutant protein whereas a negative value indicating a higher fraction of cleaved Dha indicates stabilization of the p53core. Experiments were performed at 30° C. (black bars) and 37° C. (grey bars). Five independent experiments at each temperature are shown except for C242S (four experiments at 37° C.) and R175H (six experiments at 37° C.). The efficiency of cleavage was determined by Western blot analysis using an anti-HA antibody to detect cleaved and uncleaved Dha as described before (see above). With all mutations that were expected to reduce stability of the core domain, a positive readout was detectable by this analysis. It is apparent that several mutants with mutations in the DNA binding domain reacted similar to wild-type p53 in this assay, while some other core domain mutants (for example, the V143A single and double mutants) destabilizes the core domain and are likely to have a different conformation.

The wild-type core domain and a destabilizing mutation V143A were further tested using a simple growth assay employing the R-ura3 reporter (see above) and selecting clones that can grow on 5-FOA. In this case the N_{ub} (I13A) mutant Nᵢₐ was used. If the N_{ub}—X—C_{ub}—R-ura3 fusion is stable, the R-ura3 reporter will be cleaved off of the C-terminal end of C_{ub} and subsequently be degraded by N-end rule components (see above), allowing the host yeast cell to grow on the 5-FOA selective media. The less stable the p53 mutant, the less cleavage occurs, and thus the less growth of the host cell is observed.

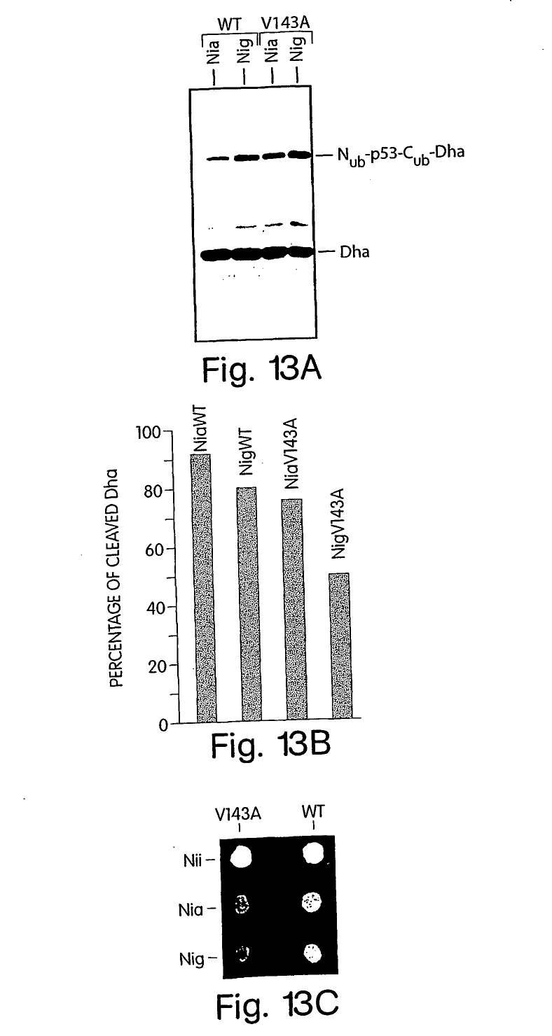

FIG. 13A shows a Western bot of protein extracts of yeast cells expressing N_{ub}-p53core-C_{ub}-Dha and N_{ub}-V143A-C_{ub}-Dha containing the N_{ub}-mutants Nᵢₐ and N_{ig}. The quantification of the experiment is shown in FIG. 13B. Consistent with previous results, the V143A core mutant is a destabilizing mutant when compared to the wild-type p53. FIG. 13C shows a growth assay of yeast cells expressing the corresponding Nᵢᵢ, Nᵢₐ, and N_{ig} N_{ub}-p53core-C_{ub}—RUra3p fusion proteins on media containing 5-FOA. While cells harboring the wild-type core domain did grow under the selected condition, the mutation in the core domain (V143A) significantly reduced growth of the cells. The Nᵢₐ fusion protein allows to distinguish between the cells expressing the wild type p53_{core} (growth) and the cells expressing the V143A mutant of the core (non-growth).

Therefore, the instant invention provides an assay to identify substances/compounds that can stabilize p53-V143A (or any of the other destabilizing mutants of p53 or other proteins). High throughput screen assay can be set up to screen for test compounds (such as small molecules, chemical compounds, etc.) that can stabilize those unstable mutants, thereby correcting defects of mutant proteins. This can be particularly useful in treating cancer and a variety of other disease, wherein restoration of wild-type p53 function can trigger apoptosis in cancer cells and/or induce growth arrest of abberantly proliferating cells.

Although the test shown in this example is carried out in yeast cells, the same assay and also other methods of the invention can also be carried out in mammalian cells with minor modifications using mammalian selectable markers (see above) which are apparent to a skilled artisan. Similarly, using an appropriate mixture of recombinant fusion proteins of the invention and for example, a ubiquitin-specific protease, the method of the invention may be practiced in a cell-free environment.

Equivalents

Those skilled in the art will recognize, or be able to ascertain using no more than routine experimentation, many equivalents of the specific embodiments of the invention described herein. Such equivalents are intended to be encompassed by the following claims.

1. A fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide selected from the group consisting of: Guk1, Fpr1, Sec62p, beta-amyloid, p53, calmodulin, estrogen receptor alpha (ERα), FKBP, G-protein, VHL, tyro sine kinases, Src, Abl, Epidermal Growth Factor receptor (EGFR), Protein Kinase A (PKA), Protein Kinase C (PKC), Cyclophillins, Cyclin Dependent Kinases (CDKs), Cyclins, a protein of therapeutic, physiological or biological interest or variants/fragments thereof.

2. A fusion protein comprising the structure N_{ub}—X—C_{ub}—RM, wherein C_{ub} is a carboxy-terminal ubiquitin domain, RM is a reporter moiety fused to the carboxy-terminus of the C_{ub} domain, X is a nonubiquitin polypeptide, and N_{ub} is a mutant amino-terminal ubiquitin domain selected from the group consisting of: Nᵥᵢ, Nᵥₐ, N_{vg}, Nₐᵢ, Nₐₐ, N_{ag}, N_{gi}, N_{ga}, and N_{gg}.

3. A fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a nonubiquitin polypeptide, wherein RM is a selectable marker.

4. A fusion protein comprising the structure N_{ub}—X—C_{ub}—RM, wherein C_{ub} is a carboxy-terminal ubiquitin domain, RM is a reporter moiety fused to the carboxy-terminus of the C_{ub} domain, X is a nonubiquitin polypeptide, and N_{ub} is a mutant amino-terminal ubiquitin domain which has altered affinity for C_{ub} chosen such that for a given X polypeptide it just inhibits or just allows the reconstitution of a quasi-native ubiquitin and hence cleavage of RM from the fusion protein.

5. A fusion protein comprising the structure N_{ub}—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain, and X is a non-yeast nonubiquitin polypeptide.

6. A fusion protein comprising the structure N_{ub}—X—C_{ub}—RM, wherein N_{ub} is an amino terminal ubiquitin domain or a mutant amino-terminal ubiquitin domain, C_{ub} is a carboxy-terminal ubiquitin domain, X is a nonubiquitin polypeptide, and RM is a reporter moiety fused to the carboxy-terminus of the C_{ub} domain, wherein upon cleavage of the C_{ub}—RM junction, the first amino acid of the released RM is an amino acid other than methionine.

7. The fusion protein of claim 6, wherein the first amino acid of the cleaved RM is Arginine, Lysine, Histidine, Phenylalanine, Tryptophan, Tyrosine, Leucine, Aspartate, Glutamate, Cysteine, Asparagine, Glutamine or Isoleucine.

8. A polynucleotide sequence encoding any one of the fusion proteins of claims 1-6.

9. A host cell harboring a polynucleotide sequence encoding any one of the fusion proteins of claims 1-6.

10. A method of detecting a conformational change in a polypeptide resulting from a mutational alteration in the polypeptide sequence comprising:
1) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety, wherein upon cleavage of the C_{ub}—RM junction, the first amino acid of the released RM is an amino acid other than methionine; and,
2) measuring a second fusion protein reporter moiety activity from a N_{ub}—X′—C_{ub}—RM, wherein X′ is a mutationally altered form of polypeptide X;
wherein a change in the level of the second fusion protein RM activity relative to the first fusion protein RM activity indicates that the polypeptide has undergone a conformation change resulting from the mutational alteration.

11. A method of detecting a conformational change in a polypeptide resulting from a point mutation or an insertion/deletion of no more than 3 amino acids in the polypeptide sequence comprising:
1) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reporter moiety; and,
2) measuring a second fusion protein reporter moiety activity from a N_{ub}—X′—C_{ub}—RM, wherein X′ is a point mutation or a deletion/insertion of no more than three amino acids form of polypeptide X;
wherein a change in the level of the second fusion protein RM activity relative to the first fusion protein RM activity indicates that the polypeptide has undergone a conformation change resulting from the mutational alteration.

12. A method of detecting a conformational change in a polypeptide resulting from a stimulus comprising:
1) measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin polypeptide of interest and RM is a reported moiety; and,
2) measuring a second fusion protein reporter moiety activity from a N_{ub}—X′—C_{ub}—RM, wherein X′ is the X polypeptide which has been altered by the stimulus;
wherein a change in the level of the second fusion protein RM activity relative to the first fusion protein RM activity indicates that the polypeptide has undergone a conformation change resulting from the stimulus.

13. The method of claim 12, wherein the stimulus is an alteration in environmental factor.

14. The method of claim 13, wherein the alteration in environmental factor is pH change, temperature change, pressure change, redox-state change or ionic strength change.

15. The method of claim 12, wherein the stimulus is a post-translational modification of the X protein.

16. The method of claim 15, wherein the post-translational modification is phosphorylation, methylation, prenylation, acetylation, palmitoylation, myristoylation, reduction, oxidation, glycosylation, proteolytic cleavage, sulfation, hydroxylation, carboxylation, sumoylation, ubiquitination, or modification by ubiquitin-like proteins.

17. The method of claim 12, wherein the stimulus is contacting the X protein with a test compound in trans.

18. The method of claim 17, wherein the test compound is selected from the group consisting of: a polypeptide, a hormone, a steroid, an ion, a polynucleotide, an oligosaccharide, a lipid, an enzyme substrate, a gas molecule, a small molecule, a co-factor, a vitamin, a metal ion, and a nucleotide phosphate.

19. The method of any one of claims 10-12; wherein the nonubiquitin polypeptide of interest is selected from the group consisting of: Guk1, Fpr1, Sec62p, beta-amyloid, p53, calmodulin, estrogen receptor alpha (ERα), FKBP, and G-protein, VHL, tyrosine kinases, Src, Abl, Epidermal Growth Factor (EGF) receptor, Protein Kinase A (PKA) Protein Kinase C (PKC), Cyclophillins, Cyclin Dependent Kinases (Cdk), Cyclins, a protein of therapeutic, physiological or biological interest or variants/fragments thereof.

20. The method of any one of claims 10-12, wherein the N_{ub} domain is an amino-terminal ubiquitin domain or a mutant amino-terminal ubiquitin domain selected from the group consisting of: Nᵢₐ, N_{ig}, Nᵥᵢ, Nᵥₐ, N_{vg}, Nₐᵢ, Nₐₐ, N_{ag}, N_{gi}, N_{ga}, and N_{gg}.

21. The method of any one of claims 10-12, wherein the reporter moiety (RM) is a selectable marker.

22. The method of any one of claims 11-12, wherein the first amino acid of the RM is a non-methionine residue when the RM is released by cleavage of the C_{ub}—RM junction by a ubiquitin-specific protease (UBP).

23. The method of any one of claims 10-12, wherein N_{ub} is a mutant amino-terminal ubiquitin domain which has altered affinity for C_{ub} chosen such that for a given X polypeptide it just inhibits or just allows the reconstitution of a quasi-native ubiquitin and hence cleavage of RM the fusion protein.

24. The method of any one of claims 10-12, wherein X is a non-yeast nonubiquitin polypeptide.

25. The method of any one of claims 10-12 wherein at least one step is performed in a host cell expressing a ubiquitin-specific protease.

26. A method to identify a compound which can change the conformation of a protein upon contacting the protein, comprising:
1) providing a plurality of test compounds which are not known to be able to cause the conformation change of the protein;
2) testing each compound by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin protein of interest, and RM is a reporter moiety; and, measuring a second fusion protein reporter moiety activity from a N_{ub}—X′—C_{ub}—RM, wherein X′ is the X protein which has been altered by the test compound;
wherein a change in the level of the second fusion protein RM activity relative to the first fusion protein RM activity indicates that the X protein has undergone a conformation change resulting from contacting the test compound, thereby identifying a compound which can change the conformation of the protein.

27. The method of claim 26, further comprising formulating the identified compound into a pharmaceutical composition.

28. The method of claim 26, wherein the plurality of test compounds is a library of compounds which comprises greater than 10 test compounds.

29. A method to identify a mutation in a protein which leads to the conformation change of the protein, comprising:
1) generating a plurality of candidate mutations of the protein;
2) testing each candidate mutation by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is a nonubiquitin protein of interest and RM is a reporter moiety; and, measuring a second fusion protein reporter moiety activity from a N_{ub}—X′—C_{ub}—RM, wherein X′ is a mutational altered form of the X protein harboring the candidate mutation;
wherein a change in the level of the second fusion protein RM activity relative to the first fusion protein RM activity indicates that the X protein has undergone a conformation change resulting from the candidate mutation, thereby identifying a mutation which can change the conformation of the protein.

30. A method to identify a protein which changes conformation upon contacting a given compound or encountering an alteration in environmental factor, comprising:
1) providing a plurality of test proteins;
2) testing each protein X by measuring a first fusion protein reporter moiety activity from a fusion protein comprising the structure N_{ub}—X—C_{ub}—RM, wherein N_{ub} is an amino-terminal ubiquitin domain or mutant amino-terminal ubiquitin domain, C_{ub} is a carboxy-terminal ubiquitin domain, X is the nonubiquitin test protein, and RM is a reporter moiety; and, measuring a second fusion protein reporter moiety activity from a N_{ub}—X′—C_{ub}—RM, wherein X′ is the X protein which has been altered by contacting the given compound or by the given alteration in environmental factor;
wherein a change in the level of the second fusion protein RM activity relative to the first fusion protein RM activity indicates that the X protein has undergone a conformation change resulting from the given alteration in environmental factor or contacting the given compound, thereby identifying a protein which changes conformation upon contacting a given compound or encountering an alteration in environmental factors.

31. A method to conduct a business, comprising:
1) by the method of claim 31, identifying one or more compounds which change the conformation of a polypeptide;
2) conducting therapeutic profiling of said identified compounds, or other derivatives thereof, for using the compounds in therapy for a condition; and,
3) formulating a pharmaceutical preparation including one or more compounds identified in (ii) as a product having an acceptable therapeutic profile.

32. The business method of claim 31, further comprising establishing a distribution system for distributing said product for sale.

33. The business method of claim 31, further including establishing a sales group for marketing the product.

34. A method to conduct a business, comprising:
1) by the method of claim 26, identifying one or more compounds which change the conformation of a polypeptide;
2) conducting therapeutic profiling of said identified compounds, or other derivatives thereof, for using the compounds in therapy for a condition; and,
3) licensing, to a third party, the rights for further development of compounds and/or formulating a pharmaceutical preparation including one or more compounds identified in (ii) to affect conformation change of the polypeptide for treatment of the condition.

35. A method to conduct a business, comprising:
1) by the method of claim 10, 11, 12, 26, 29, or 30, generating information or data, or identifying compounds, proteins or mutations/variants/derivatives thereof;
2) licensing, selling, providing for consideration or access to said information, said data, said identified compounds, proteins or mutations/variants/derivatives thereof.

36. A kit for detecting or identifying alterations in the conformation of an X protein, comprising a panel of at least two vector constructs for expressing fusion proteins of the general structure N_{ub}—X—C_{ub}—RM, wherein each vector construct comprises a coding sequence for N_{ub}, an amino-terminal ubiquitin domain or a mutant amino-terminal ubiquitin domain selected from Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, or N_{gg}; a coding sequence for C_{ub}, a carboxy-terminal ubiquitin domain; a coding sequences for RM, a reporter moiety fused to the carboxy-terminus of the C_{ub} domain; and at least one cloning site or multicloning site for subcloning the X-protein in-frame with both the N-terminal N_{ub} and the C-terminal C_{ub}—RM moieties; and wherein at least one vector construct expresses a mutant N_{ub} fusion protein.

37. The kit of claim 36, further comprising a host cell for expressing said fusion proteins from said vector constructs.

38. The kit of claim 36, further comprising instructions for detecting or identifying alterations in protein conformation by using the vector constructs.

39. A method for detecting a conformation change of a protein resulting from a stimulus, comprising:
1) measuring a first spectrum of fusion protein reporter moiety activity from a first panel of at least two fusion proteins, each different from the other, comprising the general structure N_{ub}—X—C_{ub}—RM, wherein N_{ub} is an amino-terminal ubiquitin domain or mutant amino-terminal ubiquitin domain selected from at least one of Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, and N_{gg}, C_{ub} is a carboxy-terminal ubiquitin domain, X is the protein, and RM is a reporter moiety;
2) measuring a second spectrum of fusion protein reporter moiety activity from a second panel of fusion proteins comprising the general structure N_{ub}—X′—C_{ub}—RM, wherein the second panel of N_{ub} and C_{ub} fusion proteins are the same as the first panel of N_{ub} and C_{ub} fusion proteins, X′ is the X protein resulting from treating the X protein with the stimulus, and RM is a reporter moiety;
3) comparing the first and second spectra of fusion protein reporter moiety activity;
wherein a shift in the spectrum of reporter moiety activity indicates that the protein has undergone a conformation change resulting from the stimulus.

40. The method of claim 39, wherein the stimulus is a mutational alteration of the X protein.

41. The method of claim 39, wherein the stimulus is an alteration in environmental factor.

42. The method of claim 39, wherein the stimulus is a post-translational modification of the X protein.

43. The method of claim 39, wherein the stimulus is contacting the X protein with a test compound in trans.

44. A composition comprising:
1) a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain and X is a nonubiquitin polypeptide; and,
2) a compound that when brought in to contact with, causes conformational change in polypeptide X; and/or,
3) a fusion protein comprising the structure N_{ub}—X′—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy-terminus of the C_{ub} domain and X′ is the X nonubiquitin polypeptide which has been altered by a stimulus.

45. A method of controlling activity of a target gene, comprising:
1) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is a reporter moiety fused to the carboxy-terminus of the C_{ub} domain, wherein the reporter moiety is a gene activating moiety;
2) treating the X polypeptide with a stimulus, thereby causing the cleavage of the RM as a result of a conformational change of the X polypeptide;
wherein the released RM controls activity of the target gene.

46. A method of controlling activity of a protein, comprising:
1) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, X is a nonubiquitin polypeptide, and RM is the protein fused to the carboxy-terminus of the C_{ub} domain, wherein upon cleavage of the C_{ub}—RM junction, the first amino acid of the released RM is an amino acid other than methionine;
2) treating the X polypeptide with a stimulus, thereby causing the cleavage of the RM as a result of a conformational change of the X polypeptide;
wherein the released RM is degraded by N-end rule components, thereby controlling activity of the protein.

47. The method of claim 45 or 46, wherein the stimulus is an alteration in environmental factor, a post-translational modification of the X protein, or contacting the X protein with a test compound in trans.

48. A kit for measuring or detecting protein conformation change caused by a stimulus, comprising:
1) one or more vector constructs for expressing fusion proteins of the general structure N_{ub}—X—C_{ub}—RM, wherein each vector construct comprises a coding sequence for N_{ub}, an amino-terminal ubiquitin domain or a mutant amino-terminal ubiquitin domain selected from Nᵢₐ, N_{ig}, Nᵥᵢ, Nᵢₐ, Nᵥₐ, N_{ig}, N_{vg}, Nₐᵢ, Nₐₐ, N_{ag}, N_{gi}, N_{ga}, or N_{gg}; a coding sequence for C_{ub}, a carboxy-terminal ubiquitin domain; a coding sequences for RM, a reporter moiety fused to the carboxy-terminus of the C_{ub} domain; and at least one cloning site or multicloning site for subcloning the X-protein in-frame with both the N-terminal N_{ub} and the C-terminal C_{ub}—RM moieties; and wherein at least one vector construct expresses a mutant N_{ub} fusion protein;
2) an instruction for using the vector construct of (i) to measure/detect protein conformation change caused by the stimulus.

49. The kit of claim 48, wherein the instruction is not physically associated with the vector constructs of (i).

50. The kit of claim 49, wherein the instruction is posted on a website, or updated periodically, or accessible as a published document.

51. The kit of claim 48, wherein the stimulus is an alteration in environmental factor, a post-translational modification of the X protein, or contacting the X protein with a test compound in trans.

52. A method to detect or measure an alteration of an environmental factor or the presence of a compound in a sample comprising the steps:
1) providing a fusion protein comprising the structure N_{ub}—X—C_{ub}—RM, wherein N_{ub} is an amino-terminal ubiquitin domain or a mutant amino-terminal ubiquitin domain, C_{ub} is a carboxy-terminal ubiquitin domain, RM is a reporter moiety fused to the carboxy terminus of the Cub domain and X is a nonubiquitin polypeptide which changes conformation from said alteration in environmental factor or presence of said compound;
2) contacting the fusion protein with the environment or the sample containing the compound; and,
3) measuring the degree of cleavage of the reporter moiety (RM) from the fusion protein;
wherein a change in the degree of RM activity compared to a standard or control indicates an alternation in said environmental factor or the presence of said compound in the sample.
